SEQUENCE LISTING (108 sequences)

SEQ ID NO: 1 - DNA, 346 bp, Artificial Sequence
  Synthetic
ctcgagtttactccctatcagtgatagagaacgtatgtcgagtttactccctatcagtgatagagaacgatgtcgagtttactccctatcagtgatagagaacgtatgtcgagtttactccctatcagtgatagagaacgtatgtcgagtttactccctatcagtgatagagaacgtatgtcgagtttatccctatcagtgatagagaacgtatgtcgagtttactccctatcagtgatagagaacgtatgtcgaggtaggcgtgtacggtgggaggcctatataagcagagctcgtttagtgaaccgtcagatcgcctggagaattcgagctcggtacccgggga

SEQ ID NO: 2 - DNA, 423 bp, Artificial Sequence
  Synthetic
gtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaagtcgacaccggggcccagatctatcgatcggccggataacgccacc

SEQ ID NO: 3 - DNA, 496 bp, Artificial Sequence
  Synthetic
gaattctccaggcgatctgacggttcactaaacgagctctgcttatataggcctcccaccgtacacgccacctcgacatactcgagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaagtcgacaccggggcccagatctccgcggggatcc

SEQ ID NO: 4 - DNA, 578 bp, Artificial Sequence
  Synthetic
cccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatg

SEQ ID NO: 5 - DNA, 571 bp, Artificial Sequence
  Synthetic
cccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatagttatc

SEQ ID NO: 6 - protein, 397 aa, Unknown
  Adeno-associated virus
MELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 7 - protein, 312 aa, Unknown
  Adeno-associated virus
MELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRLARGHSL

SEQ ID NO: 8 - protein, 621 aa, Unknown
  Adeno-associated virus
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 9 - protein, 536 aa, Unknown
  Adeno-associated virus
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRLARGHSL

SEQ ID NO: 10 - protein, 529 aa, Unknown
  Adeno-associated virus
MASREEEQRETTPERGRGAARRPPTMEDVSSPSPSPPPPRAPPKKRLRRRLESEDEEDSSQDALVPRTPSPRPSTSTADLAIASKKKKKRPSPKPERPPSPEVIVDSEEEREDVALQMVGFSNPPVLIKHGKGGKRTVRRLNEDDPVARGMRTQEEKEESSEAESESTVINPLSLPIVSAWEKGMEAARALMDKYHVDNDLKANFKLLPDQVEALAAVCKTWLNEEHRGLQLTFTSNKTFVTMMGRFLQAYLQSFAEVTYKHHEPTGCALWLHRCAEIEGELKCLHGSIMINKEHVIEMDVTSENGQRALKEQSSKAKIVKNRWGRNVVQISNTDARCCVHDAACPANQFSGKSCGMFFSEGAKAQVAFKQIKAFMQALYPNAQTGHGHLLMPLRCECNSKPGHAPFLGRQLPKLTPFALSNAEDLDADLISDKSVLASVHHPALIVFQCCNPVYRNSRAQGGGPNCDFKISAPDLLNALVMVRSLWSENFTELPRMVVPEFKWSTKHQYRNVSLPVAHSDARQNPFDF

SEQ ID NO: 11 - protein, 294 aa, Unknown
  Adeno-associated virus
MTTSGVPFGMTLRPTRSRLSRRTPYSRDRLPPFETETRATILEDHPLLPECNTLTMHNVSYVRGLPCSVGFTLIQEWVVPWDMVLTREELVILRKCMHVCLCCANIDIMTSMMIHGYESWALHCHCSSPGSLQCIAGGQVLASWFRMVVDGAMFNQRFIWYREVVNYNMPKEVMFMSSVFMRGRHLIYLRLWYDGHVGSVVPAMSFGYSALHCGILNNIVVLCCSYCADLSEIRVRCCARRTRRLMLRAVRIIAEETTAMLYSCRTERRRQQFIRALLQHHRPILMHDYDSTPM

SEQ ID NO: 12 - DNA, 888 bp, Unknown
  Adeno-associated virus
atgactacgtccggcgttccatttggcatgacactacgaccaacacgatctcggttgtctcggcgcactccgtacagtagggatcgcctacctccttttgagacagagacccgcgctaccatactggaggatcatccgctgctgcccgaatgtaacactttgacaatgcacaatgtttcctacgtgcgaggtcttccctgcagtgtgggatttacgctgattcaggaatgggttgttccctgggatatggttctgacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgcatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgtctcatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtagtaa

SEQ ID NO: 13 - DNA, 738 bp, Unknown
  Adeno-associated virus
cgacgtaatccgtagatgtacctggacatccaggtgatgccggcggcggtggtggaggcgcgcggaaagtcgcggacgcggttccagatgttgcgcagcggcaaaaagtgctccatggtcgggacgctctggccggtgaggcgtgcgcagtcgttgacgctctagaccgtgcaaaaggagagcctgtaagcgggcactcttccgtggtctggtggataaattcgcaagggtatcatggcggacgaccggggttcgaaccccggatccggccgtccgccgtgatccatgcggttaccgcccgcgtgtcgaacccaggtgtgcgacgtcagacaacgggggagcgctccttttggcttccttccaggcgcggcggctgctgcgctagcttttttggccactggccgcgcgcggcgtaagcggttaggctggaaagcgaaagcattaagtggctcgctccctgtagccggagggttattttccaagggttgagtcgcaggacccccggttcgagtctcgggccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctccccttctcctaccgcgtcaggaggggcaacatcc

SEQ ID NO: 14 - protein, 735 aa, Unknown [sequence omitted]

SEQ ID NO: 15 - protein, 598 aa, Unknown
  Adeno-associated virus
TAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 16 - protein, 533 aa, Unknown
  Adeno-associated virus
MATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 17 - protein, 204 aa, Unknown
  Adeno-associated virus
LETQTQYLTPSLSDSHQQPPLVWELIRWLQAVAHQWQTITRAPTEWVIPREIGIAIPHGWATESSPPAPEPGPCPPTTTTSTNKFPANQEPRTTITTLATAPLGGILTSTDSTATFHHVTGKDSSTTTGDSDPRDSTSSSLTFKSKRSRRMTVRRRLPITLPARFRCLLTRSTSSRTSSARRIKDASRRSQQTSSWCHSMDTSP

SEQ ID NO: 18 - protein, 19 aa, Unknown
  Adeno-associated virus
ATNFSLLKQAGDVEENPGP

SEQ ID NO: 19 - protein, 18 aa, Artificial Sequence
  Synthetic
EGRGSLLTCGDVEENPGP

SEQ ID NO: 20 - DNA, 1932 bp, Unknown
  Adeno-associated virus
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctga

SEQ ID NO: 21 - protein, 641 aa, Unknown
  Adeno-associated virus
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQMIIRYGCRWLSSRLARGHSL

SEQ ID NO: 22 - protein, 787 aa, Artificial Sequence
  Synthetic
MISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRSDLVPRGSPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 23 - protein, 796 aa, Artificial Sequence
  Synthetic
MISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRAEAAAKEAAAKEAAAKAPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 24 - protein, 702 aa, Artificial Sequence
  Synthetic
MISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRSDLVPRGSPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRLARGHSL

SEQ ID NO: 25 - protein, 711 aa, Artificial Sequence
  Synthetic
MISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRAEAAAKEAAAKEAAAKAPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRLARGHSL

SEQ ID NO: 26 - protein, 690 aa, Artificial Sequence
  Synthetic
MISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGSGASREEEQRETTPERGRGAARRPPTMEDVSSPSPSPPPPRAPPKKRLRRRLESEDEEDSSQDALVPRTPSPRPSTSTADLAIASKKKKKRPSPKPERPPSPEVIVDSEEEREDVALQMVGFSNPPVLIKHGKGGKRTVRRLNEDDPVARGMRTQEEKEESSEAESESTVINPLSLPIVSAWEKGMEAARALMDKYHVDNDLKANFKLLPDQVEALAAVCKTWLNEEHRGLQLTFTSNKTFVTMMGRFLQAYLQSFAEVTYKHHEPTGCALWLHRCAEIEGELKCLHGSIMINKEHVIEMDVTSENGQRALKEQSSKAKIVKNRWGRNVVQISNTDARCCVHDAACPANQFSGKSCGMFFSEGAKAQVAFKQIKAFMQALYPNAQTGHGHLLMPLRCECNSKPGHAPFLGRQLPKLTPFALSNAEDLDADLISDKSVLASVHHPALIVFQCCNPVYRNSRAQGGGPNCDFKISAPDLLNALVMVRSLWSENFTELPRMVVPEFKWSTKHQYRNVSLPVAHSDARQNPFDF

SEQ ID NO: 27 - protein, 455 aa, Artificial Sequence
  Synthetic
MISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGSGTTSGVPFGMTLRPTRSRLSRRTPYSRDRLPPFETETRATILEDHPLLPECNTLTMHNVSYVRGLPCSVGFTLIQEWVVPWDMVLTREELVILRKCMHVCLCCANIDIMTSMMIHGYESWALHCHCSSPGSLQCIAGGQVLASWFRMVVDGAMFNQRFIWYREVVNYNMPKEVMFMSSVFMRGRHLIYLRLWYDGHVGSVVPAMSFGYSALHCGILNNIVVLCCSYCADLSEIRVRCCARRTRRLMLRAVRIIAEETTAMLYSCRTERRRQQFIRALLQHHRPILMHDYDSTPM

SEQ ID NO: 28 - protein, 1000 aa, Artificial Sequence
  Synthetic
MSPKKKRKVEASIEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLRHWVVHNNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFRNKAVHLEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGNSGSGPKKKRKVAAAYPYDVPDYA

SEQ ID NO: 29 - DNA, 23 bp, Artificial Sequence
  Synthetic
cccaggctttgagttgcactcgc

SEQ ID NO: 30 - DNA, 23 bp, Artificial Sequence
  Synthetic
tttgacgtagaattcatgctcca

SEQ ID NO: 31 - DNA, 23 bp, Artificial Sequence
  Synthetic
catccatttcaatcacgtgctcc

SEQ ID NO: 32 - DNA, 23 bp, Artificial Sequence
  Synthetic
aaagtgttacattcgggcagcag

SEQ ID NO: 33 - DNA, 23 bp, Artificial Sequence
  Synthetic
gaagataaccatcggcagccata

SEQ ID NO: 34 - DNA, 23 bp, Artificial Sequence
  Synthetic
gttccatattagtccacgcccac

SEQ ID NO: 35 - DNA, 23 bp, Artificial Sequence
  Synthetic
aattgcacaaagaaaagggcctc

SEQ ID NO: 36 - DNA, 23 bp, Artificial Sequence
  Synthetic
acccagttcacaaagctgtcaga

SEQ ID NO: 37 - DNA, 23 bp, Artificial Sequence
  Synthetic
gagttcaaatttgaacatccggt

SEQ ID NO: 38 - DNA, 23 bp, Artificial Sequence
  Synthetic
agatcaccatcttgtcgacacag

SEQ ID NO: 39 - DNA, 146 bp, Artificial Sequence
  Synthetic
cgacttcttaacccaacagaaggctcgagaaggtatattgctgttgacagtgagcgaaaagtgaaagcacggtgataatagtgaagccacagatgtattatcaccgtgctttcactttctgcctactgcctcggacttcaaggggc

SEQ ID NO: 40 - DNA, 146 bp, Artificial Sequence
  Synthetic
cgacttcttaacccaacagaaggctcgagaaggtatattgctgttgacagtgagcgagctttcatgcaggcgctgtattagtgaagccacagatgtaatacagcgcctgcatgaaagcctgcctactgcctcggacttcaaggggc

SEQ ID NO: 41 - DNA, 146 bp, Artificial Sequence
  Synthetic
cgacttcttaacccaacagaaggctcgagaaggtatattgctgttgacagtgagcgaccccaactgcgacttcaagattagtgaagccacagatgtaatcttgaagtcgcagttggggctgcctactgcctcggacttcaaggggc

SEQ ID NO: 42 - DNA, 146 bp, Artificial Sequence
  Synthetic
cgacttcttaacccaacagaaggctcgagaaggtatattgctgttgacagtgagcgagcatgacactacgaccaacactagtgaagccacagatgtagtgttggtcgtagtgtcatgcctgcctactgcctcggacttcaaggggc

SEQ ID NO: 43 - DNA, 146 bp, Artificial Sequence
  Synthetic
cgacttcttaacccaacagaaggctcgagaaggtatattgctgttgacagtgagcgacgctaccatactggaggatcatagtgaagccacagatgtatgatcctccagtatggtagcgctgcctactgcctcggacttcaaggggc

SEQ ID NO: 44 - DNA, 146 bp, Artificial Sequence
  Synthetic
cgacttcttaacccaacagaaggctcgagaaggtatattgctgttgacagtgagcgacgccatgtttaatcagaggtttagtgaagccacagatgtaaacctctgattaaacatggcgctgcctactgcctcggacttcaaggggc

SEQ ID NO: 45 - DNA, 146 bp, Artificial Sequence
  Synthetic
cgacttcttaacccaacagaaggctcgagaaggtatattgctgttgacagtgagcgaaccaggcctcatacatctccttagtgaagccacagatgtaaggagatgtatgaggcctggtctgcctactgcctcggacttcaaggggc

SEQ ID NO: 46 - DNA, 146 bp, Artificial Sequence
  Synthetic
cgacttcttaacccaacagaaggctcgagaaggtatattgctgttgacagtgagcgaaaagattatgagcctgactaatagtgaagccacagatgtattagtcaggctcataatctttctgcctactgcctcggacttcaaggggc

SEQ ID NO: 47 - DNA, 146 bp, Artificial Sequence
  Synthetic
cgacttcttaacccaacagaaggctcgagaaggtatattgctgttgacagtgagcgattgcaagaccggatgttcaaatagtgaagccacagatgtatttgaacatccggtcttgcaactgcctactgcctcggacttcaaggggc

SEQ ID NO: 48 - DNA, 146 bp, Artificial Sequence
  Synthetic
cgacttcttaacccaacagaaggctcgagaaggtatattgctgttgacagtgagcgagcgcaccaatggcagacaatatagtgaagccacagatgtatattgtctgccattggtgcgcctgcctactgcctcggacttcaaggggc

SEQ ID NO: 49 - DNA, 146 bp, Artificial Sequence
  Synthetic
cgacttcttaacccaacagaaggctcgagaaggtatattgctgttgacagtgagcgagcagtaggacgctcttcattttagtgaagccacagatgtaaaatgaagagcgtcctactgcctgcctactgcctcggacttcaaggggc

SEQ ID NO: 50 - DNA, 146 bp, Artificial Sequence
  Synthetic
cgacttcttaacccaacagaaggctcgagaaggtatattgctgttgacagtgagcgagactttactgtggacactaattagtgaagccacagatgtaattagtgtccacagtaaagtcctgcctactgcctcggacttcaaggggc

SEQ ID NO: 51 - DNA, 12 bp, Artificial Sequence
  Synthetic
attggatccaat

SEQ ID NO: 52 - DNA, 28 bp, Artificial Sequence
  Synthetic
tatttacaaacaaccatgaatgtaagta

SEQ ID NO: 53 - DNA, 18 bp, Artificial Sequence
  Synthetic
cggagtactgtcctccga

SEQ ID NO: 54 - DNA, 30 bp, Artificial Sequence
  Synthetic
atgatacgaaacgtaccgtatcgttaaggt

SEQ ID NO: 55 - DNA, 26 bp, Artificial Sequence
  Synthetic
agaaacaaaccaacctgtctgtatta

SEQ ID NO: 56 - DNA, 28 bp, Artificial Sequence
  Synthetic
aacaaacagacaatctggtctgtttgta

SEQ ID NO: 57 - protein, 248 aa, Artificial Sequence
  Synthetic
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGGPADALDDFDLDMLPADALDDFDLDMLPADALDDFDLDMLPG

SEQ ID NO: 58 - protein, 298 aa, Artificial Sequence
  Synthetic
MDMPRIKPGQRVMMALRKMIASGEIKSGERIAEIPTAAALGVSRMPVRIALRSLEQEGLVVRLGARGYAARGVSSDQIRDAIEVRGVLEGFAARRLAERGMTAETHARFVVLIAEGEALFAAGRLNGEDLDRYAAYNQAFHDTLVSAAGNGAVESALARNGFEPFAAAGALALDLMDLSAEYEHLLAAHRQHQAVLDAVSCGDAEGAERIMRDHALAAIRNAKVFEAAASAGAPLGAAWSIRADSGGGGPTDALDDFDLDMLPADALDDFDLDMLPADALDDFDLDMLPGPPKKKRKV

SEQ ID NO: 59 - protein, 264 aa, Artificial Sequence
  Synthetic
MVRRTKEEAQETRAQIIEAAERAFYKRGVARTTLADIAELAGVTRGAIYWHFNNKAELVQALLDSLHETHDHLARASESEDEVDPLGCMRKLLLQVFNELVLDARTRRINEILHHKCEFTDDMCEIRQQHQSAVLDCHKGITLTLANVVRRGQLPGELDAERAAVAMFAYVDGLIRRWLLLPDSVDLLGDVEKWVDTGLDMLRLSPALRKSGGGGPTDALDDFDLDMLPADALDDFDLDMLPADALDDFDLDMLPGPPKKKRKV

SEQ ID NO: 60 - protein, 254 aa, Artificial Sequence
  Synthetic
MARTPSRSSIGSLRSPHTHKAILTSTIEILKECGYSGLSIESVARRAGAGKPTIYRWWTNKAALIAEVYENEIEQVRKFPDLGSFKADLDFLLHNLWKVWRETICGEAFRCVIAEAQLDPVTLTQLKDQFMERRREIPKKLVEDAISNGELPKDINRELLLDMIFGFCWYRLLTEQLTVEQDIEEFTFLLINGVCPGTQCSGGGGPTDALDDFDLDMLPADALDDFDLDMLPADALDDFDLDMLPGPPKKKRKV

SEQ ID NO: 61 - protein, 257 aa, Artificial Sequence
  Synthetic
MSPKRRTQAERAMETQGKLIAAALGVLREKGYAGFRIADVPGAAGVSRGAQSHHFPTKLELLLATFEWLYEQITERSRARLAKLKPEDDVIQQMLDDAAEFFLDDDFSIGLDLIVAADRDPALREGIQRTVERNRFVVEDMWLGVLVSRGLSRDDAEDILWLIFNSVRGLVVRSLWQKDKERFERVRNSTLEIARERYAKFKRSGGGGPTDALDDFDLDMLPADALDDFDLDMLPADALDDFDLDMLPGPPKKKRKV

SEQ ID NO: 62 - protein, 40 aa, Artificial Sequence
  Synthetic
NPDFPPEVEEQDASTLPVSCAWESGMKRHRAACASASINV

SEQ ID NO: 63 - protein, 40 aa, Artificial Sequence
  Synthetic
NPDFPPEVEEQAASTLPVSCAWESGMKRHRAACASASINV

SEQ ID NO: 64 - protein, 40 aa, Artificial Sequence
  Synthetic
NPDFPPEVEEQDASALPVSCAWESGMKRHRAACASASINV

SEQ ID NO: 65 - protein, 40 aa, Artificial Sequence
  Synthetic
NPDFPPEVEEQDASTLPVSAAWESGMKRHRAACASASINV

SEQ ID NO: 66 - protein, 40 aa, Artificial Sequence
  Synthetic
NPDFPPEVEEQDASTLPVSCAWEAGMKRHRAACASASINV

SEQ ID NO: 67 - DNA, 48 bp, Artificial Sequence
  Synthetic
  misc_feature (22)..(26): n is a, c, g, or t
aaagtgaaagcacggtgataannnnnttatcaccgtgctttcactttc

SEQ ID NO: 68 - DNA, 48 bp, Artificial Sequence
  Synthetic
  misc_feature (22)..(26): n is a, c, g, or t
gctttcatgcaggcgctgtatnnnnnatacagcgcctgcatgaaagcc

SEQ ID NO: 69 - DNA, 48 bp, Artificial Sequence
  Synthetic
  misc_feature (22)..(26): n is a, c, g, or t
ccccaactgcgacttcaagatnnnnnatcttgaagtcgcagttggggc

SEQ ID NO: 70 - DNA, 48 bp, Artificial Sequence
  Synthetic
  misc_feature (22)..(26): n is a, c, g, or t
gcatgacactacgaccaacacnnnnngtgttggtcgtagtgtcatgcc

SEQ ID NO: 71 - DNA, 48 bp, Artificial Sequence
  Synthetic
  misc_feature (22)..(26): n is a, c, g, or t
cgctaccatactggaggatcannnnntgatcctccagtatggtagcgc

SEQ ID NO: 72 - DNA, 48 bp, Artificial Sequence
  Synthetic
  misc_feature (22)..(26): n is a, c, g, or t
cgccatgtttaatcagaggttnnnnnaacctctgattaaacatggcgc

SEQ ID NO: 73 - DNA, 48 bp, Artificial Sequence
  Synthetic
  misc_feature (22)..(26): n is a, c, g, or t
accaggcctcatacatctcctnnnnnaggagatgtatgaggcctggtc

SEQ ID NO: 74 - DNA, 48 bp, Artificial Sequence
  Synthetic
  misc_feature (22)..(26): n is a, c, g, or t
aaagattatgagcctgactaannnnnttagtcaggctcataatctttc

SEQ ID NO: 75 - DNA, 48 bp, Artificial Sequence
  Synthetic
  misc_feature (22)..(26): n is a, c, g, or t
ttgcaagaccggatgttcaaannnnntttgaacatccggtcttgcaac

SEQ ID NO: 76 - DNA, 48 bp, Artificial Sequence
  Synthetic
  misc_feature (22)..(26): n is a, c, g, or t
gcgcaccaatggcagacaatannnnntattgtctgccattggtgcgcc

SEQ ID NO: 77 - DNA, 48 bp, Artificial Sequence
  Synthetic
  misc_feature (22)..(26): n is a, c, g, or t
gcagtaggacgctcttcatttnnnnnaaatgaagagcgtcctactgcc

SEQ ID NO: 78 - DNA, 48 bp, Artificial Sequence
  Synthetic
  misc_feature (22)..(26): n is a, c, g, or t
gactttactgtggacactaatnnnnnattagtgtccacagtaaagtcc

SEQ ID NO: 79 - protein, 260 aa, Artificial Sequence
  Synthetic
MVIMSPKRRTQAERAMETQGKLIAAALGVLREKGYAGFRIADVPGAAGVSRGAQSHHFPTKLELLLATFEWLYEQITERSRARLAKLKPEDDVIQQMLDDAAEFFLDDDFSIGLDLIVAADRDPVLREGIQRTVERNRFVVGDIWLGVLVSRGLSRDDAEDILWLIFNSVRGLVVRSLWQKDKERFERVRNSTLEIARERYAKFKRSGGGGPTDALDDFDLDMLPADALDDFDLDMLPADALDDFDLDMLPGPPKKKRKV

SEQ ID NO: 80 - protein, 119 aa, Unknown
  Adeno-associated virus
LAHHHQSPQSGIRTTAGVLCFLGTSTSDPSTDSTRESRSTRQTPRPSSTTKPTTGSSTAETTRTSSTTTPTRSFRSALKKIRLLGATSDEQSSRRKRGFLNLWAWLRNLLRRLREKRGR

SEQ ID NO: 81 - protein, 159 aa, Artificial Sequence
  Synthetic
MISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERR

SEQ ID NO: 82 - protein, 559 aa, Artificial Sequence
  Synthetic
MISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGSGMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 83 - protein, 474 aa, Artificial Sequence
  Synthetic
MISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGSGMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRLARGHSL

SEQ ID NO: 84 - protein, 366 aa, Artificial Sequence
  Synthetic
MISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGSGMETQTQYLTPSLSDSHQQPPLVWELIRWLQAVAHQWQTITRAPTEWVIPREIGIAIPHGWATESSPPAPEPGPCPPTTTTSTNKFPANQEPRTTITTLATAPLGGILTSTDSTATFHHVTGKDSSTTTGDSDPRDSTSSSLTFKSKRSRRMTVRRRLPITLPARFRCLLTRSTSSRTSSARRIKDASRRSQQTSSWCHSMDTSP

SEQ ID NO: 85 - protein, 1601 aa, Artificial Sequence
  Synthetic
MTTTMKISIEFLEPFRMTKWQESTRRNKNNKEFVRGQAFARWHRNKKDNTKGRPYITGTLLRSAVIRSAENLLTLSDGKISEKTCCPGKFDTEDKDRLLQLRQRSTLRWTDKNPCPDNAETYCPFCELLGRSGNDGKKAEKKDWRFRIHFGNLSLPGKPDFDGPKAIGSQRVLNRVDFKSGKAHDFFKAYEVDHTRFPRFEGEITIDNKVSAEARKLLCDSLKFTDRLCGALCVIRFDEYTPAADSGKQTENVQAEPNANLAEKTAEQIISILDDNKKTEYTRLLADAIRSLRRSSKLVAGLPKDHDGKDDHYLWDIGKKKKDENSVTIRQILTTSADTKELKNAGKWREFCEKLGEALYLKSKDMSGGLKITRRILGDAEFHGKPDRLEKSRSVSIGSVLKETVVCGELVAKTPFFFGAIDEDAKQTDLQVLLTPDNKYRLPRSAVRGILRRDLQTYFDSPCNAELGGRPCMCKTCRIMRGITVMDARSEYNAPPEIRHRTRINPFTGTVAEGALFNMEVAPEGIVFPFQLRYRGSEDGLPDALKTVLKWWAEGQAFMSGAASTGKGRFRMENAKYETLDLSDENQRNDYLKNWGWRDEKGLEELKKRLNSGLPEPGNYRDPKWHEINVSIEMASPFINGDPIRAAVDKRGTDVVTFVKYKAEGEEAKPVCAYKAESFRGVIRSAVARIHMEDGVPLTELTHSDCECLLCQIFGSEYEAGKIRFEDLVFESDPEPVTFDHVAIDRFTGGAADKKKFDDSPLPGSPARPLMLKGSFWIRRDVLEDEEYCKALGKALADVNNGLYPLGGKSAIGYGQVKSLGIKGDDKRISRLMNPAFDETDVAVPEKPKTDAEVRIEAEKVYYPHYFVEPHKKVEREEKPCGHQKFHEGRLTGKIRCKLITKTPLIVPDTSNDDFFRPADKEARKEKDEYHKSYAFFRLHKQIMIPGSELRGMVSSVYETVTNSCFRIFDETKRLSWRMDADHQNVLQDFLPGRVTADGKHIQKFSETARVPFYDKTQKHFDILDEQEIAGEKPVRMWVKRFIKRLSLVDPAKHPQKKQDNKWKRRKEGIATFIEQKNGSYYFNVVTNNGCTSFHLWHKPDNFDQEKLEGIQNGEKLDCWVRDSRYQKAFQEIPENDPDGWECKEGYLHVVGPSKVEFSDKKGDVINNFQGTLPSVPNDWKTIRTNDFKNRKRKNEPVFCCEDDKGNYYTMAKYCETFFFDLKENEEYEIPEKARIKYKELLRVYNNNPQAVPESVFQSRVARENVEKLKSGDLVYFKHNEKYVEDIVPVRISRTVDDRMIGKRMSADLRPCHGDWVEDGDLSALNAYPEKRLLLRHPKGLCPACRLFGTGSYKGRVRFGFASLENDPEWLIPGKNPGDPFHGGPVMLSLLERPRPTWSIPGSDNKFKVPGRKFYVHHHAWKTIKDGNHPTTGKAIEQSPNNRTVEALAGGNSFSFEIAFENLKEWELGLLIHSLQLEKGLAHKLGMAKSMGFGSVEIDVESVRLRKDWKQWRNGNSEIPNWLGKGFAKLKEWFRDELDFIENLKKLLWFPEGDQAPRVCYPMLRKKDDPNGNSGYEELKDGEFKKEDRQKKLTTPWTPWA

SEQ ID NO: 86 - protein, 4 aa, Artificial Sequence
  Synthetic
GGGS

SEQ ID NO: 87 - protein, 6 aa, Artificial Sequence
  Synthetic
NAAAEF

SEQ ID NO: 88 - protein, 8 aa, Artificial Sequence
  Synthetic
GATRLPGS

SEQ ID NO: 89 - protein, 5 aa, Artificial Sequence
  Synthetic
EAAAK

SEQ ID NO: 90 - protein, 15 aa, Artificial Sequence
  Synthetic
GGGGSGGGGSGGGGS

SEQ ID NO: 91 - protein, 8 aa, Artificial Sequence
  Synthetic
GGGGGGGG

SEQ ID NO: 92 - protein, 6 aa, Artificial Sequence
  Synthetic
GGGGGG

SEQ ID NO: 93 - protein, 5 aa, Artificial Sequence
  Synthetic
  REPEAT (1)..(5): may repeat 1-3 times
EAAAK

SEQ ID NO: 94 - protein, 46 aa, Artificial Sequence
  Synthetic
AEAAAKEAAAKEAAAKEAAAKALEAEAAAKEAAAKEAAAKEAAAKA

SEQ ID NO: 95 - protein, 5 aa, Artificial Sequence
  Synthetic
GGGGS

SEQ ID NO: 96 - protein, 5 aa, Artificial Sequence
  Synthetic
PAPAP

SEQ ID NO: 97 - protein, 12 aa, Artificial Sequence
  Synthetic
AEAAAKEAAAKA

SEQ ID NO: 98 - protein, 5 aa, Artificial Sequence
  Synthetic
  REPEAT (1)..(5): May repeat 1, 2, or 4 times
GGGGS

SEQ ID NO: 99 - protein, 2 aa, Artificial Sequence
  Synthetic
  REPEAT (1)..(2): May repeat 5-17 times
AP

SEQ ID NO: 100 - protein, 17 aa, Artificial Sequence
  Synthetic
VSQTSKLTRAETVFPDV

SEQ ID NO: 101 - protein, 6 aa, Artificial Sequence
  Synthetic
PLGLWA

SEQ ID NO: 102 - protein, 6 aa, Artificial Sequence
  Synthetic
RVLAEA

SEQ ID NO: 103 - protein, 10 aa, Artificial Sequence
  Synthetic
EDVVCCSMSY

SEQ ID NO: 104 - protein, 9 aa, Artificial Sequence
  Synthetic
GGIEGRGSC

SEQ ID NO: 105 - protein, 10 aa, Artificial Sequence
  Synthetic
TRHRQPRGWE

SEQ ID NO: 106 - protein, 10 aa, Artificial Sequence
  Synthetic
AGNRVRRSVG

SEQ ID NO: 107 - protein, 9 aa, Artificial Sequence
  Synthetic
RRRRRRRRR

SEQ ID NO: 108 - protein, 4 aa, Artificial Sequence
  Synthetic
GFLG

(19) United States
(12) Patent Application Publication
(10) Pub. No.: US 2024/0247283 A1
(43) Pub. Date: Jul. 25, 2024

(54) COMPOSITIONS AND METHODS FOR ADENO-ASSOCIATED VIRAL PRODUCTION

(71) Applicant: Asimov Inc., Boston, MA (US)

(72) Inventors: Jeremy J. Gam, Boston, MA (US); Brianna E.K. Jayanthi, Boston, MA (US); Michael T. Leonard, Boston, MA (US); Alec A.K. Nielsen, Boston, MA (US)

(73) Assignee: Asimov Inc., Boston, MA (US)

(21) Appl. No.: 18/289,234

(22) PCT Filed: May 6, 2022
(86) PCT No.: PCT/US2022/028111

§ 371 Date: Nov. 2, 2023

Related U.S. Application Data
(60) Provisional application No. 63/185,066, filed on May 6, 2021.

(51) Int. Cl. C12N 15/86; C07K 14/005; C12N 5/071; C12N 9/22

(52) U.S. Cl. CPC C12N 15/86; C07K 14/005; C12N 5/0686; C12N 9/22; C12N 2310/20; C12N 2750/14122; C12N 2750/14143; C12N 2750/14152

(57) ABSTRACT

Disclosed herein are cell genetically engineered cell for AAV production. The genetically engineered cell comprises molecular systems for temporal control of expression of genes required for AAV production. Also disclosed herein are methods of using genetically engineered cells for AAV production.

### RELATED APPLICATIONS

This application claims the benefit under 35 U.S.C. § 119 of U.S. provisional application Ser. No. 63/185,066, filed May 6, 2021, the entire contents of which are incorporated by reference herein.

### FIELD

Described herein are Adeno-Associated Virus (AAV) production systems. Also described herein are engineered cells and kits comprising an AAV production system and methods of using the same for AAV production.

### REFERENCE TO A SEQUENCE LISTING SUBMITTED AS A TEXT FILE VIA EFS-WEB

This application contains a Sequence Listing which has been submitted in ASCII format via EFS-Web and is hereby incorporated by reference in its entirety. Said ASCII copy, created on May 3, 2022, is named A121070008WO00-SEQ-ARM and is 161,100 bytes in size.

### BACKGROUND OF INVENTION

AAV are a promising gene delivery modality for cell and gene therapy. AAV can be modified to carry therapeutic genetic payloads to cells within a subject. The production of AAV normally entails transient transfection of plasmids containing genes required for viral vector production into cell culture. However, transient transfection has several shortfalls. Large quantities of DNA and transfection reagent must be procured for the transfection process, which is costly. Also, poor transfection efficiency can result in minimal numbers of ‘transfected’ cells and increased variation associated with transfection steps and viral production.

### SUMMARY OF INVENTION

Described herein are AAV production systems that introduce inducible control of gene products required for AAV production including cytostatic or cytotoxic gene products. This inducible control can be mediated at the transcriptional level (i.e., inducible control of mRNA concentration) or at the post-translational level (inducible control of protein expression). Each of the described AAV production systems can be integrated into the genome using random integration, targeted integration, or transposon-mediated integration.

In some aspects, this application describes an engineered cell for AAV production, comprising one or more stably integrated nucleic acid molecules collectively comprising nucleic acid sequences encoding for: Rep52 or Rep40; DD-Rep78 or DD-Rep68; DD-E2A; DD-E4Orf6; VARNA; VP1; VP2; VP3; AAP; each of which is operably linked to a promoter; wherein:
(i) DD-E2A comprises the amino acid sequence of E2A linked directly or indirectly to a molecule binding degradation domain;
(ii) DD-E4orf6 comprises the amino acids sequence of E4orf6 linked directly or indirectly to a molecule binding degradation domain;
(iii) DD-Rep78 comprises the amino acid sequence of Rep78 linked directly or indirectly to a molecule binding degradation domain; and
(iv) DD-Rep68 comprises the amino acid sequence of Rep68 linked directly or indirectly to a molecule binding degradation domain.

In some embodiments, the degradation domain is selected from the group consisting of FKBP or ecDHFR degradation domains. In some embodiments, the ecDHFR degradation domain is stabilized by the small molecules Trimethoprim. In some embodiments, the FKBP degradation domain is stabilized by the small molecules Shield, Shield2 or SLF*.

In some embodiments, the one or more stably integrated nucleic acid molecules comprises a first stably integrated nucleic acid molecule comprising the nucleic acid sequence encoding for Rep52 or Rep40, the nucleic acid sequence encoding for DD-Rep78 or DD-Rep68, the nucleic acid sequence encoding for VP1, the nucleic acid sequence encoding for VP2, the nucleic acid sequence encoding for VP3, and the nucleic acid sequence encoding for AAP. In some embodiments, the first stably integrated nucleic acid molecule further comprises a selection marker that is operably linked to a promoter.

In some embodiments, the one or more stably integrated nucleic acid molecules comprises a second stably integrated nucleic acid molecule comprising the nucleic acid sequence encoding for DD-E2A, the nucleic acid sequence encoding for DD-E4orf6, and the nucleic acid sequence encoding for VARNA.

In some embodiments, the second stably integrated nucleic acid molecule further comprises a selection marker that is operably linked to a promoter. In some embodiments, the engineered cell comprises a nucleic acid sequence encoding for Rep52, wherein the nucleic acid sequence encoding for Rep52 comprises the amino acid sequence of SEQ ID NO: 6. In some embodiments, the engineered cell comprises a nucleic acid sequence encoding for Rep40, wherein the nucleic acid sequence encoding for Rep40 comprises the amino acid sequence of SEQ ID NO: 7. In some embodiments, the engineered cell comprises a nucleic acid sequence encoding for DD-Rep78, wherein the nucleic acid sequence encoding for DD-Rep78 the amino acid sequence of SEQ ID NO: 22 or SEQ ID NO: 23.

In some embodiments, the engineered cell comprises a nucleic acid sequence encoding for DD-Rep68, wherein the nucleic acid sequence encoding for DD-Rep68 comprises the amino acid sequence of SEQ ID NO: 24 or SEQ ID NO: 25. In some embodiments, the nucleic acid sequence encoding for VP1 comprises the amino acid sequence of SEQ ID NO: 14. In some embodiments, the nucleic acid sequence encoding for VP2 comprises the amino acid sequence of SEQ ID NO: 15. In some embodiments, the nucleic acid sequence encoding for VP3 comprises the amino acid sequence of SEQ ID NO: 16. In some embodiments, the nucleic acid sequence encoding for AAP comprises the amino acid sequence of SEQ ID NO: 17. In some embodiments, the nucleic acid sequence encoding for VARNA comprises the nucleic acid sequence of SEQ ID NO: 13. In some embodiments, the nucleic acid sequence encoding for DD-E2A comprises the amino acid sequence of SEQ ID NO: 26. In some embodiments, the nucleic acid sequence encoding for DD-E4orf6 comprises the amino acid sequence of SEQ ID NO: 27.

In some aspects, this application discloses a kit comprising an engineered cell described herein. In some embodiments, the kit further comprising a nucleic acid molecule comprising, from 5′ to 3′: (i) a nucleic acid sequence of a 5′ inverted tandem repeat; (ii) a multiple cloning site; and (iii) a nucleic acid sequence of a 3′ inverted tandem repeat. In some embodiments, the nucleic acid molecule is a plasmid or a vector.

In some aspects, this application discloses a method for AAV production, comprising culturing an engineered cell described herein with a molecule capable of binding the molecule binding degradation domain. In some embodiments, the molecule is Trimethoprim or Shield. In some embodiments, culturing the engineered cell with the molecule increases the concentration of proteins comprising the degradation domain.

In some aspects, this application discloses an engineered cell for AAV production, comprising one or more stably integrated nucleic acid molecules collectively comprising a nucleic acid sequence encoding for each of: Rep52 or Rep40; Rep78 or Rep68; E2A; E4Orf6; VARNA; VP1; VP2; VP3; AAP; one or more crRNAs; and Cas13 or Cas7-11, wherein the nucleic acid sequences of one or more of Rep52 or Rep40, Rep78 or Rep68, E2A, and E4Orf6 further comprises a nucleic acid sequence that complements at least one of the one or more crRNAs.

In some embodiments, the one or more stably integrated nucleic acid molecules comprises a first stably integrated nucleic acid molecule comprising the nucleic acid sequence encoding Cas13. In some embodiments, the nucleic acid sequence encoding Cas13 encodes RfxCas13d. In some embodiments, the nucleic acid sequence encoding RfxCas13d comprises SEQ ID NO: 28. In some embodiments, the nucleic acid sequence encoding Cas13 further comprises a degradation domain.

In some embodiments, the degradation domain is the c-terminal domain of ornithine decarboxylase (ODC) degradation domain, the M-ODC degradation domain containing an alanine substitution at amino acid position 12, 15, 20 or 24 (SEQ ID NOs: 62-66) or the auxin-inducible degron. In some embodiments, the degradation domain is downstream of Cas13.

In some embodiments, the nucleic acid sequence encoding Cas13 is operably linked to a first chemically inducible promoter. In some embodiments, the first chemically inducible promoter is selected from the group consisting of pTRE3G, pTREtight, and a promoters containing at least one of VanR, TtgR, PhIF, or CymR, or the Gal4 UAS operator sequences In some embodiments, the nucleic acid sequence encoding the first chemically inducible promoter is any one of SEQ ID NO: 1-3 or comprises any one of SEQ ID NOs: 51-56.

In some embodiments, the one or more stably integrated nucleic acid molecules comprises a first stably integrated nucleic acid molecule comprising the nucleic acid sequence encoding Cas7-11. In some embodiments, the nucleic acid sequence encoding Cas7-11 encodes DiCas7-11. In some embodiments, the nucleic acid sequence encoding DiCas7-11 comprises SEQ ID NO: 85. In some embodiments, the nucleic acid sequence encoding Cas7-11 further comprises a degradation domain. In some embodiments, the degradation domain is the c-terminal domain of ornithine decarboxylase (ODC) degradation domain, the M-ODC degradation domain containing an alanine substitution at amino acid position 12, 15, 20 or 24 (SEQ ID NOs: 62-66), or the auxin-inducible degron. In some embodiments, the degradation domain is downstream of Cas7-11.

In some embodiments, the nucleic acid sequence encoding Cas7-11 is operably linked to a first chemically inducible promoter. In some embodiments, the first chemically inducible promoter is selected from the group consisting of pTRE3G, pTREtight, and a promoters containing at least one of VanR, TtgR, PhIF, or CymR, or the Gal4 UAS operator sequences. In some embodiments, the nucleic acid sequence encoding the first chemically inducible promoter is any one of SEQ ID NO: 1-3 or comprises any one of SEQ ID NOs: 51-56. In some embodiments, the first stably integrated nucleic acid molecule further comprises a selection marker that is operably linked to a promoter.

In some embodiments, the one or more stably integrated nucleic acid molecules comprises a second stably integrated nucleic acid molecule comprising the nucleic acid sequence encoding one or more crRNAs. In some embodiments, the one or more crRNAs each comprise a nucleic acid sequence that is complementary to any one of the nucleic acid sequences encoding for Rep52 or Rep40, Rep78 or Rep68, E2A, and E4Orf6.

In some embodiments, the one or more crRNAs each comprise a nucleic acid sequence of any one of SEQ ID NOs: 29-38. In some embodiments, the nucleic acid sequence encoding for the one or more crRNAs is operably linked to a second chemically inducible promoter. In some embodiments, the second chemically inducible promoter is selected from the group consisting of pTRE3G, pTREtight, or a promoter containing at least one of VanR, TtgR, PhIF, or CymR, or the Gal4 UAS operator sequences. In some embodiments, the nucleic acid sequence encoding the second chemically inducible promoter is any one of SEQ ID NOs: 1-2 or comprises any one of SEQ ID NOs: 51-56. In some embodiments, the second stably integrated nucleic acid molecule further comprises a selection marker that is operably linked to a promoter.

In some embodiments, the one or more stably integrated nucleic acid molecules comprises a third stably integrated nucleic acid molecule wherein the third stably integrated nucleic acid molecule comprises the nucleic acid sequence encoding for Rep52 or Rep40. In some embodiments, the nucleic acid sequence encoding for Rep52 comprises an amino acid sequence of SEQ ID NO: 6. In some embodiments, the nucleic acid sequence encoding for Rep40 comprises an amino acid sequence of SEQ ID NO: 7. In some embodiments, the third stably integrated nucleic acid molecule comprises a nucleic acid sequence encoding for Rep78 or Rep68. In some embodiments, the nucleic acid sequence encoding for Rep78 comprises an amino acid sequence of SEQ ID NO: 8. In some embodiments, the nucleic acid sequence encoding for Rep68 comprises an amino acid sequence of SEQ ID NO: 9.

In some embodiments, the third stably integrated nucleic acid molecule comprises nucleic acid sequences encoding for VP1, VP2, and VP3. In some embodiments, the nucleic acid sequence encoding for VP1 comprises the amino acid sequence of SEQ ID NO: 14. In some embodiments, the nucleic acid sequence encoding for VP2 comprises the amino acid sequence of SEQ ID NO: 15. In some embodiments, the nucleic acid sequence encoding for VP3 comprises the amino acid sequence of SEQ ID NO: 16. In some embodiments, the third stably integrated nucleic acid molecule comprises a nucleic acid sequence encoding for AAP. In some embodiments, the nucleic acid sequence encoding for AAP comprises the amino acid sequence of SEQ ID NO: 17.

In some embodiments, the one or more stably integrated nucleic acid molecules comprises a fourth stably integrated nucleic acid molecule comprising the nucleic acid sequences encoding for E2A, E4Orf6, and VARNA. In some embodiments, the nucleic acid sequence encoding for E2A comprises the amino acid sequence of SEQ ID NO: 10. In some embodiments, the nucleic acid sequence encoding for E4Orf6 comprises the amino acid sequence of SEQ ID NO: 11. In some embodiments, the nucleic acid sequence encoding for VARNA comprises the nucleic acid sequence of SEQ ID NO: 13.

In some embodiments, the fourth stably integrated nucleic acid molecule further comprises a selection marker that is operably linked to a promoter. In some embodiments, the nucleic acid sequence encoding for Cas13 or Cas7-11 and/or the nucleic acid sequence encoding for one or more crRNAs is operably linked to a chemically inducible promoter, and wherein the engineered cell further comprises a fifth stably integrated nucleic acid molecule, wherein the fifth stably integrated nucleic acid molecule comprises a nucleic acid sequence encoding for a transcriptional activator that, when expressed in the presence of a small molecule inducer, binds to the chemically inducible promoter of the engineered cell. In some embodiments, the transcriptional activator is selected from the group consisting of TetOff, TetOff-Advanced, VanR-VP16, TtgR-VP16, PhIF-VP16, and the cumate-responsive transactivators cTA and rcTA.

In some embodiments, the small molecule inducer is selected from the group consisting of lactose, arabinose, and doxycycline. In some embodiments, wherein the transcriptional activator is TetOff and the small molecule inducer is doxycycline. In some embodiments, wherein the engineered cell is HEK293 cell or HeLa cell.

In some aspects, the present disclosure describes a kit comprising an engineered cell described herein. In some embodiments, the kit further comprising a nucleic acid molecule comprising, from 5′ to 3′: (i) a nucleic acid sequence of a 5′ inverted tandem repeat; (ii) a multiple cloning site; and (iii) a nucleic acid sequence of a 3′ inverted tandem repeat. In some embodiments, the nucleic acid molecule is a plasmid or a vector.

In some aspects this disclosure describes a method for AAV production, comprising culturing an engineered cell described herein in the presence of a molecule that binds to the degradation domain and increases degradation of the degradation domain, thereby inducing AAV production, optionally wherein the molecule is Auxin or antizyme.

In some aspects this disclosure describes a method for AAV production, comprising culturing an engineered cell described herein in the presence of a molecule that binds to the degradation domain and decreases degradation of the degradation domain and then culturing the engineered cell in the absence of the molecule, thereby inducing AAV production, wherein the degradation domain is selected from the group consisting of FKBP or ecDHFR and optionally wherein the molecule is trimethoprim, Shield, Shield2 or SLF*.

In some aspects this disclosure describes a method for AAV production, comprising culturing an engineered cell described herein in the presence of the small molecule inducer corresponding to the transcriptional activator that binds to the chemically inducible promoter, thereby inducing AAV production. In some embodiments, the small molecule inducer is selected from the group consisting of lactose, arabinose, and doxycycline.

In some aspects, this application discloses an engineered cell for AAV production, comprises one or more nucleic acid molecules collectively comprising a nucleic acid sequence encoding for each of: Rep78 or Rep68; Rep52 or Rep40; VP1; VP2; VP3; E2A; E4orf6; VARNA; and shRNAs each of which is operably linked to a promoter, wherein one or more shRNAs comprise a sequence that is complementary to the mRNA of one or more of Rep78 or Rep68; Rep52 or Rep40; VP1; VP2; VP3; AAP; E2A; E4orf6; and VARNA.

In some embodiments, the one or more nucleic acid molecules comprises a first nucleic acid molecule comprising the nucleic acid sequence encoding for one or more shRNAs. In some embodiments, the one or more shRNAs each comprise a nucleic acid sequence that is complementary to any one of the nucleic acid sequences encoding for Rep78 or Rep68; Rep52 or Rep40; VP1; VP2; VP3; AAP; E2A; E4orf6; and VARNA. In some embodiments, the one or more shRNAs comprise a nucleic acid sequence of any one of SEQ ID NO: 39-50.

In some embodiments, the nucleic acid sequence encoding for the one or more shRNAs is operably linked to a chemically inducible promoter. In some embodiments, the chemically inducible promoter is selected from the group consisting of pTRE3G, pTREtight, or a promoter containing at least one of VanR, TtgR, PhIF, or CymR, or the Gal4 UAS operator sequences. In some embodiments, the nucleic acid sequence encoding the chemically inducible promoter is any one of SEQ ID NOs: 1-2 or comprises any one of SEQ ID NOs: 51-56.

In some embodiments, the first nucleic acid molecule further comprises a nucleic acid sequence encoding for a selection marker that is operably linked to a promoter. In some embodiments, the first nucleic acid molecule further comprises a nucleic acid sequence encoding for a Neo-TagBFP.

In some embodiments, the first nucleic acid molecule is a plasmid or a vector. In some embodiments, the first nucleic acid molecule is stably integrated.

In some embodiments, the one or more stably integrated nucleic acid molecules comprises a second stably integrated nucleic acid molecule comprising the nucleic acid sequences encoding for Rep78 or Rep68; Rep52 or Rep40; VP1; VP2; VP3; and AAP. In some embodiments, the second stably integrated nucleic acid molecule comprises the nucleic acid sequence encoding for Rep52 or Rep40. In some embodiments, the nucleic acid sequence encoding for Rep52 comprises an amino acid sequence of SEQ ID NO: 6. In some embodiments, the nucleic acid sequence encoding for Rep40 comprises an amino acid sequence of SEQ ID NO: 7. In some embodiments, the second stably integrated nucleic acid molecule comprises an amino acid sequence encoding for Rep78 or Rep68. In some embodiments, the nucleic acid sequence encoding for Rep78 comprises an amino acid sequence of SEQ ID NO: 8. In some embodiments, the nucleic acid sequence encoding for Rep68 comprises an amino acid sequence of SEQ ID NO: 9.

In some embodiments, the second stably integrated nucleic acid molecule comprises an amino acid sequence encoding for VP1, VP2, or VP3. In some embodiments, the nucleic acid sequence encoding for VP1 comprises an amino acid sequence of SEQ ID NO: 14. In some embodiments, the nucleic acid sequence encoding for VP2 comprises an amino acid sequence of SEQ ID NO: 15. In some embodiments, the nucleic acid sequence encoding for VP3 comprises an amino acid sequence of SEQ ID NO: 16. In some embodiments, the second stably integrated nucleic acid molecule comprises a nucleic acid sequence encoding for AAP. In some embodiments, the nucleic acid sequence encoding for AAP comprises the amino acid sequence of SEQ ID NO: 17.

In some embodiments, the one or more stably integrated nucleic acid molecules comprises a third stably integrated nucleic acid molecule comprising the nucleic acid sequences encoding for E2A, E4Orf6, and VARNA. In some embodiments, the nucleic acid sequence encoding for E2A comprises the amino acid sequence of SEQ ID NO: 10. In some embodiments, the nucleic acid sequence encoding for E4Orf6 comprises the amino acid sequence of SEQ ID NO: 11. In some embodiments, the nucleic acid sequence encoding for VARNA comprises the amino acid sequence of SEQ ID NO: 13. In some embodiments, the third stably integrated nucleic acid molecule further comprises a selection marker that is operably linked to a promoter.

In some embodiments, the engineered cell further comprises a fourth stably integrated nucleic acid molecule, wherein the fourth stably integrated nucleic acid molecule comprises a nucleic acid sequence encoding for a transcriptional activator that, when expressed in the presence of a small molecule inducer, binds to a chemically inducible promoter of the engineered cell, and the nucleic acid sequences encoding the one or more crRNAs. In some embodiments, the transcriptional activator is selected from the group consisting of Tet-On 3G, TetOff-Advanced, VanR-VP16, TtgR-VP16, PhIF-VP16, and the cumate-responsive transactivators cTA and rcTA. In some embodiments, the small molecule inducer is selected from the group consisting of lactose, arabinose, and doxycycline. In some embodiments, the transcriptional activator is TET-On 3G and the small molecule inducer is doxycycline. In some embodiments, the engineered cell is HEK293 cell or HeLa cell.

In some aspects, this application discloses a kit comprising an engineered cell as described above. In some embodiments, the kit further comprises a nucleic acid sequence comprising, from 5′ to 3′: (i) a nucleic acid sequence of a 5′ inverted tandem repeat; (ii) a multiple cloning site; and (iii) a nucleic acid sequence of a 3′ inverted tandem repeat. In some embodiments, the nucleic acid sequence is a plasmid or a vector.

In some aspects, this application discloses a method for AAV production, comprising culturing an engineered cell as described above with a small molecule inducer that binds to the chemically inducible promoter. In some embodiments, the small molecule inducer is selected from the group consisting of lactose, arabinose, and doxycycline.

### BRIEF DESCRIPTION OF DRAWINGS

FIG. 1 shows a plasmid schematic for transient transfection plasmids. Degradation domains (DD) are appended to the AAV pHelper and pRepCap plasmid. The EGFP-expressing transfer plasmid is not modified.

FIG. 2 shows a plasmid schematic for stable integration plasmids. Degradation domains are similarly appended to AAV pHelper and pRepCap. Sleeping Beauty IR/DRs and antibiotic selection cassettes are added to enhance the integration efficiency and enable efficient selection of cells with genomic integration events, respectively.

FIG. 3 shows FKBP and ecDHFR degradation domains were tested as fusions to AAV helper genes. ecDHFR degradation domain (ecDD) linked to E2A via N-terminal fusion and ecDHFR degradation domain linked to E4orf6 via N-terminal fusion were chosen as the best candidates tested for inducing helper genes using small molecules (22-fold and 1.8-fold induction respectively with TMP compared to without).

FIG. 4 shows the addition of ecDHFR degradation domains to both helper genes simultaneously maintained the induction of AAV titers observed for degradation domains linked to helper genes individually (45-fold induction for ecDHFR degradation domains linked to E2A and E4orf6 both via the N-terminus).

FIG. 5 shows the addition of ecDHFR degradation domains to the N-terminus of AAV2 Rep using different linker sequences shows inducibility upon TMP addition, except for the mCherry linker. mCherry, pGEX-2T and EAAAK (SEQ ID NO: 89) linkers were selected as the most interesting candidates for further testing, with 1-fold, 7.9-fold, and 7.8-fold induction respectively. Maximal AAV titers are reduced by addition of ecDHFR ecDDs to Rep in all cases.

FIG. 6 shows combinations of three ecDHFR degradation domains to E2A, E4, and Rep maintain inducibility of AAV titers. The reduction in maximal AAV titers is expected and is likely due linking the ecDD to Rep.

FIG. 7 shows a plasmid schematic for transient transfection plasmids. Constitutively expressed RfxCas13d (or alternatively DiCas7-11) complexes with crRNAs to bind and cleave the mRNA of Rep, E2A, and E4orf6.

FIG. 8 shows production of AAV with RfxCas13d and individual crRNAs targeting Rep, E2A, or E4orf6 reduce AAV titers up to 1000-fold.

FIG. 9 shows a plasmid schematic for shRNA knockdown of AAV-related genes. shRNAs are generated from a PolII promoter and downregulate the short Rep proteins, helper genes, or the gene of interest to be packaged.

FIG. 10 shows AAV infectious titers with and without shRNAs. Addition of shRNAs resulted in several fold reductions in AAV titers for all genes except Rep40 and EGFP, though no reduction in titers from EGFP was expected. Fold-reduction in AAV titers relative to wild type were approximately: shE2A=2.8, shE4-3.2, shRep40=1.3, shVP3=14.0, shEGFP=1.1, all shRNAs=5.5.

### DETAILED DESCRIPTION OF INVENTION

Viral vectors are a promising gene delivery modality for cell and gene therapy. The production of viral vectors normally entails transient transfection of plasmids into cell culture. However, stable integration of genes necessary to produce therapeutic viral vectors into the genome offers several advantages compared to traditional production via transient transfection. Since cells amplify the viral genes during their own cell division, large quantities of DNA and transfection reagent no longer need to be procured for the transfection process, reducing costs. Also, since the DNA is already within the nucleus, viral titers may be higher and more consistent due to minimal numbers of ‘untransfected’ cells and reduced variation associated with transfection steps. The simpler production process also saves scientist time.

However, several genes required for adeno-associated viral (AAV) vector production have been demonstrated by others to be cytostatic or cytotoxic, namely Rep, E2A and E4. The cytotoxic and cytostatic nature of these proteins has hampered the development of stable AAV producer cell lines in the widely used HEK293 cell line, since the native expression of adenovirus E1 genes in HEK293 cells upregulates expression of these toxic genes. Cells stably transfected with these genes fail to survive selection steps or have silenced expression, resulting in an inability to produce relevant quantities of AAV.

### I. Adeno-Associated Virus Production Systems

In some aspects, the disclosure relates to adeno-associated virus (AAV) production systems. In some embodiments, AAV production systems allow for inducible control of a gene product(s) required for AAV production, including a product(s) that is cytotoxic or cytostatic to a cell. This inducible control can be mediated at the transcriptional level (i.e., inducible control of mRNA concentration) or at the post-translational level (inducible control of protein expression).

An AAV production system, as described herein, comprises one or more nucleic acid molecules collectively comprising: (a) an AAV production component and (b) an expression control component.

### A. AAV Production Component

In some embodiments, the AAV production component, as described herein, comprises one or more nucleic acid molecules that collectively encode the gene products required for generation of an AAV in a recombinant host cell (or an “engineered cell” as described herein). Exemplary AAV gene products include Rep52, Rep40, Rep78, Rep68, E2A, E4Orf6, VARNA, CAP (VP1, VP2, VP3), AAP, and MAAP or a functional variant thereof. The Rep gene products (comprising Rep52, Rep40, Rep78 and Rep68) are involved in AAV genome replication. The E2A gene product is involved in aiding DNA synthesis processivity during AAV replication. The E4Orf6 gene product supports AAV replication. The VARNA gene product plays a role in regulating translation. The CAP gene products (comprising VP1, VP2, VP3) encode viral capsid proteins. The AAP gene product plays a role in capsid assembly. MAAP is a frameshifted VP1 protein and appears to play a role in the viral capsid as described in Ogden et al. Science 366.6469 (2019): 1139-1143, which is incorporated by reference in its entirety.

In some embodiments, an AAV production system comprises one or more nucleic acid sequences that collectively encode the gene products: Rep52 or Rep40; Rep78 or Rep68; E2A; E4Orf6; VARNA; VP1; VP2; VP3; and AAP. In some embodiments, an AAV production system comprises one or more nucleic acid sequences that collectively encode the gene products: Rep52, Rep40, Rep78, Rep68, E2A, E4Orf6, VARNA, VP1, VP2, VP3, and AAP. In some embodiments, the one or more nucleic acid molecules that collectively encode the gene products required for generation of an AAV are each operably linked to a promoter as described herein.

As used herein, the term “promoter” refers to a nucleic acid sequence that is bound by proteins to initiate transcription of RNA from DNA. A promoter may be a constitutive promoter (i.e., an unregulated promoter that allows for continual transcription). Examples of constitutive promoters are known in the art and include, but are not limited to, cytomegalovirus (CMV) promoters, elongation factor 1 α (EF1α) promoters, simian vacuolating virus 40 (SV40) promoters, ubiquitin-C (UBC) promoters, U6 promoters, p5 promoters, p19 promoters, p40 promoters, E2A promoters, E4 promoters and phosphoglycerate kinase (PGK) promoters. See e.g., Ferreira et al. Proc. Natl. Acad. Sci. U.S.A. 2013 July; 110(28): 11284-89; Pub. No.: US 2014/377861 A1; Qin et al. PloS one 5.5 (2010): e10611—the entireties of which are incorporated herein by reference. Alternatively, a promoter may be an inducible promoter (i.e., only activates transcription under specific circumstances). An inducible promoter may be a chemically inducible promoter, a temperature inducible promoter, or a light inducible promoter. Examples of inducible promoters are known in the art and include, but are not limited to, tetracycline/doxycycline inducible promoters, cumate inducible promoters, ABA inducible promoters, CRY2-CIB1 inducible promoters, DAPG inducible promoters, pTRE3G promoters, pTREtight promoters, the Gal4 UAS operator sequences and mifepristone inducible promoters, and a promoters containing at least one of VanR, TtgR, PhIF, or CymR operator sequences. See e.g., Stanton et al., ACS Synth. Biol. 2014 Dec. 19; 3(12): 880-91; Liang et al., Sci. Signal. 2011 Mar. 15; 4(164): rs2; Patent No.: U.S. Pat. No. 7,745,592 B2; Patent No.: U.S. Pat. No. 7,935,788 B2—the entireties of which are incorporated herein by reference.

In some embodiments, the AAV production component comprises a nucleic acid sequence encoding a functional variant of Rep52 comprising at least 80% (e.g., at least 80%, at least 85%, at least 90%, at least 95%, or at least 99%) identity to the amino acid sequence of SEQ ID NO: 6, wherein the functional variant is capable of functioning in AAV genome replication. In some embodiments, the AAV production component comprises a nucleic acid sequence encoding a Rep52 polypeptide comprising the amino acid sequence of SEQ ID NO: 6 operably linked to a promoter (as described herein).

In some embodiments, the AAV production component comprises a nucleic acid sequence encoding a functional variant of Rep40 comprising at least 80% (e.g., at least 80%, at least 85%, at least 90%, at least 95%, or at least 99%) identity to the amino acid sequence of SEQ ID NO: 7, wherein the functional variant is capable of functioning in AAV genome replication. In some embodiments, the AAV production component comprises a nucleic acid sequence encoding a Rep40 polypeptide comprising the amino acid sequence of SEQ ID NO: 7 operably linked to a promoter (as described herein).

In some embodiments, the AAV production component comprises a nucleic acid sequence encoding a functional variant of Rep78 comprising at least 80% (e.g., at least 80%, at least 85%, at least 90%, at least 95%, or at least 99%) identity to the amino acid sequence of SEQ ID NO: 8, wherein the functional variant is capable of functioning in AAV genome replication. In some embodiments, the AAV production component comprises a nucleic acid sequence encoding a Rep78 polypeptide comprising the amino acid sequence of SEQ ID NO: 8 operably linked to a promoter (as described herein).

In some embodiments, the AAV production component comprises a nucleic acid sequence encoding a functional variant of Rep68 comprising at least 80% (e.g., at least 80%, at least 85%, at least 90%, at least 95%, or at least 99%) identity to the amino acid sequence of SEQ ID NO: 9, wherein the functional variant is capable of functioning in AAV genome replication. In some embodiments, the AAV production component comprises a nucleic acid sequence encoding a Rep68 polypeptide comprising the amino acid sequence of SEQ ID NO: 9 operably linked to a promoter (as described herein).

In some embodiments, the AAV production component comprises a nucleic acid sequence encoding a functional variant of E2A comprising at least 80% (e.g., at least 80%, at least 85%, at least 90%, at least 95%, or at least 99%) identity to the amino acid sequence of SEQ ID NO: 10, wherein the functional variant is capable of aiding DNA synthesis processivity during AAV replication. In some embodiments, the AAV production component comprises a nucleic acid sequence encoding a E2A polypeptide comprising the amino acid sequence of SEQ ID NO: 10 operably linked to a promoter (as described herein).

In some embodiments, the AAV production component comprises a nucleic acid sequence encoding a functional variant of E4ORF6 comprising at least 80% (e.g., at least 80%, at least 85%, at least 90%, at least 95%, or at least 99%) identity to the amino acid sequence of SEQ ID NO: 11, wherein the functional variant is capable of supporting AAV replication. In some embodiments, the AAV production component comprises a nucleic acid sequence encoding a functional variant of E4ORF6 comprising at least 80% (e.g., at least 80%, at least 85%, at least 90%, at least 95%, or at least 99%) identity to the nucleic acid sequence of SEQ ID NO: 12, wherein the functional variant is capable of supporting AAV replication. In some embodiments, the AAV production component comprises a nucleic acid sequence encoding a E4ORF6 polypeptide comprising the amino acid sequence of SEQ ID NO: 11 operably linked to a promoter (as described herein). In some embodiments, the AAV production component comprises a nucleic acid sequence encoding SEQ ID NO: 12 operably linked to a promoter (as described herein).

In some embodiments, the AAV production component comprises a nucleic acid sequence encoding a functional variant of VARNA comprising at least 80% (e.g., at least 80%, at least 85%, at least 90%, at least 95%, or at least 99%) identity to the nucleic acid sequence of SEQ ID NO: 13, wherein the functional variant is capable regulating translation. In some embodiments, the AAV production component comprises a nucleic acid sequence encoding a VARNA of SEQ ID NO: 13 operably linked to a promoter (as described herein).

In some embodiments, the AAV production component comprises a nucleic acid sequence encoding a functional variant of VP1 comprising at least 80% (e.g., at least 80%, at least 85%, at least 90%, at least 95%, or at least 99%) identity to the amino acid sequence of SEQ ID NO: 14, wherein the functional variant is capable of being incorporated into the AAV capsid. In some embodiments, the AAV production component comprises a nucleic acid sequence encoding a VP1 polypeptide comprising the amino acid sequence of SEQ ID NO: 14 operably linked to a promoter (as described herein).

In some embodiments, the AAV production component comprises a nucleic acid sequence encoding a functional variant of VP2 comprising at least 80% (e.g., at least 80%, at least 85%, at least 90%, at least 95%, or at least 99%) identity to the amino acid sequence of SEQ ID NO: 15, wherein the functional variant is capable of being incorporated into the AAV capsid. In some embodiments, the AAV production component comprises a nucleic acid sequence encoding a VP2 polypeptide comprising the amino acid sequence of SEQ ID NO: 15 operably linked to a promoter (as described herein).

In some embodiments, the AAV production component comprises a nucleic acid sequence encoding a functional variant of VP3 comprising at least 80% (e.g., at least 80%, at least 85%, at least 90%, at least 95%, or at least 99%) identity to the amino acid sequence of SEQ ID NO: 16, wherein the functional variant is capable of being incorporated into the AAV capsid. In some embodiments, the AAV production component comprises a nucleic acid sequence encoding a VP3 polypeptide comprising the amino acid sequence of SEQ ID NO: 16 operably linked to a promoter (as described herein).

In some embodiments, the AAV production component comprises a nucleic acid sequence encoding a functional variant of AAP comprising at least 80% (e.g., at least 80%, at least 85%, at least 90%, at least 95%, or at least 99%) identity to the amino acid sequence of SEQ ID NO: 17, wherein the functional variant is capable of regulating AAV capsid assembly. In some embodiments, the AAV production component comprises a nucleic acid sequence encoding an AAP polypeptide comprising the amino acid sequence of SEQ ID NO: 17 operably linked to a promoter (as described herein).

In some embodiments, the AAV production component comprises a nucleic acid sequence encoding a functional variant of MAAP comprising at least 80% (e.g., at least 80%, at least 85%, at least 90%, at least 95%, or at least 99%) identity to the amino acid sequence of SEQ ID NO: 80, wherein the functional variant is capable of regulating AAV capsid assembly. In some embodiments, the AAV production component comprises a nucleic acid sequence encoding an AAP polypeptide comprising the amino acid sequence of SEQ ID NO: 80 operably linked to a promoter (as described herein).

In some embodiments, the AAV production component is (i.e., the gene products of the AAV component are) encoded on a single nucleic acid molecule. In other embodiments, multiple nucleic acid molecules collectively comprise the AAV production component (i.e., at least two of the gene products of the AAV production component are encoded on different nucleic acid molecules). For example, an AAV production component may comprise at least 2, at least 3, at least 4, at least 5, at least 6, at least 7, at least 8, at least 10, or at least 11 nucleic acid molecules. In some embodiments, an AAV production component comprises 2, 3, 4, 5, 6, 7, 8, 9, 10, or 11 nucleic acid molecules.

### B. The Expression Control Component

In some embodiments, the expression control component comprises a system for regulating the expression of a gene required for AAV production. In some embodiments, the expression control component can comprise a degradation domain component, a CRISPR component, an RNAi component, or a combination thereof, each capable of inducibly controlling the expression of one or more genes required for AAV production.

In some embodiments, the expression control component as described below is encoded on a single nucleic acid molecule. In other embodiments, multiple nucleic acid molecules collectively comprise the expression control component. For example, an expression control component may comprise at least 2, at least 3, at least 4, at least 5, at least 6, at least 7, at least 8, at least 10, or at least 11 nucleic acid molecules. In some embodiments, an expression control component comprises 2, 3, 4, 5, 6, 7, 8, 9, 10, 11 or more nucleic acid molecules.

### 1. Degradation Domain Components

In some embodiments, the expression control component, as described herein, comprises a nucleic acid molecule encoding a molecule binding degradation domain. In some embodiments, the expression control component comprises one or more (e.g. 1, 2, 3, 4, 5, 6, 7, 8, 9, 10, or 11) nucleic acid molecules each encoding a molecule binding degradation domain. In some embodiments, the one or more nucleic acid sequences encoding the molecule binding degradation domains are linked using a linker to one or more of the nucleic acid molecule sequences comprised within the AAV production component as described herein.

As described herein, a “molecule binding degradation domain” or “degradation domain” refers to a protein or peptide domain whose cellular degradation is decreased or increased by a specified molecule (e.g., a small molecule or a protein) binding to the degradation domain. In some embodiments, binding of the molecule to the degradation domain decreases degradation of the degradation domain. Exemplary molecule binding degradation domains include but are not limited to ecDHFR, FKBP, ornithine decarboxylase (ODC), modified ornithine decarboxylase (M-ODC), SMASH-tag, SURF system, Auxin system, and ligand induced degradation (LID) systems. Degradation domains are described in Trauth, Jonathan, et al. ACS Omega 4.2 (2019): 2766-2778, which is incorporated by reference in its entirety. In some embodiments, binding of the FKBP domain with Shield1, synthetic ligand of FKBP SLF*, or Shield2 reduces degradation of the FKBP domain as described in Grimley et al. Bioorganic & medicinal chemistry letters 18.2 (2008): 759-761, which is incorporated by reference in its entirety. Additional small molecules that bind the FKBP domain are described in Madsen, Daniel, et al. ACS combinatorial science 22.3 (2020): 156-164, which is incorporated by reference in its entirety. In some embodiments, binding of the ecDHFR domain with trimethoprim (TMP) reduces degradation of the ecDHFR domain. The ecDHFR degradation domain is described in Iwamoto, Mari, et al. Chemistry & biology 17.9 (2010): 981-988, which is incorporated by reference in its entirety. In some embodiments, molecule binding to the degradation domain increases degradation of the target protein (e.g., Cas13 or Cas7-11). For example, the Auxin system, Auxin treatment inducing degradation of the auxin-inducible degron. In another example, ornithine decarboxylase (ODC) or modified ornithine decarboxylase (M-ODC) degradation domain, when bound by antizyme, induces degradation of the ODC or M-ODC domain.

In some embodiments, the ecDHFR degradation domain comprises the amino acid sequence of SEQ ID NO: 81. In some embodiments, the ecDHFR degradation domain consists of the amino acid sequence of SEQ ID NO: 81. In some embodiments, the ODC degradation domain comprises an amino acids sequence having at least 80% (e.g., at least 80%, at least 85%, at least 90%, at least 95%, or at least 99%) identity to the amino acid sequence of SEQ ID NO: 62, wherein the degradation domain, when linked to a protein (e.g. the Rep52 protein), is capable of increasing the degradation rate of the protein. In some embodiments, the ODC degradation domain comprises the amino acid sequence of SEQ ID NO: 62. In some embodiments, the M-ODC degradation domain comprises an amino acid sequence having at least 80% (e.g., at least 80%, at least 85%, at least 90%, at least 95%, or at least 99%) identity to the amino acid sequence of any one of SEQ ID NO: 63-66, wherein the degradation domain, when linked to a protein (e.g. the Rep52 protein), is capable of increasing the degradation rate of the protein and wherein the M-ODC polypeptide comprises any one of the following mutations: D12A, T15A, C20A, and S24A. In some embodiments, the M-ODC degradation domain comprises the amino acid sequence of any one of SEQ ID NO: 63-66. In some embodiments, the M-ODC degradation domain comprises the amino acid sequence of any one of SEQ ID NO: 63-66.

In some embodiments, the degradation domain fused directly or indirectly to an amino acid sequence (e.g. a polypeptide required for AAV production or Cas13). In some embodiments, the degradation domain is fused to the N-terminal of an amino acid sequence. In some embodiments, the degradation domain is fused to the C-terminal of an amino acid sequence. In some embodiments, a degradation domain is fused to the N-terminal and C-terminal of an amino acid sequence.

As described herein, the term “linker” refers to an amino acid sequence that binds together or links two additional amino acid sequences. In some embodiments, the degradation domain is indirectly fused to an amino acid sequence (as described herein) using a linker. In some embodiments, the two additional amino acid sequences are proteins. In some embodiments, the linker is encoded between two protein amino acid sequences (e.g. encoded between a degradation domain and a protein required for AAV production). In a nonlimiting example, a linker could link an E2A protein to an ecDHFR degradation domain. In some embodiments, a nucleic acid sequence could encode from 5′ to 3′ an E2A protein, a linker, and a degradation domain. In some embodiments, a nucleic acid sequence could encode from 5′ to 3′ a degradation domain, a linker, and an E2A protein. Without being bound to theory, the linker allows proper spacing between the two proteins to allow each to fold and function. The length of an amino acid linker may vary. In some embodiments, an amino acid linker comprises at least 3 (e.g. at least 3, at least 4, at least 5, at least 6, at least 7, at least 8, at least 9, at least 10, at least 11, at least 12 at least 13, least 14, at least 15, at least 16, at least 17, at least 18, at least 19, at least 20, at least 25, at least 50, at least 100, at least 200, at least 300, or at least 400) amino acids. In some embodiments, an amino acid linker comprises 1, 2, 3, 4, 5, 6, 7, 8, 9, 10, 11, 12, 13, 14, 15, 16, 17, 18, 19, 20, 25, 50, 100, 200, 300, or 400 amino acids. In some embodiments, an amino acid linker comprises 2-4, 2-5, 2-6, 2-7, 2-8, 2-9, 2-10, 2-15, 2-25, 2-50, 3-4, 3-5, 3-6, 3-7, 3-8, 3-9, 3-10, 3-15, 3-25, 3-50, 3-100, 3-200, 3-300, 3-400, 5-7, 5-8, 5-9, 5-10, 5-15, 5-25, 5-50, 5-100, 5-200, 5-300, 5-400, 10-15, 10-25, 10-50, 10-100, 10-200, 10-300, 10-400, 25-50, 25-100, 25-200, 5-300, 25-400, 50-100, 50-200, 50-300, 50-400, 100-200, 100-300, 100-400 200-300, 200-400, 300-400 amino acids. Exemplary linkers include, but are not limited to GGGS (SEQ ID NO: 86), pMal, NAAAEF (SEQ ID NO: 87), GATRLPGS (SEQ ID NO: 88), mCherry, GSS, pGEX-2T, EAAAK (SEQ ID NO: 89), and AS linkers. Additional exemplary linkers include (GGGGS)₃ (SEQ ID NO: 90), (Gly): (SEQ ID NO: 91), (Gly)₆ (SEQ ID NO: 92), (EAAAK)ₙ (n=1-3) (SEQ ID NO: 93), A(EAAAK)₄ALEA(EAAAK)₄A (SEQ ID NO: 94), GGGGS (SEQ ID NO: 95), PAPAP (SEQ ID NO: 96), AEAAAKEAAAKA (SEQ ID NO: 97), (GGGGS)ₙ (n=1, 2, 4) (SEQ ID NO: 98), (Ala-Pro)ₙ (10-34 aa) (SEQ ID NO: 99), disulfide, VSQTSKLTR↓AETVFPDV (SEQ ID NO: 100), PLG↓LWA (SEQ ID NO: 101), RVL↓AEA (SEQ ID NO: 102); EDVVCC↓SMSY (SEQ ID NO: 103); GGIEGR↓GSc (SEQ ID NO: 104), TRHRQPR↓GWE (SEQ ID NO: 105), AGNRVRR↓SVG (SEQ ID NO: 106), RRRRRRR↓R↓R (SEQ ID NO: 107), GFLG↓ (SEQ ID NO: 108) and LE as described in Chen et al. Advanced drug delivery reviews 65.10 (2013): 1357-1369, which is incorporated by references herein it its entirety.

### 2. CRISPR Components

In some embodiments, the expression control component, comprises a CRISPR system capable of degrading the mRNA of one or more of the genes required for AAV production (as described in the AAV production component). In some embodiments, the expression control component comprises a nucleic acid molecule encoding a Cas13 protein and/or a Cas7-11 protein.

As used herein, the term “Cas13” may refer to any one CRISPR Cas protein of subtype VI or a functional variant thereof. In some embodiments, the Cas13 protein is selected from the group consisting of Cas13a, Cas13b, Cas13c, and Cas13d as described in Burmistrz et. al. International journal of molecular sciences 21.3 (2020): 1122, which is incorporated by reference in its entirety. In some embodiments, the Cas13 protein is a Cas13d protein selected from the group consisting of EsCas13d (Eubacterium siraeum), RspCas13d (Ruminococcus sp.), AdmCas13d (Anaerobic digester metagenome) and RfxCas13d (Ruminococcus flavefaciens) as described in Konermann et al. Cell 173.3 (2018): 665-676 and Yan et al. Molecular cell 70.2 (2018): 327-339 both of which are incorporated by reference in their entirety.

As used herein, the term “Cas7-11” may refer to CRISPR Cas subtype III-E or a functional variant thereof. In some embodiments, the Cas7-11 protein is DiCas7-11 (Desulfonema ishimotonii) as described in Özcan, et al. Nature 597.7878 (2021): 720-725, which is incorporated by reference in its entirety. In some embodiments, the Cas7-11 protein is selected from the group consisting of smCas7-11, omCas7-11, fmCas7-11, DpbaCas7-11, DiCas7-11, DsbaCas7-11, sstCas7-11, hvsCas7-11, hsmCas7-11, SybCas7-11, CbfCas7-11, CJcCas7-11, CsbCas7-11, hreCas7-11, hreCas7-11, CmaCas7-11, hvmCas7-11, gwCas7-11, wmCas7x3, NisCas7x3, gwCas7x3, hvmCas7x3, DsbCas7x3, DesCas7x3, MetCas7x3, MebCas7x3, GabCas7x3, or GamCas7x3, as described in Ozcan et al. Nature 597.7878 (2021): 720-725.

In some embodiments, the nucleic acid sequence encoding Cas13 and/or Cas7-11 as described above is operably linked to a promoter as described herein. In some embodiments, the nucleic acid sequence encoding Cas13 and/or Cas7-11 as described above is operably linked to a hEF1a promoter. In some embodiments, the nucleic acid sequence encoding for Cas13 and/or Cas7-11 is operably linked to a chemically inducible promoter as described herein. In some embodiments, the nucleic acid sequence encoding for Cas13 and/or Cas7-11 as described above is operably linked to a TRE3G chemically inducible promoter.

In some embodiments, the nucleic acid sequence encoding for a Cas13 and/or Cas7-11 is linked to a nucleic acid sequence encoding a degradation domain (as described herein). For example, in some embodiments, the nucleic acid sequence encoding for a Cas13 is linked to a nucleic acid sequence encoding a degradation domain, wherein the degradation domain is ODC or M-ODC (as described herein). In some embodiments, a polypeptide encoding Cas13 and a degradation domain comprises an amino acid sequence encoding from n-terminus to c-terminus: Cas13, a linker, and a degradation domain. In some embodiments, a polypeptide encoding Cas13 and a degradation domain comprises an amino acid sequence encoding from n-terminus to c-terminus: a degradation domain, a linker, and Cas13.

Similarly, in some embodiments, the nucleic acid sequence encoding for a Cas7-11 is linked to a nucleic acid sequence encoding a degradation domain, wherein the degradation domain is ODC or M-ODC (as described herein). In some embodiments, a polypeptide encoding Cas7-11 and a degradation domain comprises an amino acid sequence encoding from n-terminus to c-terminus: Cas7-11, a linker, and a degradation domain. In some embodiments, a polypeptide encoding Cas7-11 and a degradation domain comprises an amino acid sequence encoding from n-terminus to c-terminus: a degradation domain, a linker, and Cas7-11.

In some embodiments, the expression control component comprises a nucleic acid sequence encoding a RfxCas13d polypeptide or functional variant thereof capable of degrading RNA, wherein the RfxCas13d polypeptide or functional variant thereof comprises at least 80% (e.g., at least 80%, at least 85%, at least 90%, at least 95%, or at least 99%) identity to the amino acid sequence of SEQ ID NO: 28. In some embodiments, the expression control component comprises a nucleic acid sequence encoding a RfxCas13d polypeptide comprising the amino acid sequence of SEQ ID NO: 28. In some embodiments, the expression control component comprises a nucleic acid sequence encoding a RfxCas13d polypeptide consisting of the amino acid sequence of SEQ ID NO: 28.

In some embodiments, the expression control component comprises a nucleic acid sequence encoding a DiCas7-11 polypeptide or functional variant thereof capable of degrading RNA, wherein the DiCas7-11 polypeptide or functional variant thereof comprises at least 80% (e.g., at least 80%, at least 85%, at least 90%, at least 95%, or at least 99%) identity to the amino acid sequence of SEQ ID NO: 85. In some embodiments, the expression control component comprises a nucleic acid sequence encoding a DiCas7-11 polypeptide comprising the amino acid sequence of SEQ ID NO: 85. In some embodiments, the expression control component comprises a nucleic acid sequence encoding a DiCas7-11 polypeptide consisting of the amino acid sequence of SEQ ID NO: 85.

In some embodiments, the expression of the Cas13 and/or Cas7-11 is regulated by a RNAi as described below. For example, in some embodiments, expression of the Cas13 is regulated by one or more RNAi oligonucleotides each comprising a nucleic acid sequence sufficiently complementary to mRNA encoding Cas13 to direct RNAi-mediated degradation of the mRNA of Cas13. In some embodiments, the one or more RNAi oligonucleotides each comprise a nucleic acid sequence sufficiently complementary to mRNA that encodes SEQ ID NO: 28 to direct RNAi-mediated degradation of the mRNA. Similarly, in some embodiments, expression of the Cas7-11 is regulated by one or more RNAi oligonucleotides each comprising a nucleic acid sequence sufficiently complementary to mRNA encoding Cas7-11 to direct RNAi-mediated degradation of the mRNA of Cas7-11. In some embodiments, the one or more RNAi oligonucleotides each comprise a nucleic acid sequence sufficiently complementary to mRNA that encodes SEQ ID NO: 85 to direct RNAi-mediated degradation of the mRNA.

In some embodiments, the expression control component, as described herein, comprises one or more nucleic acid molecules encoding one or more guide RNAs. As described herein, the term “guide RNA” or “crRNA” refers to a RNA sequence capable of binding to and directing a Cas13 or a Cas7-11 to a target RNA sequence (e.g. mRNA encoding Rep52). In some embodiments, the term “crRNA” as refers to the DNA nucleic acid sequence encoding the crRNA. crRNAs comprise a nucleic acid sequence referred to as a spacer. In some embodiments, the spacer is about 15 to 84 base pairs in length and is sufficiently complementary to the target sequence (e.g. the RNA of Rep52) to direct the Cas13 to the target sequence. crRNAs are described in Cox et al. Science 358.6366 (2017): 1019-1027, which is incorporated by reference in its entirety. One of ordinary skill in the art would know how to design the nucleic acid sequence of the spacer to target the mRNA of a given gene, for example by using Cas13 design (https://cas13design.nygenome.org/). In some embodiments, the one or more crRNAs are sufficiently complementary to the mRNAs encoding each of the one or more genes required for AAV production (as described in the AAV production component) to direct a Cas13 or a Cas7-11 to degrade the mRNA. In some embodiments, a DNA nucleic acid sequence encoding one or more crRNA described herein is operably linked to a promoter (constitutive or inducible, as described herein). In some embodiments, the DNA nucleic acid sequence encoding any crRNA described herein is operably linked to a U6 promoter.

In some embodiments, the one or more crRNAs form a crRNA array. As used herein, the term “crRNA array” refers to a nucleic acid molecule operably linked to a promoter (constitutive or inducible as described herein) comprising a first nucleic acid sequence encoding each of one or more crRNAs and a second nucleic acid sequence one or more direct repeat (DR) regions. In some embodiments, DR regions are encoded both upstream and downstream of each crRNAs. As a nonlimiting example, the nucleic acid molecule encoding the crRNA array comprises the following general sequence: DR-crRNA-DR-crRNA-DR-crRNA-DR-crRNA-DR. In some embodiments, the crRNA array comprises 1, 2, 3, 4, 5, 6, 7, 8, 9, 10, 11, 12, 13, 14, 15, 16, 17, 18, 19, 20 or more crRNAs. In some embodiments, the Cas13 (as described herein) is capable of excising the crRNA sequences from the crRNA array.

### 3. RNAi-Component

In some embodiments, the expression control component comprises one or more nucleic acid molecules encoding one or more RNA interference (RNAi) oligonucleotides capable of directing degradation of: (i) a mRNA encoding one or more of the genes required for AAV production (as described in the AAV production component); (ii) a mRNA encoding for a CRISPR Cas protein (as described in CRISPR Component); or (iii) a combination thereof. In some embodiments, the one or more nucleic acid molecules encode for 1, 2, 3, 4, 5, 6, 7, 8, 9, 10, 11, 12, 13, 14, 15, 16, 17, 18, 19, 20 or more RNAi oligonucleotides. In some embodiments, the one or more RNAi oligonucleotides are selected from the group consisting of small interfering RNAs (siRNAs), microRNAs, artificial microRNAs (amiRNAs), antagomirs, and short-hairpin RNAs (shRNAs). In some embodiments, the one or more RNAi oligonucleotides are shRNAs. In some embodiments, the shRNA comprises a pLKO.1, pSico pSicoRmiR-3G (based on miR-16-2), pSUPER or miR-E miR backbone as described in Brummelkamp et al. Science 296.5567 (2002): 550-553 and Bhaskaran et al. Nature protocols 14.12 (2019): 3538-3553, each of which is incorporated by reference in its entirety. In some embodiments, the miR backbone comprises a loop. In some embodiments, the loop may be 1, 2, 3, 4 5, 6, 7, 8, 9, 10 11, 12, 13, 14, 15, 16, 17, 18, 19, 20, 21, 22, 23, 24, 25, 26, 27, 28, 29, or 30 nucleotides long. In some embodiments, the loop may be at least 5 (e.g. at least 5, at least 6, at least 7, at least 8, at least 9, at least 10 at least 11, at least 12, at least 13, at least 14, at least at least 15, at least 16, at least 17, at least 18, at least 19, or at least 20 nucleotide long. In some embodiments, the loop may be 1-5, 5-10, 5-15, 5-20, 5-25, 5-30, 10-15, 10-20, 10-25, 10-30, 15-20, 15-25, 15-30, 20-25, or 20-30 nucleotides long. In some embodiments, the loop is represented by NNNNN in SEQ ID NOs: 67-78. However, it is to be understood that the “NNNNN” in SEQ ID NOs: 67-78 may represent a loop of any length described herein. In some embodiments, the shRNA backbone is based on a miR-E backbone. The miR-E backbone is described in Fellmann, Christof, et al. Cell reports 5.6 (2013): 1704-1713, which is incorporated by reference in its entirety. In some embodiments, the shRNA is placed in the 3′ UTR immediately downstream of a PolII-driven gene (Neo-TagBFP) and flanked by splicing donor and acceptor sequences along with a polypyrimidine tract.

In some embodiments, the one or more nucleic acid molecules encoding one or more RNA interference (RNAi) oligonucleotides, further comprise a selection marker (as described herein), optionally a Neo-Tag BFP selection marker. In some embodiments, the one or more nucleic acid molecules each encoding the one or more RNAi oligonucleotides is operably linked to a promoter (constitutive or inducible, as described herein). In some embodiments, the DNA nucleic acid sequence encoding any crRNA described herein is operably linked to a CMV promoter.

### C. Engineered Cells for AAV Production

In some embodiments, an AAV production system described herein further comprises an engineered cell. In some embodiments, the engineered cell may comprise any part (and any combination of parts) of the AAV production systems described herein.

For example, an engineered cell may comprise at least a portion of the AAV production component. For example, and as described above, an AAV production component may comprise multiple nucleic acid molecules. In such embodiments, an engineered cell comprises one or more of said multiple nucleic acid molecules—each of which may be located extra-chromosomally or stably integrated into the genome of the engineered cell. In some embodiments, an engineered cell comprises the entire AAV production component.

Alternatively, or in addition, an engineered cell may comprise the expression control component of the AAV production system. In such embodiments, an engineered cell comprises one or more of said multiple nucleic acid molecules—each of which may be located extra-chromosomally or stably integrated into the genome of the engineered cell. In some embodiments, an engineered cell comprises the entire expression control component.

In some embodiments, an AAV production system comprises: (a) an engineered cell comprising an AAV production component comprising one or more heterologous nucleic acid molecules that collectively encode the genes required for AAV production and (b) an expression control component capable of controlling expression of at least one gene required for AAV production.

As used herein, the term “stably integrated” refers to an exogenous nucleic acid sequence, nucleic acid molecule, construct, gene, or nucleic acid sequence that has been inserted into the genome of and organism (e.g. the engineered cell as described herein) and is passed on to future generations after cell division. It is to be understood that any nucleic acid sequence, nucleic acid molecule, construct, gene or nucleic acid sequence described herein may be stably integrated. In some embodiments, any nucleic acid sequence, nucleic acid molecule, construct gene or nucleic acid sequence may be integrated into the genome using random integration, targeted integration, or transposon-mediated integration. It is to be understood that any of the stably integrated nucleic acid molecules described herein may comprise IR/DR sequences that are capable of binding the Sleeping Beauty transposase. Stable integration using the Sleeping Beauty transposase is described in Mátés, Lajos, et al. Nature genetics 41.6 (2009): 753-761 which is incorporated by reference in its entirety. In some embodiments, a IR/DR sequence comprises a Sleeping Beauty 100X (SB100X) IR/DR.

### D. Selection Marker

As used herein, the term “selection marker” or refers to a protein that—when introduced into or expressed in a cell—confers a trait that is suitable for selection. As used herein, the term “selection cassette” refers to a nucleic acid sequence encoding a selection marker operably linked to a promoter (as described herein) and a terminator.

A selection marker may be a fluorescent protein. Examples of fluorescent proteins are known in the art (e.g., TagBFP, EBFP2, EGFP, EYFP, mKO2, or Sirius). See e.g., Patent No.: U.S. Pat. No. 5,874,304; Patent No.: EP 0969284 A1; Pub. No.: US 2010/167394 A—the entireties of which are incorporated here by reference.

Alternatively, or in addition, a selection marker may be an antibiotic resistance protein. Examples of antibiotic resistance proteins are known in the art (e.g., facilitating puromycin, hygromycin, neomycin, zeocin, blasticidin, or phleomycin selection). See e.g., Pub. No.: WO 1997/15668 A2; Pub. No.: WO 1997/43900 A1—the entireties of which are incorporated here by reference.

### E. Landing Pad

An engineered cell described herein may further comprise a landing pad. As used herein, the term “landing pad” refers to a heterologous nucleic acid molecule sequence that facilitates the targeted insertion of a “payload” sequence into a specific locus (or multiple loci) of the cell's genome. Accordingly, the landing pad is integrated into the genome of the cell. A fixed integration site is desirable to reduce the variability between experiments that may be caused by positional epigenetic effects or proximal regulatory elements. The ability to control payload copy number is also desirable to modulate expression levels of the payload without changing any genetic components.

In some embodiments, the landing pad is located at a safe harbor site in the genome of the engineered cell. As used herein, the term “safe harbor site” refers to a location in the genome where genes or genetic elements can be introduced without disrupting the expression or regulation of adjacent genes and/or adjacent genomic elements do not disrupt expression or regulation of the introduced genes or genetic elements. Examples of safe harbor sites are known to those having skill in the art and include, but are not limited to, AAVS1, ROSA26, COSMIC, H11, CCR5, and LiPS-A3S. See e.g., Gaidukov et al., Nucleic Acids Res. 2018 May 4; 46(8): 4072-4086; Patent No.: U.S. Pat. No. 8,980,579 B2; Patent No.: U.S. Pat. No. 10,017,786 B2; Patent No.: U.S. Pat. No. 9,932,607 B2; Pub. No.: US 2013/280222 A; Pub. No.: WO 2017/180669 A1—the entireties of which are incorporated herein. In some embodiments, the safe harbor site is a known site. In other embodiments, the safe harbor site is a previously undisclosed site. See “Methods of Identifying High-Expressing Genomic Loci and Uses Thereof” herein. In some embodiments, an engineered cell described herein comprises a landing pad that is integrated at a safe harbor locus selected from the group consisting of AAVS1, ROSA26, COSMIC, H11, CCR5, and LiPS-A3S.

In some embodiments, the engineered cell is derived from a HEK293 cell. In some embodiments, the engineered HEK293 cell comprises a landing pad that is integrated at a safe harbor locus selected from the group consisting of AAVS1, ROSA26, COSMIC, H11, CCR5, and LiPS-A3S.

Each of the landing pads described herein comprises at least one recombination site. Recombination sites for various integrases have been identified previously. For example, a landing pad may comprise recombination sites corresponding to a Bxb1 integrase, lambda-integrase, Cre recombinase, F1p recombinase, gamma-delta resolvase, Tn3 resolvase, C31 integrase, or R4 integrase. Exemplary recombination site sequences are known in the art (e.g., attP, attB, attR, attL, Lox, and Frt).

The landing pads described herein may comprise one or more expression cassettes.

### F. Transcriptional Activator

In some embodiments, the AAV production system further comprises a nucleic acid sequence encoding a transcriptional activator. In some embodiments, the transcriptional activator is selected from the group consisting of TetOn-3G, TetOn-V16, TetOff-Advanced, VanR-VP16, TtgR-VP16, PhIF-VP16, and the cumate-responsive transactivators cTA and rcTA. In some embodiments, the nucleic acid sequence encoding the transcriptional activator is fused to a selection marker. In some embodiments, the transcriptional activator is operably linked to a promoter. In some embodiments, the transcriptional activation is operably linked to a constitutively active promoter. In some embodiments, the transcriptional activation is operably linked to a hEFla promoter. In some embodiments, the transcriptional activator, when exposed to a small molecule inducer, induces the expression of corresponding chemically inducible promoters within the engineered cell. In some embodiments, the small molecule inducer is selected from the group consisting of doxycycline, vanillate, phloretin, rapamycin, abscisic acid, gibberellic acid acetoxymethyl ester, and cumate.

### II. AAV Production Systems Comprising a Degradation Domain

In some aspects, the present disclosure provides AAV production systems comprising one or more genes required for AAV production linked to a degradation domain for control of expression. In a nonlimiting example, the AAV production system may comprise a polypeptide encoding E2A linked to a polypeptide encoding an ecDHFR molecule binding degradation domain, which binds trimethoprim. When trimethoprim is absent, E2A linked to the molecule binding domain is degraded. When trimethoprim is present, the degradation domain is stabilized and E2A concentration increases, which promotes production of AAV.

In some embodiments, the AAV production system comprises a nucleic acid sequence encoding a gene required for AAV production that is linked to a degradation domain. The modifier “DD-” as used herein refers to a gene linked to a molecule binding degradation domain.

In some embodiments, the AAV production system comprises: a nucleic acid sequence encoding for DD-Rep40 operably linked to a nucleic acid sequence of a promoter (constitutive or inducible, as described herein); a nucleic acid sequence encoding for DD-Rep52 operably linked to a nucleic acid sequence of a promoter (constitutive or inducible, as described herein); a nucleic acid sequence encoding for DD-Rep78 operably linked to a nucleic acid sequence of a promoter (constitutive or inducible, as described herein); a nucleic acid sequence encoding for DD-Rep68 operably linked to a nucleic acid sequence of a promoter (constitutive or inducible, as described herein); a nucleic acid sequence encoding for DD-Rep operably linked to a nucleic acid sequence of a promoter (constitutive or inducible, as described herein); a nucleic acid sequence encoding for DD-VP1 operably linked to a nucleic acid sequence of a promoter (constitutive or inducible, as described herein); a nucleic acid sequence encoding for DD-VP2 operably linked to a nucleic acid sequence of a promoter (constitutive or inducible, as described herein); a nucleic acid sequence encoding for DD-VP3 operably linked to a nucleic acid sequence of a promoter (constitutive or inducible, as described herein); a nucleic acid sequence encoding for DD-E2A operably linked to a nucleic acid sequence of a promoter (constitutive or inducible, as described herein); and a nucleic acid sequence encoding for DD-E4Orf6 operably linked to a nucleic acid sequence of a promoter (constitutive or inducible, as described herein); or any combination thereof.

In some embodiments, the AAV production system comprises a nucleic acid sequence encoding for DD-Rep78 operably linked to a nucleic acid sequence of a promoter (constitutive or inducible, as described herein). In some embodiments, DD-Rep78 is operably linked to a p5 promoter. As used herein, the term “DD-Rep78” refers to a polypeptide encoding any Rep78 or functional variant thereof (as described above) further comprising a degradation domain. In some embodiments, DD-Rep78 further comprises a linker (as described herein) that links Rep78 to the degradation domain. In some embodiments, DD-Rep78 comprises a nucleic acid sequence comprising a mCherry, pGEX-2T, GSG or EAAAK (SEQ ID NO: 89) linker. In some embodiments, DD-Rep78 comprises a polypeptide encoding from n-terminus to c-terminus: Rep78, a linker, and a degradation domain (as described herein). In some embodiments, DD-Rep78 comprises a polypeptide encoding from n-terminus to c-terminus: a degradation domain, a linker, and Rep78. In some embodiments, DD-Rep78 comprises a nucleic acid sequence encoding a functional DD-Rep78 polypeptide comprising at least 80% (e.g., at least 80%, at least 85%, at least 90%, at least 95%, or at least 99%) identity to the amino acid sequence of any one of SEQ ID NOs: 22-23. In some embodiments, DD-Rep78 comprises a nucleic acid sequence encoding a functional Rep78 polypeptide comprising the amino acid sequence of any one of SEQ ID NOs: 22-23. In some embodiments, DD-Rep78 comprises a nucleic acid sequence encoding a functional Rep78 polypeptide consisting of the amino acid sequence of any one of SEQ ID NOs: 22-23.

In some embodiments, the AAV production system comprises a nucleic acid sequence encoding for DD-Rep68 operably linked to a nucleic acid sequence of a promoter (constitutive or inducible, as described herein). In some embodiments, DD-Rep68 is operably linked to a p5 promoter. As used herein, the term “DD-Rep68” refers to a polypeptide encoding any Rep68 or functional variant thereof (as described above) further comprising a degradation domain. In some embodiments, DD-Rep68 further comprises a linker (as described herein) that links Rep68 to the degradation domain. In some embodiments, DD-Rep68 comprises a nucleic acid sequence comprising a mCherry, pGEX-2T, GSG or EAAAK (SEQ ID NO: 89) linker. In some embodiments, DD-Rep68 comprises a polypeptide encoding from n-terminus to c-terminus: Rep68, a linker, and a degradation domain. In some embodiments, DD-Rep68 comprises a polypeptide encoding from n-terminus to c-terminus: a degradation domain, a linker, and Rep68. In some embodiments, DD-Rep68 comprises a nucleic acid sequence encoding a functional Rep68 polypeptide comprising at least 80% (e.g., at least 80%, at least 85%, at least 90%, at least 95%, or at least 99%) identity to the amino acid sequence of SEQ ID NO: 24-25. In some embodiments, DD-Rep68 comprises a nucleic acid sequence encoding a functional Rep68 polypeptide comprising the amino acid sequence of SEQ ID NO: 24-25. In some embodiments, DD-Rep68 comprises a nucleic acid sequence encoding a functional Rep68 polypeptide consisting of the amino acid sequence of SEQ ID NO: 24-25.

In some embodiments, the AAV production system comprises a nucleic acid sequence encoding for DD-Rep52 operably linked to a nucleic acid sequence of a promoter (constitutive or inducible, as described herein). In some embodiments, DD-Rep52 is operably linked to a p19 promoter. As used herein, the term “DD-Rep52” refers to a polypeptide encoding any Rep52 or functional variant thereof (as described above) further comprising a degradation domain. In some embodiments, DD-Rep52 further comprises a linker (as described herein) that links Rep52 to the degradation domain. In some embodiments, DD-Rep52 comprises a nucleic acid sequence comprising a mCherry, pGEX-2T, GSG or EAAAK (SEQ ID NO: 89) linker. In some embodiments, DD-Rep52 comprises a polypeptide encoding from n-terminus to c-terminus: Rep52, a linker, and a degradation domain. In some embodiments, DD-Rep52 comprises a polypeptide encoding from n-terminus to c-terminus: a degradation domain, a linker, and Rep52. In some embodiments, DD-Rep52 comprises a nucleic acid sequence encoding a functional Rep52 polypeptide comprising at least 80% (e.g., at least 80%, at least 85%, at least 90%, at least 95%, or at least 99%) identity to the amino acid sequence of SEQ ID NO: 82. In some embodiments, DD-Rep52 comprises a nucleic acid sequence encoding a functional Rep52 polypeptide comprising the amino acid sequence of SEQ ID NO: 82. In some embodiments, DD-Rep52 comprises a nucleic acid sequence encoding a functional Rep52 polypeptide consisting of the amino acid sequence of SEQ ID NO: 82.

In some embodiments, the AAV production system comprises a nucleic acid sequence encoding for DD-Rep40 operably linked to a nucleic acid sequence of a promoter (constitutive or inducible, as described herein). In some embodiments, DD-Rep40 is operably linked to a p19 promoter. As used herein, the term “DD-Rep40” refers to a polypeptide encoding any Rep40 or functional variant thereof (as described above) further comprising a degradation domain. In some embodiments, DD-Rep40 further comprises a linker (as described herein) that links Rep40 to the degradation domain. In some embodiments, DD-Rep40 comprises an amino acid comprising a mCherry, pGEX-2T, GSG or EAAAK (SEQ ID NO: 89) linker. In some embodiments, DD-Rep40 comprises a polypeptide encoding from n-terminus to c-terminus: Rep40, a linker, and a degradation domain. In some embodiments, DD-Rep40 comprises a polypeptide encoding from n-terminus to c-terminus: a degradation domain, a linker, and Rep40. In some embodiments, DD-Rep40 comprises a nucleic acid sequence encoding a functional Rep40 polypeptide comprising at least 80% (e.g., at least 80%, at least 85%, at least 90%, at least 95%, or at least 99%) identity to the amino acid sequence of SEQ ID NO: 83. In some embodiments, DD-Rep40 comprises a nucleic acid sequence encoding a functional Rep40 polypeptide comprising the amino acid sequence of SEQ ID NO: 83. In some embodiments, DD-Rep40 comprises a nucleic acid sequence encoding a functional Rep40 polypeptide consisting of the amino acid sequence of SEQ ID NO: 83.

In some embodiments, the AAV production system comprises a nucleic acid sequence encoding for DD-E2A operably linked to a nucleic acid sequence of a promoter (constitutive or inducible, as described herein). In some embodiments, DD-E2A is operably linked to a E2A promoter. As used herein, the term “DD-E2A” refers to a polypeptide encoding any E2A or functional variant thereof (as described above) further comprising a degradation domain. In some embodiments, DD-E2A further comprises a linker (as described herein) that links E2A to the degradation domain. In some embodiments, DD-E2A comprises an amino acid comprising a mCherry, pGEX-2T, GSG or EAAAK (SEQ ID NO: 89) linker. In some embodiments, DD-E2A comprises a polypeptide encoding from n-terminus to c-terminus: E2A, a linker, and a degradation domain. In some embodiments, DD-E2A comprises a polypeptide encoding from n-terminus to c-terminus: a degradation domain, a linker, and E2A. In some embodiments, DD-E2A comprises a nucleic acid sequence encoding a functional E2A polypeptide comprising at least 80% (e.g., at least 80%, at least 85%, at least 90%, at least 95%, or at least 99%) identity to the amino acid sequence of SEQ ID NO: 26. In some embodiments, DD-E2A comprises a nucleic acid sequence encoding a functional E2A polypeptide comprising the amino acid sequence of SEQ ID NO: 26. In some embodiments, DD-E2A comprises a nucleic acid sequence encoding a functional E2A polypeptide consisting of the amino acid sequence of SEQ ID NO: 26.

In some embodiments, the AAV production system comprises a nucleic acid sequence encoding for DD-E4orf6 operably linked to a nucleic acid sequence of a promoter (constitutive or inducible, as described herein). In some embodiments, DD-E4orf6 is operably linked to an E4 promoter. As used herein, the term “DD-E4orf6” refers to a polypeptide encoding any E4orf6 or functional variant thereof (as described above) further comprising a degradation domain. In some embodiments, DD-E4orf6 further comprises a linker (as described herein) that links E4orf6 to the degradation domain. In some embodiments, DD-E4orf6 comprises an amino acid comprising a mCherry, pGEX-2T, GSG or EAAAK (SEQ ID NO: 89) linker. In some embodiments, DD-E4orf6 comprises a polypeptide encoding from n-terminus to c-terminus: E4orf6, a linker, and a degradation domain. In some embodiments, DD-E4orf6 comprises a polypeptide encoding from n-terminus to c-terminus: a degradation domain, a linker, and E4orf6. In some embodiments, DD-E4orf6 comprises a nucleic acid sequence encoding a functional E4orf6 polypeptide comprising at least 80% (e.g., at least 80%, at least 85%, at least 90%, at least 95%, or at least 99%) identity to the amino acid sequence of SEQ ID NO: 27. In some embodiments, DD-E4orf6 comprises a nucleic acid sequence encoding a functional E4orf6 polypeptide comprising the amino acid sequence of SEQ ID NO: 27. In some embodiments, DD-E4orf6 comprises a nucleic acid sequence encoding a functional E4orf6 polypeptide consisting of the amino acid sequence of SEQ ID NO: 27.

In some embodiments, the AAV production system comprises a nucleic acid sequence encoding for DD-Rep operably linked to a nucleic acid sequence of a promoter (constitutive or inducible, as described herein). As used herein, the term “DD-Rep” refers to nucleic acid sequence encoding any Rep or functional variant thereof (as described above) further comprising a degradation domain. In some embodiments, DD-Rep further comprises an amino acid encoding a linker (as described herein) that links Rep to the degradation domain. In some embodiments, DD-Rep comprises a nucleic acid sequence comprising a mCherry, pGEX-2T, GSG or EAAAK (SEQ ID NO: 89) linker. In some embodiments, DD-Rep comprises a polypeptide encoding from n-terminus to c-terminus: Rep, a linker, and a degradation domain. In some embodiments, DD-Rep comprises a polypeptide encoding from n-terminus to c-terminus: a degradation domain, a linker, and Rep.

In some embodiments, the AAV production system comprises a nucleic acid sequence encoding for DD-VP1 operably linked to a nucleic acid sequence of a promoter (constitutive or inducible, as described herein). In some embodiments, DD-VP1 is operably linked to a p40 promoter. As used herein, the term “DD-VP1” refers to a polypeptide encoding any VP1 or functional variant thereof (as described above) further comprising a degradation domain. In some embodiments, DD-VP1 further comprises a linker (as described herein) that links VP1 to the degradation domain. In some embodiments, DD-VP1 comprises an amino acid sequence comprising a mCherry, pGEX-2T, GSG or EAAAK (SEQ ID NO: 89) linker. In some embodiments, DD-VP1 comprises a polypeptide encoding from n-terminus to c-terminus: VP1, a linker, and a degradation domain. In some embodiments, DD-VP1 comprises a polypeptide encoding from n-terminus to c-terminus: a degradation domain, a linker, and VP1.

In some embodiments, the AAV production system comprises a nucleic acid sequence encoding for DD-VP2 operably linked to a nucleic acid sequence of a promoter (constitutive or inducible, as described herein). In some embodiments, DD-VP2 is operably linked to a p40 promoter. As used herein, the term “DD-VP2” refers to a polypeptide encoding any VP2 or functional variant thereof (as described above) further comprising a degradation domain. In some embodiments, DD-VP2 further comprises a linker (as described herein) that links VP2 to the degradation domain. In some embodiments, DD-VP2 comprises a nucleic acid sequence comprising a mCherry, pGEX-2T, GSG or EAAAK (SEQ ID NO: 89) linker. In some embodiments, DD-VP2 comprises a polypeptide encoding from n-terminus to c-terminus: VP2, a linker, and a degradation domain. In some embodiments, DD-VP2 comprises a polypeptide encoding from n-terminus to c-terminus: a degradation domain, a linker, and VP2.

In some embodiments, the AAV production system comprises a nucleic acid sequence encoding for DD-VP3 operably linked to a nucleic acid sequence of a promoter (constitutive or inducible, as described herein). In some embodiments, DD-VP3 is operably linked to a p40 promoter. As used herein, the term “DD-VP3” refers to a polypeptide encoding any VP3 or functional variant thereof (as described above) further comprising a degradation domain. In some embodiments, DD-VP3 further comprises a linker (as described herein) that links VP3 to the degradation domain. In some embodiments, DD-VP3 comprises a nucleic acid sequence comprising a mCherry, pGEX-2T, GSG or EAAAK (SEQ ID NO: 89) linker. In some embodiments, DD-VP3 comprises a polypeptide encoding from n-terminus to c-terminus: VP3, a linker, and a degradation domain. In some embodiments, DD-VP3 comprises a polypeptide encoding from n-terminus to c-terminus: a degradation domain, a linker, and VP3.

In some embodiments, the AAV production system comprises a nucleic acid sequence encoding for DD-AAP operably linked to a nucleic acid sequence of a promoter (constitutive or inducible, as described herein). In some embodiments, DD-AAP is operably linked to a p40 promoter. As used herein, the term “DD-AAP” refers to a polypeptide encoding any AAP or functional variant thereof (as described above) further comprising a degradation domain. In some embodiments, DD-AAP further comprises a linker (as described herein) that links AAP to the degradation domain. In some embodiments, DD-AAP comprises a nucleic acid sequence comprising a mCherry, pGEX-2T, GSG or EAAAK (SEQ ID NO: 89) linker. In some embodiments, DD-AAP comprises a polypeptide encoding from n-terminus to c-terminus: AAP, a linker, and a degradation domain. In some embodiments, DD-AAP comprises a polypeptide encoding from n-terminus to c-terminus: a degradation domain, a linker, and AAP. In some embodiments, DD-AAP comprises a nucleic acid sequence encoding a functional AAP polypeptide comprising at least 80% (e.g., at least 80%, at least 85%, at least 90%, at least 95%, or at least 99%) identity to the amino acid sequence of SEQ ID NO: 84. In some embodiments, DD-AAP comprises a nucleic acid sequence encoding a functional AAP polypeptide comprising the amino acid sequence of SEQ ID NO: 84. In some embodiments, DD-AAP comprises a nucleic acid sequence encoding a functional AAP polypeptide consisting of the amino acid sequence of SEQ ID NO: 84.

In some embodiments, the AAV production system comprises a nucleic acid sequence encoding for DD-MAAP operably linked to a nucleic acid sequence of a promoter (constitutive or inducible, as described herein). In some embodiments, DD-MAAP is operably linked to a p40 promoter. As used herein, the term “DD-MAAP” refers to a polypeptide encoding any MAAP or functional variant thereof (as described above) further comprising a degradation domain. In some embodiments, DD-MAAP further comprises a linker (as described herein) that links MAAP to the degradation domain. In some embodiments, DD-MAAP comprises a nucleic acid sequence comprising a mCherry, pGEX-2T, GSG or EAAAK (SEQ ID NO: 89) linker. In some embodiments, DD-MAAP comprises a polypeptide encoding from n-terminus to c-terminus: MAAP, a linker, and a degradation domain. In some embodiments, DD-MAAP comprises a polypeptide encoding from n-terminus to c-terminus: a degradation domain, a linker, and MAAP.

### III. Exemplary Embodiments of Engineered Cells Comprising Degradation Domains

In some aspects, the AAV production system further comprises an engineered cell for AAV production as described above. In some embodiments, the engineered cell comprises the one or more nucleic acid molecules collectively comprising: (a) an AAV production component as described above and (b) an expression control component comprising one or more degradation domains as described above. In some embodiments, the AAV production component and the expression control component are stably integrated into the genome of the engineered cell.

In some embodiments, each of the nucleic acid molecules of the AAV production system comprises a selection marker. In some embodiments, each nucleic acid molecule of the AAV production system comprises a nucleic acid sequence of a distinct selection marker.

### A. The First Stably Integrated Nucleic Acid Molecule

In some embodiments, the engineered cell comprises one or more stably integrated nucleic acid molecules. In some embodiments, the engineered cell comprises a first stably integrated nucleic acid molecule. In some embodiments, the first stably integrated nucleic acid molecule comprises the nucleic acid sequence encoding for Rep52 or Rep40. In some embodiments, the first stably integrated nucleic acid molecule comprises a nucleic acid sequence encoding for DD-Rep78 or DD-Rep68. In some embodiments, the first stably integrated nucleic acid molecule comprises a nucleic acid sequence encoding for VP1, a nucleic acid sequence encoding for VP2, a nucleic acid sequence encoding for VP3, and a nucleic acid sequence encoding for AAP. In some embodiments, the first stably integrated nucleic acid molecule comprises the nucleic acid sequence encoding for Rep52 or Rep40, the nucleic acid sequence encoding for DD-Rep78 or DD-Rep68, the nucleic acid sequence encoding for VP1, the nucleic acid sequence encoding for VP2, the nucleic acid sequence encoding for VP3, and the nucleic acid sequence encoding for AAP. In some embodiments, the first stably integrated nucleic acid molecule further comprises two IR/DR sequences that are capable of binding the Sleeping Beauty transposase. In some embodiments, the first stably integrated nucleic acid sequence further comprises a selection marker operably linked to a promoter as described herein.

In some embodiments, the first stably integrated nucleic acid molecule comprises nucleic acid sequences encoding for the amino acid sequences of SEQ ID NO: 6 or SEQ ID NO: 7; any one of SEQ ID NOs: 22-25; SEQ ID NOs: 14-17; and a selection marker.

In some embodiments, the first stably integrated nucleic acid molecule as described above has the same structure as depicted in the FIG. 2 diagram comprising Rep78/68.

### B. The Second Stably Integrated Nucleic Acid Molecule

In some embodiments, the engineered cell for AAV production comprising one or more stably integrated nucleic acid molecules comprises the first stably integrated nucleic acid molecule and a second stably integrated nucleic acid molecule. In some embodiments, the second stably integrated nucleic acid molecule comprises a nucleic acid sequence encoding for DD-E2A. In some embodiments, the second stably integrated nucleic acid molecule comprises a nucleic acid sequence encoding for DD-E4orf6. In some embodiments, the second stably integrated nucleic acid molecule comprises nucleic acid sequence encoding for VARNA. In some embodiments, the second stably integrated nucleic acid molecule comprises a nucleic acid sequence encoding for DD-E2A, a nucleic acid sequence encoding for DD-E4orf6, and a nucleic acid sequence encoding for VARNA. In some embodiments, the second stably integrated nucleic acid molecule further comprises two IR/DR sequences that are capable of binding the Sleeping Beauty transposase. In some embodiments, the second stably integrated nucleic acid sequence further comprises a selection marker operably linked to a promoter as described herein.

In some embodiments, the second stably integrated nucleic acid molecule comprises nucleic acid sequences encoding for the amino acid sequence of SEQ ID NO: 26. In some embodiments, the second stably integrated nucleic acid molecule comprising the nucleic acid sequences encoding for the amino acid sequence of SEQ ID NO: 26 further comprises a nucleic acid molecule encoding for the amino acid sequence of SEQ ID NO: 27. In some embodiments, the second stably integrated nucleic acid molecule comprising a nucleic acid sequence encoding for the amino acid sequence of each of SEQ ID NO: 26 and SEQ ID NO: 27 further comprises a nucleic acid molecule encoding for the amino acid sequence of SEQ ID NO: 13.

In some embodiments, the second stably integrated nucleic acid molecule as described above has the same structure as depicted in the FIG. 2 diagram comprising DD-E2A.

### C. The Third Stably Integrated Nucleic Acid Molecule

In some embodiments, the engineered cell for AAV production comprising one or more stably integrated nucleic acid molecules comprises a first stably integrated nucleic acid molecule as described above, a second stably integrated nucleic acid molecule as described above and comprises a third stably integrated nucleic acid molecule. In some embodiments, the third stably integrated nucleic acid molecule comprises a nucleic acid sequence encoding a payload (as described herein). In some embodiments, the payload is a fluorescent protein (e.g., eGFP). In some embodiments, the third stably integrated nucleic acid molecule comprises a selection cassette as described herein. In some embodiments, the third integrated nucleic acid molecule further comprises two IR/DR sequences that are capable of binding the Sleeping Beauty transposase.

In some embodiments, the second stably integrated nucleic acid molecule as described above has the same structure as depicted in the FIG. 2 diagram comprising eGFP.

### IV. Methods of Using Engineered Cells for AAV Production Comprising Degradation Domains.

In some aspects, the present disclosure provides methods for producing AAV using an AAV production system comprising one or more nucleic acid molecules collectively comprising: (a) an AAV production component and (b) an expression control component comprising one or more degradation domains as described herein. In some embodiments, the method of AAV production comprises transfecting or stably integrating into an engineered cell any combination of the one or more nucleic acid molecules collectively comprising the AAV production component and the expression control component as described herein. In some embodiments, the method of AAV production further comprises transfecting a nucleic acid molecule comprising a payload for AAV delivery (e.g. a therapeutic DNA sequence) as described above. In some embodiments, the engineered cell used in the method of AAV production is selected from any one of the engineered cells for AAV production comprising a degradation domain described herein. In some embodiments, the method comprises growing the engineered cell to a confluency that is optimal for AAV production. An optimal confluency may be dependent, for example, on the type of cell the engineered cell is derived from. The skilled person will know or be able to determine the optimal confluency for AAV production. In some embodiments, the method comprises culturing the engineered cell with a molecule capable of binding to and stabilizing the degradation domain to reduce degradation. In some embodiments, the method comprises harvesting the AAV produced from the culture of engineered cells using methods that are well known to those of skill in the art.

### V. AAV Production Systems Comprising a Cas13 or a Cas7-11

In some aspects, the present disclosure provides AAV production systems comprising one or more nucleic acid molecules encoding an AAV production component (as described above) and an expression control component comprising a nucleic acid sequence encoding a Cas13 and/or a Cas7-11 as described herein. In some embodiments, the Cas is RfxCas13d or DiCas7-11. In a nonlimiting example, the AAV production system may comprise a nucleic acid molecule encoding E2A, a nucleic acid molecule encoding RfxCas13d or DiCas7-11, and a nucleic acid molecule encoding for each of one or more RfxCas13d crRNAs or DiCas7-11 crRNAs, wherein the crRNAs comprise sequences that are sufficiently complementary to E2A mRNA to direct the RfxCas13d or DiCas7-11 to degrade the E2A mRNA. In some embodiments, the nucleic acid sequence encoding RfxCas13d or DiCas7-11 is operably linked to a chemically inducible promoter as described above. In some embodiments, the nucleic acid molecules encoding each of the one or more crRNAs is operably linked to a chemically inducible promoter as described above.

### A. Cas13 and Cas7-11

See section I(B)(2) for a description of Cas13 and Cas7-11.

### B. Cas13 and Cas7-11 Guide RNAs

In some embodiments, the expression control component comprises one or more nucleic acid sequences encoding for one or more crRNAs each comprising a nucleic acid sequence sufficiently complementary to the RNA (e.g. mRNA) encoding one or more of the genes required for AAV production (as described above) to direct Cas13 or DiCas7-11 to degrade the RNA of the one or more genes required for AAV production. In some embodiments, the expression control component comprises one or more nucleic acid sequence encoding crRNAs each comprising a nucleic acid sequence sufficiently complementary to collectively degrade the RNA encoding Rep52, Rep40, Rep68, Rep78, Rep, E2A, E4ORF6, VP1, VP2, VP3, VARNA and AAP, or any combination of these genes.

In some embodiments, the expression control component comprises one or more nucleic acid sequences encoding for one or more crRNAs each comprising a nucleic acid sequence sufficiently complementary to mRNA encoding Rep52 to direct Cas13 or DiCas7-11 to degrade the mRNA of Rep52. In some embodiments, the one or more crRNAs each comprise a nucleic acid sequence sufficiently complementary to mRNA that encodes SEQ ID NO: 6 to direct Cas13 or DiCas7-11 to degrade the mRNA. In some embodiments, one or more crRNAs of the expression control component comprise a nucleic acid sequence having at least 80% (e.g., at least 80%, at least 85%, at least 90%, at least 95%, or at least 99%) identity to any one of SEQ ID NO: 37-38. In some embodiments, one or more single guide RNAs each comprise a nucleic acid sequence comprising any one of SEQ ID NO: 37-38. In some embodiments, the one or more single guide RNAs each comprise a spacer that consists of any one of SEQ ID NO: 37-38.

In some embodiments, the expression control component comprises one or more nucleic acid sequences encoding for one or more crRNAs each comprising a nucleic acid sequence sufficiently complementary to mRNA encoding Rep40 to direct Cas13 or DiCas7-11 to degrade the mRNA of Rep40. In some embodiments, the one or more crRNAs each comprise a nucleic acid sequence sufficiently complementary to mRNA that encodes SEQ ID NO: 7 to direct Cas13 or DiCas7-11 to degrade the mRNA. In some embodiments, one or more crRNAs of the expression control component comprise a nucleic acid sequence having at least 80% (e.g., at least 80%, at least 85%, at least 90%, at least 95%, or at least 99%) identity to any one of SEQ ID NO: 33, 37, and 38. In some embodiments, one or more single guide RNAs each comprise a nucleic acid sequence comprising any one of SEQ ID NO: 33, 37, and 38. In some embodiments, the one or more single guide RNAs each comprise a spacer that consists of any one of SEQ ID NO: 33, 37, and 38.

In some embodiments, the expression control component comprises one or more nucleic acid sequences encoding for one or more crRNAs each comprising a nucleic acid sequence sufficiently complementary to mRNA encoding Rep78 to direct Cas13 or DiCas7-11 to degrade the mRNA of Rep78. In some embodiments, the one or more crRNAs each comprise a nucleic acid sequence sufficiently complementary to mRNA that encodes SEQ ID NO: 8 to direct Cas13 or DiCas7-11 to degrade the mRNA. In some embodiments, one or more crRNAs of the expression control component comprise a nucleic acid sequence having at least 80% (e.g., at least 80%, at least 85%, at least 90%, at least 95%, or at least 99%) identity to any one of SEQ ID NO: 34-38. In some embodiments, one or more single guide RNAs each comprise a nucleic acid sequence comprising any one of SEQ ID NO: 34-38. In some embodiments, the one or more single guide RNAs each comprise a spacer that consists of any one of SEQ ID NO: 34-38.

In some embodiments, the expression control component comprises one or more nucleic acid sequences encoding for one or more crRNAs each comprising a nucleic acid sequence sufficiently complementary to mRNA encoding Rep68 to direct Cas13 or DiCas7-11 to degrade the mRNA of Rep68. In some embodiments, the one or more crRNAs each comprise a nucleic acid sequence sufficiently complementary to mRNA that encodes SEQ ID NO: 9 to direct Cas13 or DiCas7-11 to degrade the mRNA. In some embodiments, one or more crRNAs of the expression control component comprise a nucleic acid sequence having at least 80% (e.g., at least 80%, at least 85%, at least 90%, at least 95%, or at least 99%) identity to any one of SEQ ID NO: 33, 37 and 38. In some embodiments, one or more single guide RNAs each comprise a nucleic acid sequence comprising any one of SEQ ID NO: 33, 37 and 38. In some embodiments, the one or more single guide RNAs each comprise a spacer that consists of any one of SEQ ID NO: 33, 37 and 38.

In some embodiments, the expression control component comprises one or more nucleic acid sequences encoding for one or more crRNAs each comprising a nucleic acid sequence sufficiently complementary to mRNA encoding Rep to direct Cas13 or DiCas7-11 to degrade the mRNA of Rep. In some embodiments, the one or more crRNAs each comprise a nucleic acid sequence sufficiently complementary to mRNA encoded by SEQ ID NO: 20 to direct Cas13 or DiCas7-11 to degrade the mRNA. In some embodiments, one or more crRNAs of the expression control component comprise a nucleic acid sequence having at least 80% (e.g., at least 80%, at least 85%, at least 90%, at least 95%, or at least 99%) identity to any one of SEQ ID NO: 30, and 33-38. In some embodiments, one or more single guide RNAs each comprise a nucleic acid sequence comprising any one of SEQ ID NO: 30, and 33-38. In some embodiments, the one or more single guide RNAs each comprise a spacer that consists of any one of SEQ ID NO: 30, and 33-38.

In some embodiments, the expression control component comprises one or more nucleic acid sequences encoding for one or more crRNAs each comprising a nucleic acid sequence sufficiently complementary to mRNA encoding E2A to direct Cas13 or DiCas7-11 to degrade the mRNA of E2A. In some embodiments, the one or more crRNAs each comprise a nucleic acid sequence sufficiently complementary to mRNA that encodes SEQ ID NO: 10 to direct Cas13 or DiCas7-11 to degrade the mRNA. In some embodiments, one or more crRNAs of the expression control component comprise a nucleic acid sequence having at least 80% (e.g., at least 80%, at least 85%, at least 90%, at least 95%, or at least 99%) identity to any one of SEQ ID NO: 29 or SEQ ID NO: 31. In some embodiments, one or more single guide RNAs each comprise a nucleic acid sequence comprising any one of SEQ ID NO: 29 or SEQ ID NO: 31. In some embodiments, the one or more single guide RNAs each comprise a spacer that consists of any one of SEQ ID NO: 29 or SEQ ID NO: 31.

In some embodiments, the expression control component comprises one or more nucleic acid sequences encoding for one or more crRNAs each comprising a nucleic acid sequence sufficiently complementary to mRNA encoding E4ORF6 to direct Cas13 or DiCas7-11 to degrade the mRNA of E4ORF6. In some embodiments, the one or more crRNAs each comprise a nucleic acid sequence sufficiently complementary to mRNA that encodes SEQ ID NO: 11 or is encoded by SEQ ID NO: 12 to direct Cas13 or DiCas7-11 to degrade the mRNA. In some embodiments, one or more crRNAs of the expression control component comprise a nucleic acid sequence having at least 80% (e.g., at least 80%, at least 85%, at least 90%, at least 95%, or at least 99%) identity to any one of SEQ ID NO: 32. In some embodiments, one or more single guide RNAs each comprise a nucleic acid sequence comprising any one of SEQ ID NO: 32. In some embodiments, the one or more single guide RNAs each comprise a spacer that consists of any one of SEQ ID NO: 32.

In some embodiments, the expression control component comprises one or more nucleic acid sequences encoding for one or more crRNAs each comprising a nucleic acid sequence sufficiently complementary to mRNA encoding VP1 to direct Cas13 or DiCas7-11 to degrade the mRNA of VP1. In some embodiments, the one or more crRNAs each comprise a nucleic acid sequence sufficiently complementary to mRNA that encodes SEQ ID NO: 14 to direct Cas13 or DiCas7-11 to degrade the mRNA.

In some embodiments, the expression control component comprises one or more nucleic acid sequences encoding for one or more crRNAs each comprising a nucleic acid sequence sufficiently complementary to mRNA encoding VP2 to direct Cas13 or DiCas7-11 to degrade the mRNA of VP2. In some embodiments, the one or more crRNAs each comprise a nucleic acid sequence sufficiently complementary to mRNA that encodes SEQ ID NO: 15 to direct Cas13 or DiCas7-11 to degrade the mRNA.

In some embodiments, the expression control component comprises one or more nucleic acid sequences encoding for one or more crRNAs each comprising a nucleic acid sequence sufficiently complementary to mRNA encoding VP3 to direct Cas13 or DiCas7-11 to degrade the mRNA of VP3. In some embodiments, the one or more crRNAs each comprise a nucleic acid sequence sufficiently complementary to mRNA that encodes SEQ ID NO: 16 to direct Cas13 or DiCas7-11 to degrade the mRNA.

In some embodiments, the expression control component comprises one or more nucleic acid sequences encoding for one or more crRNAs each comprising a nucleic acid sequence sufficiently complementary to mRNA encoding AAP to direct Cas13 or DiCas7-11 to degrade the mRNA of AAP. In some embodiments, the one or more crRNAs each comprise a nucleic acid sequence sufficiently complementary to mRNA that encodes SEQ ID NO: 17 to direct Cas13 or DiCas7-11 to degrade the mRNA.

In some embodiments, the expression control component comprises one or more nucleic acid sequences encoding for one or more crRNAs each comprising a nucleic acid sequence sufficiently complementary to mRNA encoding VARNA to direct Cas13 or DiCas7-11 to degrade the mRNA of VARNA. In some embodiments, the one or more crRNAs each comprise a nucleic acid sequence sufficiently complementary to mRNA encoded by SEQ ID NO: 13 to direct Cas13 or DiCas7-11 to degrade the mRNA.

In some embodiments, the expression control component comprises one or more nucleic acid sequences encoding for one or more crRNAs each comprising a nucleic acid sequence sufficiently complementary to mRNA encoding MAAP to direct Cas13 or DiCas7-11 to degrade the mRNA of MAAP. In some embodiments, the one or more crRNAs each comprise a nucleic acid sequence sufficiently complementary to mRNA that encodes SEQ ID NO: 80 to direct Cas13 or DiCas7-11 to degrade the mRNA.

### VI. Exemplary Embodiments of Engineered Cells Comprising CRISPR Components

In some aspects, the AAV production system further comprises an engineered cell for AAV production as described above. In some embodiments, the engineered cell comprises the one or more nucleic acid molecules collectively comprising: (a) an AAV production component as described above and (b) an expression control component comprising a Cas13 and/or a DiCas7-11 as described above. In some embodiments, the AAV production component and the expression control component are stably integrated into the genome of the engineered cell.

In some embodiments, the engineered cell further comprises one or more nucleic acid molecules encoding a transcriptional activator as described above. In some embodiments, the one or more nucleic acid molecules encoding a transcriptional activator as described above are stably integrated.

In some embodiments, each of the nucleic acid molecules of the AAV production system comprises a selection marker. In some embodiments, each nucleic acid molecule of the AAV production system comprises a nucleic acid sequence of a distinct selection marker.

### A. The First Stably Integrated Nucleic Acid Molecule

In some embodiments, the engineered cell comprises one or more stably integrated nucleic acid molecules. In some embodiments, the engineered cell comprises a first stably integrated nucleic acid molecule. In some embodiments, the first stably integrated nucleic acid molecule comprises a nucleic acid sequence encoding for a Cas13 or DiCas7-11 polypeptide as described herein. In some embodiments, the first stably integrated nucleic acid molecule comprises a nucleic acid sequence encoding for a Cas13d polypeptide as described herein. In some embodiments, the first stably integrated nucleic acid molecule comprises a nucleic acid sequence encoding for a RfxCas13d polypeptide as described herein. In some embodiments, the first stably integrated nucleic acid molecule comprises a nucleic acid sequence encoding for a Cas7-11 polypeptide as described herein. In some embodiments, the first stably integrated nucleic acid molecule comprises a nucleic acid sequence encoding for a DiCas7-11 polypeptide as described herein.

In some embodiments, the first stably integrated nucleic acid molecule further comprises a degradation domain as described herein. In some embodiments, the degradation domain is a domain (e.g., an auxin-inducible degron or a ODC or M-ODC degron) that is degraded when bound by a molecule, as described herein.

In some embodiments, the nucleic acid sequence encoding for a Cas13 polypeptide is operably linked to a promoter as described herein (constitutive or inducible).

In some embodiments, the first stably integrated nucleic acid molecule comprises a selection cassette as described herein.

In some embodiments, the first stably integrated nucleic acid molecule further comprises two IR/DR sequences that are capable of binding the Sleeping Beauty transposase.

In some embodiments, the first stably integrated nucleic acid molecule comprises a nucleic acid sequence encoding for a RfxCas13d polypeptide of SEQ ID NO: 28 operably linked to an inducible promoter as described herein.

In some embodiments, the first stably integrated nucleic acid molecule comprises a nucleic acid sequence encoding for a DiCas7-11 polypeptide of SEQ ID NO: 85 operably linked to an inducible promoter as described herein.

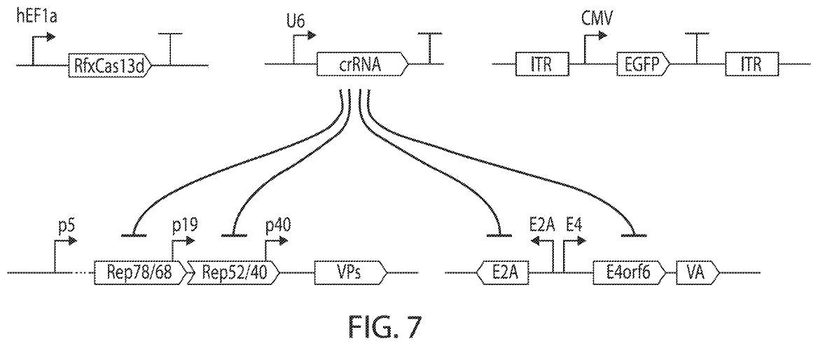

In some embodiments, the first stably integrated nucleic acid molecule as described above has the same structure as is depicted in the schematic of FIG. 7 comprising a RfxCas13d.

In some embodiments, the first stably integrated nucleic acid molecule as described above has the same structure as is depicted in the schematic of FIG. 7 comprising a DiCas7-11.

### B. The Second Stably Integrated Nucleic Acid Molecule

In some embodiments, the engineered cell for AAV production comprising one or more stably integrated nucleic acid molecules comprises the first stably integrated nucleic acid molecule and a second stably integrated nucleic acid molecule. In some embodiments, the second stably integrated nucleic acid molecule comprises a nucleic acid sequence encoding for each of one or more crRNAs as described herein. In some embodiments, the one or more crRNAs are operably linked to a promoter (constitutive or inducible) as described herein. In some embodiments, the one or more crRNAs are each operably linked to a U6 promoter.

In some embodiments, the one or more crRNAs each comprise a nucleic acid sequence sufficiently complementary to any one of the nucleic acid sequences encoding Rep52 or Rep40 to direct Cas13 or Cas7-11 to degrade the mRNA of Rep52 or Rep40 (as described herein).

In some embodiments, the one or more crRNAs each comprise a nucleic acid sequence sufficiently complementary to any one of the nucleic acid sequences encoding Rep78 or Rep68, to direct Cas13 or Cas7-11 to degrade the mRNA of Rep78 or Rep68 (as described herein).

In some embodiments, the one or more crRNAs each comprise a nucleic acid sequence sufficiently complementary to any one of the nucleic acid sequences encoding E2A, to direct Cas13 or Cas7-11 to degrade the mRNA of E2A (as described herein).

In some embodiments, the one or more crRNAs each comprise a nucleic acid sequence sufficiently complementary to any one of the nucleic acid sequences encoding E4Orf6 to direct Cas13 or Cas7-11 to degrade the mRNA of E4Orf6 (as described herein).

In some embodiments, the one or more crRNAs each comprise a nucleic acid sequence sufficiently complementary to any one of the nucleic acid sequences encoding Rep52 or Rep40, Rep78 or Rep68, E2A, and E4Orf6 to direct Cas13 or Cas7-11 to degrade the mRNA of Rep52 or Rep40, Rep78 or Rep68, E2A, and E4Orf6 (as described herein). In some embodiments, the second stably integrated nucleic acid molecule comprises a nucleic acid sequence encoding a crRNA array comprising the crRNAs that each comprise a nucleic acid sequence sufficiently complementary to any one of the nucleic acid sequences encoding Rep52 or Rep40, Rep78 or Rep68, E2A, and E4Orf6 to direct Cas13 or Cas7-11 to degrade the mRNA of Rep52 or Rep40, Rep78 or Rep68, E2A, and E4Orf6.

In some embodiments, the second stably integrated nucleic acid molecule comprises one or more nucleic acid sequences encoding each of one or more crRNAs that comprises a nucleic acid sequence encoding for SEQ ID NOs: 29-38 operably linked to a promoter (as described herein). In some embodiments, the second stably integrated nucleic acid molecule comprises one or more nucleic acid sequences encoding a crRNA array comprising any combination of SEQ ID NOs: 29-38.

In some embodiments, the second stably integrated nucleic acid molecule comprises a selection cassette as described herein.

In some embodiments, the second stably integrated nucleic acid molecule as described above has the same structure as is depicted in the schematic of FIG. 7 comprising crRNAs.

In some embodiments, the second stably integrated nucleic acid molecule further comprises two IR/DR sequences that are capable of binding the Sleeping Beauty transposase.

### C. The Third Stably Integrated Nucleic Acid Molecule

In some embodiments, the engineered cell for AAV production comprising one or more stably integrated nucleic acid molecules comprises a first stably integrated nucleic acid molecule as described above, a second stably integrated nucleic acid molecule as described above and comprises a third stably integrated nucleic acid molecule. In some embodiments, the third stably integrated nucleic acid molecule comprises one or more nucleic acid sequences collectively encoding all or any portion of the AAV production component (as described herein).

In some embodiments, the third stably integrated nucleic acid molecule comprises one or more nucleic acid sequence encoding for Rep52 or Rep40 (as described herein) operably linked to a promoter (as described herein).

In some embodiments, the third stably integrated nucleic acid molecule comprises one or more nucleic acid sequence encoding for Rep78 or Rep68 (as described herein) operably linked to a promoter (as described herein).

In some embodiments, the third stably integrated nucleic acid molecule comprises one or more nucleic acid sequence encoding for VP1, VP2, and VP3 (as described herein) operably linked to a promoter (as described herein).

In some embodiments, the third stably integrated nucleic acid molecule comprises a nucleic acid sequence encoding for AAP (as described herein) operably linked to a promoter (as described herein).

In some embodiments, the third stably integrated nucleic acid molecule comprises one or more nucleic acid sequence encoding for Rep52 or Rep40; Rep78 or Rep68; VP1, VP2, and VP3; and AAP (as described herein) each operably linked to a promoter (as described herein).

In some embodiments, the third stably integrated nucleic acid molecule comprises a selection cassette as described herein.

In some embodiments, the third stably integrated nucleic acid molecule further comprises two IR/DR sequences that are capable of binding the Sleeping Beauty transposase.

In some embodiments, the third stably integrated nucleic acid molecule as described above has the same structure as is depicted in the schematic of FIG. 7 comprising Rep52 or Rep40; Rep78 or Rep68; VP1, VP2, and VP3; and AAP.

### D. The Fourth Stably Integrated Nucleic Acid Molecule

In some embodiments, the engineered cell for AAV production comprising one or more stably integrated nucleic acid molecules comprises a first stably integrated nucleic acid molecule as described above, a second stably integrated nucleic acid molecule as described above, a third stably integrated nucleic acid molecule as described above and comprises a fourth stably integrated nucleic acid molecule. In some embodiments, the fourth stably integrated nucleic acid molecule comprises one or more nucleic acid sequences collectively encoding all or any portion of the AAV production component (as described herein).

In some embodiments, the fourth stably integrated nucleic acid molecule comprises a nucleic acid sequence encoding for E2A (as described herein) operably linked to a promoter (as described herein).

In some embodiments, the fourth stably integrated nucleic acid molecule comprises a nucleic acid sequence encoding for E4Orf6 (as described herein) operably linked to a promoter (as described herein).

In some embodiments, the fourth stably integrated nucleic acid molecule comprises a nucleic acid sequence encoding for VARNA (as described herein) operably linked to a promoter (as described herein).

In some embodiments, the fourth stably integrated nucleic acid molecule comprises one or more nucleic acid sequence encoding for E2A, E4Orf6, and VARNA (as described herein) each operably linked to a promoter (as described herein).

In some embodiments, the fourth stably integrated nucleic acid molecule comprises a selection cassette as described herein.

In some embodiments, the fourth stably integrated nucleic acid molecule further comprises two IR/DR sequences that are capable of binding the Sleeping Beauty transposase.

In some embodiments, the fourth stably integrated nucleic acid molecule as described above has the same structure as is depicted in the schematic of FIG. 7 comprising E2A, E4Orf6, and VARNA.

### E. The Fifth Stably Integrated Nucleic Acid Molecule

In some embodiments, the engineered cell for AAV production comprising one or more stably integrated nucleic acid molecules comprises a first stably integrated nucleic acid molecule as described above, a second stably integrated nucleic acid molecule as described above, a third stably integrated nucleic acid molecule as described above, a fourth stably integrated nucleic acid molecule as described above and comprises a fifth stably integrated nucleic acid molecule. In some embodiments, the fifth stably integrated nucleic acid molecule comprises a nucleic acid sequence encoding transcriptional activator as described above.

In some embodiments, the fifth stably integrated nucleic acid molecule further comprises two IR/DR sequences that are capable of binding the Sleeping Beauty transposase.

### F. The Sixth Stably Integrated Nucleic Acid Molecule

In some embodiments, the engineered cell for AAV production comprising one or more stably integrated nucleic acid molecules comprises a first stably integrated nucleic acid molecule as described above, a second stably integrated nucleic acid molecule as described above, a third stably integrated nucleic acid molecule as described above, a fourth stably integrated nucleic acid molecule as described above, a fifth stably integrated nucleic acid molecule as described above, and comprises a sixth stably integrated nucleic acid molecule. In some embodiments, the sixth stably integrated nucleic acid molecule comprises a nucleic acid sequence encoding each of a selection cassette operably linked to a promoter. In some embodiments, the sixth stably integrated nucleic acid molecule comprises a payload operably linked to a promoter. In some embodiments, the payload is a nucleic acid sequence encoding a fluorescent protein marker (as described herein), such as EGFP. In some embodiments, the sixth stably integrated nucleic acid molecule further comprises two inverted terminal repeat (ITR) sequences.

In some embodiments, the sixth stably integrated nucleic acid molecule further comprises two IR/DR sequences that are capable of binding the Sleeping Beauty transposase.

### VII. Methods of Using Engineered Cells for AAV Production Comprising Cas13 or Cas7-11.

In some aspects, the present disclosure provides methods for producing AAV using an AAV production system comprising one or more nucleic acid molecules collectively comprising: (a) an AAV production component and (b) an expression control component comprising Cas13 or Cas7-11 as described herein. In some embodiments, the method of AAV production comprises transfecting or stably integrating into an engineered cell any combination of the one or more nucleic acid molecules collectively comprising the AAV production component and the expression control component as described herein. In some embodiments, the method of AAV production further comprises transfecting a nucleic acid molecule comprising a payload for AAV delivery (e.g. a therapeutic DNA sequence) as described above. In some embodiments, the engineered cell used in the method of AAV production is selected from any one of the engineered cells for AAV production comprising a Cas13 or Cas7-11 described herein. In some embodiments, the method comprises growing the engineered cell to a confluency that is optimal for AAV production. An optimal confluency may be dependent, for example, on the type of cell the engineered cell is derived from. The skilled person will know or be able to determine the optimal confluency for AAV production. In some embodiments, the method comprises culturing the engineered cell with a small molecule inducer capable of inducing expression of the Cas13 polypeptide, the Cas7-11 polypeptide, and/or the one or more crRNAs (as described above), and then removing the small molecule inducer to promote AAV production. In some embodiments, the method comprises culturing the engineered cell with a molecule capable of decreasing expression of the Cas13 polypeptide or the Cas7-11 polypeptide by binding to a degradation domain fused to the Cas13 polypeptide or the Cas7-11 polypeptide, as described herein. In some embodiments, the method comprises harvesting the AAV produced from the culture of engineered cells using methods that are well known to those of skill in the art.

### VIII. AAV Production Systems Comprising Inhibitory RNAs

In some aspects, the present disclosure provides AAV production systems comprising one or more nucleic acid molecules encoding an AAV production component (as described above) and an expression control component comprising one or more nucleic acid sequences encoding one or more RNA interference (RNAi) oligonucleotides (as described herein). In some embodiments, the RNAi oligonucleotide is an shRNA. In a nonlimiting example, the AAV production system may comprise a nucleic acid molecule encoding E2A, and a nucleic acid molecule encoding for each of one or more shRNAs that are sufficiently complementary to E2A mRNA to direct RNAi mediated degradation of the E2A mRNA. In some embodiments, the nucleic acid sequence encoding the one or more inhibitory RNAi oligonucleotides is operably linked to a chemically inducible promoter as described above.

### A. Inhibitory RNA Oligonucleotides

In some embodiments, the expression control component comprises one or more nucleic acid sequences encoding for one or more RNAi oligonucleotides each comprising a nucleic acid sequence sufficiently complementary to the RNA (e.g. mRNA) encoding one or more of the genes required for AAV production (as described above) to direct RNAi-mediated degradation of RNA of the one or more genes required for AAV production. In some embodiments, the expression control component comprises one or more nucleic acid sequences encoding RNAi oligonucleotides each comprising a nucleic acid sequence sufficiently complementary to collectively degrade the RNA encoding Rep52, Rep40, Rep68, Rep78, Rep, E2A, E4ORF6, VP1, VP2, VP3, VARNA and AAP, or any combination of these genes. In some embodiments, the RNAi oligonucleotide is an shRNA. It is to be understood that any RNAi oligonucleotide described herein may be an shRNA.

In some embodiments, the expression control component comprises one or more nucleic acid sequences encoding for one or more RNAi oligonucleotides each comprising a nucleic acid sequence sufficiently complementary to mRNA encoding Rep52 to direct RNAi-mediated degradation of the mRNA of Rep52. In some embodiments, the one or more RNAi oligonucleotides each comprise a nucleic acid sequence sufficiently complementary to mRNA that encodes SEQ ID NO: 6 to direct RNAi-mediated degradation of the mRNA. In some embodiments, the one or more RNAi oligonucleotides sufficiently complementary to mRNA of Rep52 to direct RNAi-mediated degradation of the mRNA of Rep52 each comprise a nucleic acid sequence comprising at least 80% (e.g., at least 80%, at least 85%, at least 90%, at least 95%, or at least 99%) identity to any one of SEQ ID NOs: 45-47 and 73-75. In some embodiments, the one or more RNAi oligonucleotides each comprise a nucleic acid sequence comprising any one of SEQ ID NOs: 45-47 and 73-75. In some embodiments, the one or more RNAi oligonucleotides each comprise a spacer that consists of any one of SEQ ID NOs: 45-47 and 73-75.

In some embodiments, the expression control component comprises one or more nucleic acid sequences encoding for one or more RNAi oligonucleotides each comprising a nucleic acid sequence sufficiently complementary to mRNA encoding Rep40 to direct RNAi-mediated degradation of Rep40. In some embodiments, the one or more RNAi oligonucleotides each comprise a nucleic acid sequence sufficiently complementary to mRNA that encodes SEQ ID NO: 7 to direct RNAi-mediated degradation of the mRNA. In some embodiments, the one or more RNAi oligonucleotides sufficiently complementary to mRNA of Rep40 to direct RNAi-mediated degradation of Rep40 each comprise a nucleic acid sequence comprising at least 80% (e.g., at least 80%, at least 85%, at least 90%, at least 95%, or at least 99%) identity to any one of SEQ ID NOs: 45-47 and 73-75. In some embodiments, the one or more RNAi oligonucleotides each comprise a nucleic acid sequence comprising any one of SEQ ID NOs: 45-47 and 73-75. In some embodiments, the one or more RNAi oligonucleotides each comprise a spacer that consists of any one of SEQ ID NOs: 45-47 and 73-75.

In some embodiments, the expression control component comprises one or more nucleic acid sequences encoding for one or more RNAi oligonucleotides each comprising a nucleic acid sequence sufficiently complementary to mRNA encoding Rep78 to direct RNAi-mediated degradation of Rep78. In some embodiments, the one or more RNAi oligonucleotides each comprise a nucleic acid sequence sufficiently complementary to mRNA that encodes SEQ ID NO: 8 to direct RNAi-mediated degradation of the mRNA. In some embodiments, the one or more RNAi oligonucleotides sufficiently complementary to mRNA of Rep78 to direct RNAi-mediated degradation of Rep78 each comprise a nucleic acid sequence comprising at least 80% (e.g., at least 80%, at least 85%, at least 90%, at least 95%, or at least 99%) identity to any one of SEQ ID NOs: 45-47 and 73-75. In some embodiments, the one or more RNAi oligonucleotides each comprise a nucleic acid sequence comprising any one of SEQ ID NOs: 45-47 and 73-75. In some embodiments, the one or more RNAi oligonucleotides each comprise a spacer that consists of any one of SEQ ID NOs: 45-47 and 73-75.

In some embodiments, the expression control component comprises one or more nucleic acid sequences encoding for one or more RNAi oligonucleotides each comprising a nucleic acid sequence sufficiently complementary to mRNA encoding Rep68 to direct RNAi-mediated degradation of Rep68. In some embodiments, the one or more RNAi oligonucleotides each comprise a nucleic acid sequence sufficiently complementary to mRNA that encodes SEQ ID NO: 9 to direct RNAi-mediated degradation of the mRNA. In some embodiments, the one or more RNAi oligonucleotides sufficiently complementary to mRNA of Rep68 to direct RNAi-mediated degradation of Rep68 each comprise a nucleic acid sequence comprising at least 80% (e.g., at least 80%, at least 85%, at least 90%, at least 95%, or at least 99%) identity to any one of SEQ ID NOs: 45-47 and 73-75. In some embodiments, the one or more RNAi oligonucleotides each comprise a nucleic acid sequence comprising any one of SEQ ID NOs: 45-47 and 73-75. In some embodiments, the one or more RNAi oligonucleotides each comprise a spacer that consists of any one of SEQ ID NOs: 45-47 and 73-75.

In some embodiments, the expression control component comprises one or more nucleic acid sequences encoding for one or more RNAi oligonucleotides each comprising a nucleic acid sequence sufficiently complementary to mRNA encoding Rep to direct RNAi-mediated degradation of Rep. In some embodiments, the one or more RNAi oligonucleotides each comprise a nucleic acid sequence sufficiently complementary to mRNA encoded by SEQ ID NO: 20 to direct RNAi-mediated degradation of the mRNA. In some embodiments, the one or more RNAi oligonucleotides sufficiently complementary to mRNA of Rep to direct RNAi-mediated degradation of Rep each comprise a nucleic acid sequence comprising at least 80% (e.g., at least 80%, at least 85%, at least 90%, at least 95%, or at least 99%) identity to any one of SEQ ID NOs: 45-47 and 73-75. In some embodiments, the one or more RNAi oligonucleotides each comprise a nucleic acid sequence comprising any one of SEQ ID NOs: 45-47 and 73-75. In some embodiments, the one or more RNAi oligonucleotides each comprise a spacer that consists of any one of SEQ ID NOs: 45-47 and 73-75.

In some embodiments, the expression control component comprises one or more nucleic acid sequences encoding for one or more RNAi oligonucleotides each comprising a nucleic acid sequence sufficiently complementary to mRNA encoding E2A to direct RNAi-mediated degradation of E2A. In some embodiments, the one or more RNAi oligonucleotides each comprise a nucleic acid sequence sufficiently complementary to mRNA that encodes SEQ ID NO: 10 to direct RNAi-mediated degradation of the mRNA. In some embodiments, the one or more RNAi oligonucleotides sufficiently complementary to mRNA of E2A to direct RNAi-mediated degradation of E2A each comprise a nucleic acid sequence comprising at least 80% (e.g., at least 80%, at least 85%, at least 90%, at least 95%, or at least 99%) identity to SEQ ID NO: 29 or SEQ ID NOs: 39-41 and 67-79. In some embodiments, the one or more RNAi oligonucleotides each comprise a nucleic acid sequence comprising SEQ ID NOs: 39-41 and 67-79. In some embodiments, the one or more RNAi oligonucleotides each comprise a spacer that consists of SEQ ID NOs: 39-41 and 67-79.

In some embodiments, the expression control component comprises one or more nucleic acid sequences encoding for one or more RNAi oligonucleotides each comprising a nucleic acid sequence sufficiently complementary to mRNA encoding E4ORF6 to direct RNAi-mediated degradation of E4ORF6. In some embodiments, the one or more RNAi oligonucleotides each comprise a nucleic acid sequence sufficiently complementary to mRNA that encodes SEQ ID NO: 11 to direct RNAi-mediated degradation of the mRNA. In some embodiments, the one or more RNAi oligonucleotides sufficiently complementary to mRNA of E4ORF6 to direct RNAi-mediated degradation of E4ORF6 each comprise a nucleic acid sequence comprising at least 80% (e.g., at least 80%, at least 85%, at least 90%, at least 95%, or at least 99%) identity to SEQ ID NOs: 42-44 and 70-72. In some embodiments, the one or more RNAi oligonucleotides each comprise a nucleic acid sequence comprising SEQ ID NOs: 42-44 and 70-72. In some embodiments, the one or more RNAi oligonucleotides each comprise a spacer that consists of SEQ ID NOs: 42-44 and 70-72.

In some embodiments, the expression control component comprises one or more nucleic acid sequences encoding for one or more RNAi oligonucleotides each comprising a nucleic acid sequence sufficiently complementary to mRNA encoding VP1 to direct RNAi-mediated degradation of VP1. In some embodiments, the one or more RNAi oligonucleotides each comprise a nucleic acid sequence sufficiently complementary to mRNA that encodes SEQ ID NO: 14 to direct RNAi-mediated degradation of the mRNA. In some embodiments, the one or more RNAi oligonucleotides sufficiently complementary to mRNA of VP1 to direct RNAi-mediated degradation of VP1 each comprise a nucleic acid sequence comprising at least 80% (e.g., at least 80%, at least 85%, at least 90%, at least 95%, or at least 99%) identity to SEQ ID NOs: 48-50 and 76-78. In some embodiments, the one or more RNAi oligonucleotides each comprise a nucleic acid sequence comprising SEQ ID NOs: 48-50 and 76-78. In some embodiments, the one or more RNAi oligonucleotides each comprise a spacer that consists of SEQ ID NOs: 48-50 and 76-78.

In some embodiments, the expression control component comprises one or more nucleic acid sequences encoding for one or more RNAi oligonucleotides each comprising a nucleic acid sequence sufficiently complementary to mRNA encoding VP2 to direct RNAi-mediated degradation of VP2. In some embodiments, the one or more RNAi oligonucleotides each comprise a nucleic acid sequence sufficiently complementary to mRNA that encodes SEQ ID NO: 15 to direct RNAi-mediated degradation of the mRNA. In some embodiments, the one or more RNAi oligonucleotides sufficiently complementary to mRNA of VP2 to direct RNAi-mediated degradation of VP2 each comprise a nucleic acid sequence comprising at least 80% (e.g., at least 80%, at least 85%, at least 90%, at least 95%, or at least 99%) identity to SEQ ID NOs: 48-50 and 76-78. In some embodiments, the one or more RNAi oligonucleotides each comprise a nucleic acid sequence comprising SEQ ID NOs: 48-50 and 76-78. In some embodiments, the one or more RNAi oligonucleotides each comprise a spacer that consists of SEQ ID NOs: 48-50 and 76-78.

In some embodiments, the expression control component comprises one or more nucleic acid sequences encoding for one or more RNAi oligonucleotides each comprising a nucleic acid sequence sufficiently complementary to mRNA encoding VP3 to direct RNAi-mediated degradation of VP3. In some embodiments, the one or more RNAi oligonucleotides each comprise a nucleic acid sequence sufficiently complementary to mRNA that encodes SEQ ID NO: 16 to direct RNAi-mediated degradation of the mRNA. In some embodiments, the one or more RNAi oligonucleotides sufficiently complementary to mRNA of VP3 to direct RNAi-mediated degradation of VP3 each comprise a nucleic acid sequence comprising at least 80% (e.g., at least 80%, at least 85%, at least 90%, at least 95%, or at least 99%) identity to SEQ ID NOs: 48-50 and 76-78. In some embodiments, the one or more RNAi oligonucleotides each comprise a nucleic acid sequence comprising SEQ ID NOs: 48-50 and 76-78. In some embodiments, the one or more RNAi oligonucleotides each comprise a spacer that consists of SEQ ID NOs: 48-50 and 76-78.

In some embodiments, the expression control component comprises one or more nucleic acid sequences encoding for one or more RNAi oligonucleotides each comprising a nucleic acid sequence sufficiently complementary to mRNA encoding AAP to direct RNAi-mediated degradation of AAP. In some embodiments, the one or more RNAi oligonucleotides each comprise a nucleic acid sequence sufficiently complementary to mRNA that encodes SEQ ID NO: 17 to direct RNAi-mediated degradation of the mRNA. In some embodiments, the one or more RNAi oligonucleotides sufficiently complementary to mRNA of AAP to direct RNAi-mediated degradation of AAP each comprise a nucleic acid sequence comprising at least 80% (e.g., at least 80%, at least 85%, at least 90%, at least 95%, or at least 99%) identity to SEQ ID NO: 48 and 76. In some embodiments, the one or more RNAi oligonucleotides each comprise a nucleic acid sequence comprising SEQ ID NOs: 48 and 76. In some embodiments, the one or more RNAi oligonucleotides each comprise a spacer that consists of SEQ ID NOs: 48 and 76.

In some embodiments, the expression control component comprises one or more nucleic acid sequences encoding for one or more RNAi oligonucleotides each comprising a nucleic acid sequence sufficiently complementary to RNA encoding VARNA to direct RNAi-mediated degradation of VARNA. In some embodiments, the one or more RNAi oligonucleotides each comprise a nucleic acid sequence sufficiently complementary to mRNA encoded by SEQ ID NO: 13 to direct RNAi-mediated degradation of the mRNA.

In some embodiments, the expression control component comprises one or more nucleic acid sequences encoding for one or more RNAi oligonucleotides each comprising a nucleic acid sequence sufficiently complementary to mRNA encoding MAAP to direct RNAi-mediated degradation of MAAP. In some embodiments, the one or more RNAi oligonucleotides each comprise a nucleic acid sequence sufficiently complementary to mRNA that encodes SEQ ID NO: 80 to direct RNAi-mediated degradation of the mRNA.

### IX. Exemplary Embodiments of Engineered Cells Comprising RNAi Oligonucleotides

In some aspects, the AAV production system further comprises an engineered cell for AAV production as described above. In some embodiments, the engineered cell comprises the one or more nucleic acid molecules collectively comprising: (a) an AAV production component as described above and (b) an expression control component comprises RNAi oligonucleotides as described above. In some embodiments, the AAV production component and the expression control component are stably integrated into the genome of the engineered cell. In some embodiments, the AAV production component and the expression control component (or any combination of components of the AAV production system) are transfected into the engineered cell as a vector or a plasmid.

In some embodiments, the engineered cell further comprises one or more nucleic acid molecules encoding a transcriptional activator as described above. In some embodiments, the one or more nucleic acid molecules encoding a transcriptional activator as described above are stably integrated.

In some embodiments, each of the nucleic acid molecules of the AAV production system comprises a selection marker. In some embodiments, each nucleic acid molecule of the AAV production system comprises a nucleic acid sequence of a distinct selection marker.

### A. The First Stably Integrated Nucleic Acid Molecule

In some embodiments, the engineered cell for AAV production comprising one or more nucleic acid molecules comprises a first stably integrated nucleic acid molecule. In some embodiments, the first stably integrated nucleic acid molecule comprises a nucleic acid sequence encoding for each of one or more RNAi oligonucleotides as described herein. In some embodiments, the one or more RNAi oligonucleotides are operably linked to a promoter (constitutive or inducible) as described herein. In some embodiments, the one or more RNAi oligonucleotides are each operably linked to a CMV promoter.

In some embodiments, the one or more RNAi oligonucleotides each comprise a nucleic acid sequence sufficiently complementary to any one of the nucleic acid sequences encoding Rep52 or Rep40 to directed RNAi-mediated degradation of the mRNA of Rep52 or Rep40 (as described herein).

In some embodiments, the one or more RNAi oligonucleotides each comprise a nucleic acid sequence sufficiently complementary to any one of the nucleic acid sequences encoding Rep78 or Rep68, to directed RNAi-mediated degradation of the mRNA of Rep78 or Rep68 (as described herein).

In some embodiments, the one or more RNAi oligonucleotides each comprise a nucleic acid sequence sufficiently complementary to any one of the nucleic acid sequences encoding E2A, to directed RNAi-mediated degradation of the mRNA of E2A (as described herein).

In some embodiments, the one or more RNAi oligonucleotides each comprise a nucleic acid sequence sufficiently complementary to any one of the nucleic acid sequences encoding E4Orf6 to directed RNAi-mediated degradation of the mRNA of E4Orf6 (as described herein).

In some embodiments, the one or more RNAi oligonucleotides each comprise a nucleic acid sequence sufficiently complementary to any one of the nucleic acid sequences encoding Rep52 or Rep40, Rep78 or Rep68, E2A, and E4Orf6 to directed RNAi-mediated degradation of the mRNA of Rep52 or Rep40, Rep78 or Rep68, E2A, and E4Orf6 (as described herein).

In some embodiments, the first stably integrated nucleic acid molecule comprises one or more nucleic acid sequences encoding each of one or more RNAi oligonucleotides that comprises a nucleic sequence encoding for any one of SEQ ID NOs: 39-50 and 67-78 operably linked to a promoter (as described herein).

In some embodiments, the first stably integrated nucleic acid molecule comprises a selection cassette as described herein. In some embodiments, the first stably integrated nucleic acid molecule further comprises a nucleic acid sequence encoding for Neo-TagBFP.

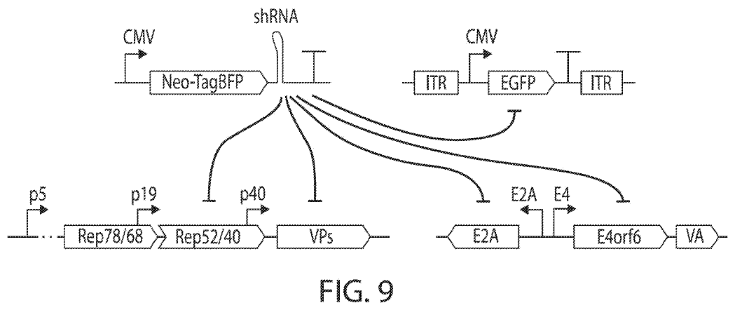

In some embodiments, the first stably integrated nucleic acid molecule as described above has the same structure as is depicted in the schematic of FIG. 9 comprising RNAi oligonucleotides.

In some embodiments, the first stably integrated nucleic acid molecule further comprises two IR/DR sequences that are capable of binding a Sleeping Beauty transposase. In some embodiments, the IR/DR sequences are SB100X ID/DR sequences. In some embodiments, it is to be understood that the any combination of the components of the first stably integrated nucleic acid molecule as described above may be encoded on a plasmid or vector and delivered to the engineered cell without stable integration.

### B. The Second Stably Integrated Nucleic Acid Molecule

In some embodiments, the engineered cell for AAV production comprising one or more stably integrated nucleic acid molecules comprises a first stably integrated nucleic acid molecule as described above, further comprises a second stably integrated nucleic acid molecule. In some embodiments, the second stably integrated nucleic acid molecule comprises one or more nucleic acid sequences collectively encoding all or any portion of the AAV production component (as described herein).

In some embodiments, the second stably integrated nucleic acid molecule comprises one or more nucleic acid sequence encoding for Rep52 or Rep40 (as described herein) operably linked to a promoter (as described herein).

In some embodiments, the second stably integrated nucleic acid molecule comprises one or more nucleic acid sequence encoding for Rep78 or Rep68 (as described herein) operably linked to a promoter (as described herein).

In some embodiments, the second stably integrated nucleic acid molecule comprises one or more nucleic acid sequence encoding for VP1, VP2, and VP3 (as described herein) operably linked to a promoter (as described herein).

In some embodiments, the second stably integrated nucleic acid molecule comprises a nucleic acid sequence encoding for AAP (as described herein) operably linked to a promoter (as described herein).

In some embodiments, the second stably integrated nucleic acid molecule comprises one or more nucleic acid sequence encoding for Rep52 or Rep40; Rep78 or Rep68; VP1, VP2, and VP3; and AAP (as described herein) each operably linked to a promoter (as described herein).

In some embodiments, the second stably integrated nucleic acid molecule comprises a selection cassette as described herein.

In some embodiments, the second stably integrated nucleic acid molecule further comprises two IR/DR sequences that are capable of binding a Sleeping Beauty transposase. In some embodiments, the IR/DR sequences are SB100X ID/DR sequences.

In some embodiments, the second stably integrated nucleic acid molecule as described above has the same structure as is depicted in the schematic of FIG. 9 comprising Rep52 or Rep40; Rep78 or Rep68; VP1, VP2, and VP3; and AAP.

### C. The Third Stably Integrated Nucleic Acid Molecule

In some embodiments, the engineered cell for AAV production comprising one or more stably integrated nucleic acid molecules comprises a first stably integrated nucleic acid molecule as described above, a second stably integrated nucleic acid molecule as described above, and comprises a third stably integrated nucleic acid molecule. In some embodiments, the third stably integrated nucleic acid molecule comprises one or more nucleic acid sequences collectively encoding all or any portion of the AAV production component (as described herein).

In some embodiments, the third stably integrated nucleic acid molecule comprises a nucleic acid sequence encoding for E2A (as described herein) operably linked to a promoter (as described herein).

In some embodiments, the third stably integrated nucleic acid molecule comprises a nucleic acid sequence encoding for E4Orf6 (as described herein) operably linked to a promoter (as described herein).

In some embodiments, the third stably integrated nucleic acid molecule comprises a nucleic acid sequence encoding for VARNA (as described herein) operably linked to a promoter (as described herein).

In some embodiments, the third stably integrated nucleic acid molecule comprises one or more nucleic acid sequence encoding for E2A, E4Orf6, and VARNA (as described herein) each operably linked to a promoter (as described herein).

In some embodiments, the third stably integrated nucleic acid molecule comprises a selection cassette as described herein.

In some embodiments, the third stably integrated nucleic acid molecule further comprises two IR/DR sequences that are capable of binding a Sleeping Beauty transposase. In some embodiments, the IR/DR sequences are SB100X ID/DR sequences.

In some embodiments, the third stably integrated nucleic acid molecule as described above has the same structure as is depicted in the schematic of FIG. 9 comprising E2A, E4Orf6, and VARNA.

### D. The Fourth Stably Integrated Nucleic Acid Molecule

In some embodiments, the engineered cell for AAV production comprising one or more stably integrated nucleic acid molecules comprises a first stably integrated nucleic acid molecule as described above, a second stably integrated nucleic acid molecule as described above, a third stably integrated nucleic acid molecule as described above and comprises a fourth stably integrated nucleic acid molecule. In some embodiments, the fourth stably integrated nucleic acid molecule comprises a nucleic acid sequence encoding transcriptional activator as described above. In some embodiments, the fourth stably integrated nucleic acid molecule further comprises two IR/DR sequences that are capable of binding a Sleeping Beauty transposase. In some embodiments, the IR/DR sequences are SB100X ID/DR sequences.

### E. The Fifth Stably Integrated Nucleic Acid Molecule

In some embodiments, the engineered cell for AAV production comprising one or more stably integrated nucleic acid molecules comprises a first stably integrated nucleic acid molecule as described above, a second stably integrated nucleic acid molecule as described above, a third stably integrated nucleic acid molecule as described above, a fourth stably integrated nucleic acid molecule as described above and comprises a fifth stably integrated nucleic acid molecule. In some embodiments, the fifth stably integrated nucleic acid molecule comprises a nucleic acid sequence encoding each of a selection cassette operably linked to a promoter as described above. In some embodiments, the fifth stably integrated nucleic acid molecule comprises a payload operably linked to a promoter. In some embodiments, the payload is a nucleic acid sequence encoding a fluorescent protein marker (as described herein), such as EGFP. In some embodiments, the fifth stably integrated nucleic acid molecule further comprises two inverted terminal repeat (ITR) sequences. In some embodiments, the fifth stably integrated nucleic acid molecule further comprises two IR/DR sequences that are capable of binding a Sleeping Beauty transposase. In some embodiments, the IR/DR sequences are SB100X ID/DR sequences.

### X. Methods of Using Engineered Cells for AAV Production Comprising RNAi Oligonucleotides.

In some aspects, the present disclosure provides methods for producing AAV using an AAV production system comprising one or more nucleic acid molecules collectively comprising: (a) an AAV production component and (b) an expression control component comprising RNAi oligonucleotides as described herein. In some embodiments, the method of AAV production comprises transfecting or stably integrating into an engineered cell any combination of the one or more nucleic acid molecules collectively comprising the AAV production component and the expression control component comprising RNAi oligonucleotides as described herein. In some embodiments, the method of AAV production further comprises transfecting a nucleic acid molecule comprising a payload for AAV delivery (e.g. a therapeutic DNA sequence) as described above. In some embodiments, the engineered cell used in the method of AAV production is selected from any one of the engineered cells for AAV production comprising one or more RNAi oligonucleotides described herein. In some embodiments, the method comprises growing the engineered cell to a confluency that is optimal for AAV production. An optimal confluency may be dependent, for example, on the type of cell the engineered cell is derived from. The skilled person will know or be able to determine the optimal confluency for AAV production. In some embodiments, the method comprises culturing the engineered cell with a small molecule inducer capable of inducing expression of the one or more RNAi oligonucleotides as described above. In some embodiments, the method comprises harvesting the AAV produced from the culture of engineered cells using methods that are well known to those of skill in the art.

### XI. Kits

In some aspects, the disclosure relates to kits comprising a AAV production systems described herein.

In some embodiments, a kit comprises one or more nucleic acid molecules collectively comprising an AAV production system.

In some embodiments, a kit comprises an engineered cell described in Parts III, VI and IX.

In some embodiments, a kit comprises a nucleic acid sequence comprising, from 5′ to 3′: (i) a nucleic acid sequence of a 5′ inverted tandem repeat; (ii) a multiple cloning site; and (iii) a nucleic acid sequence of a 3′ inverted tandem repeat. In some embodiments, the nucleic acid sequence is a plasmid or a vector.

The central nucleic acid of a transfer nucleic acid molecule may comprise a nucleic acid sequence of a multiple cloning site. Exemplary multiple cloning sites are known to those having ordinary skill in the art. A multiple cloning site can be used for cloning a payload molecule (or gene of interest)—or an expression cassette encoding a payload molecule—into the transfer nucleic acid molecule prior to the generation of viral vectors in a host cell.

In some embodiments, the kit further comprises a small molecule inducer corresponding to a chemically inducible promoter of the AAV production system. In some embodiments, a small molecule inducer is doxycycline, vanillate, phloretin, rapamycin, abscisic acid, gibberellic acid acetoxymethyl ester, and cumate.

In some embodiments, the kit further comprises a molecule capable of binding and stabilizing the degradation domain of the expression control system. In some embodiments, the molecule is trimethoprim, Shield1, auxin, antizyme, rapamycin, or a rapamycin analog.

In some embodiments, a kit comprises an engineered cell, wherein the engineered cell comprises the stably integrated nucleic acid molecules of sections III, VI and IX.

In some embodiments, a kit comprises a nucleic acid molecule comprising a nucleic acid sequence of a transcriptional activator operably linked to a nucleic acid sequence of a promoter, wherein the transcriptional activator, when expressed in the presence of the small molecule inducer, binds to a chemically inducible promoter of the AAV production system, optionally wherein an engineered cell comprises the nucleic acid molecule comprising the nucleic acid sequence of the transcriptional activator. In some embodiments, the transcriptional activator is selected from the group consisting of TetOn-3G, TetOn-V16, TetOff-Advanced, VanR-VP16, TtgR-VP16, PhIF-VP16, and the cumate-responsive transactivators cTA and rcTA.

In some embodiments, the kits may further comprise instructions for use of the cells.

### EXAMPLES

Viral vectors are a promising gene delivery modality for cell and gene therapy. The production of viral vectors normally entails transient transfection of plasmids into cell culture. However, stable integration of genes necessary to produce therapeutic viral vectors into the genome offers several advantages compared to traditional production via transient transfection. Since cells amplify the viral genes during their own cell division, large quantities of DNA and transfection reagent no longer need to be procured for the transfection process, reducing costs. Also since the DNA is already within the nucleus, viral titers may be higher and more consistent due to minimal numbers of ‘untransfected’ cells and reduced variation associated with transfection steps. The simpler production process also saves scientist time.

However, several genes required for adeno-associated viral (AAV) vector production have been demonstrated by others to be cytostatic or cytotoxic, namely Rep, E2A and E4. The cytotoxic and cytostatic nature of these proteins has hampered the development of stable AAV producer cell lines in the widely used HEK293 cell line, since the native expression of adenovirus E1 genes in HEK293 cells upregulates expression of these toxic genes. Cells stably transfected with these genes fail to survive selection steps or have silenced expression, resulting in an inability to produce relevant quantities of AAV.

The following designs introduce inducible control of cytostatic or cytotoxic genes at various stages (genetic, transcriptional, post-transcriptional, post-translational). Each of the described constructs can be integrated into the genome using random integration, targeted integration, or transposon-mediated integration.

### Example 1: AAV Expression Control with a Degradation Domain

### Description of Approach and Genetic Schematic:

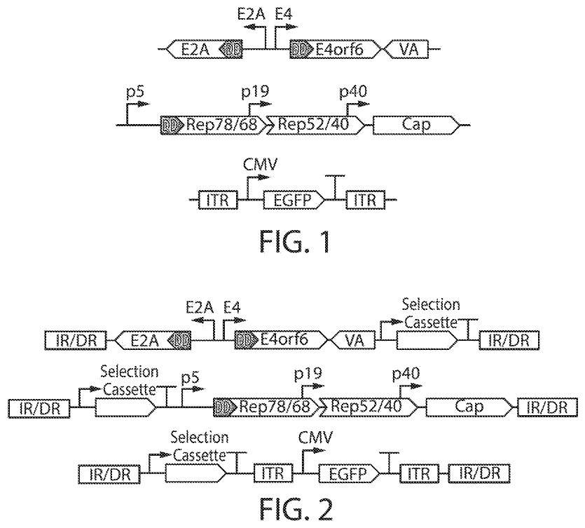

To reduce expression of cytostatic or cytotoxic genes when generating a stable cell line, degradation domain (DD) fusions were employed to destabilize the proteins (FIG. 1-2). Some degradation domains are small molecule-binding proteins that have been mutated to be destabilized and degraded by the cell. As a result, proteins fused to these degradation domain will also be degraded. It was hypothesized that with degradation domains fused to Rep, E2A, and E4, the toxic proteins would be mostly degraded, resulting in reduced AAV titers, improved cell health, and therefore improved ability to make stable AAV producer cells. When production of AAV is desired, protein expression can be rescued by addition of a small molecule (Shield1 or trimethoprim), which binds the degradation domain in a way that reduces the destabilizing effects.

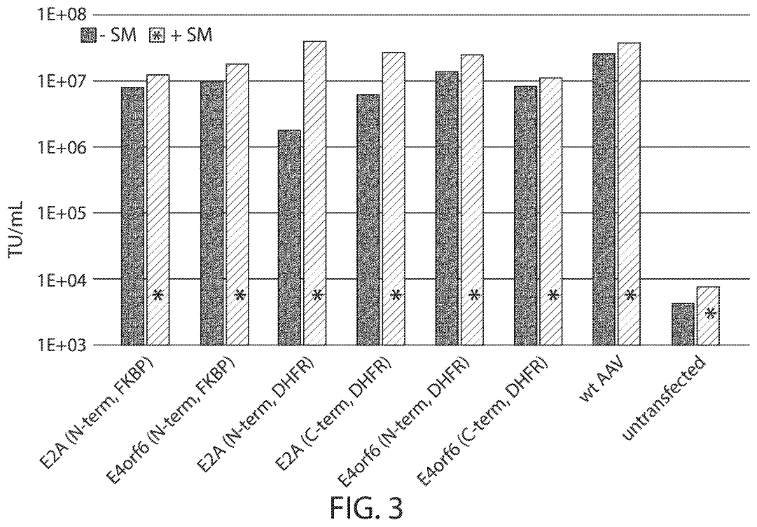

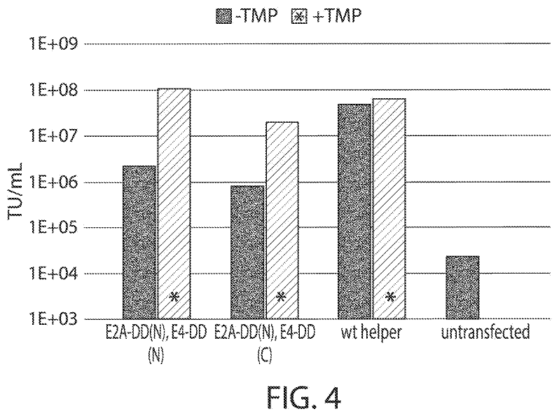

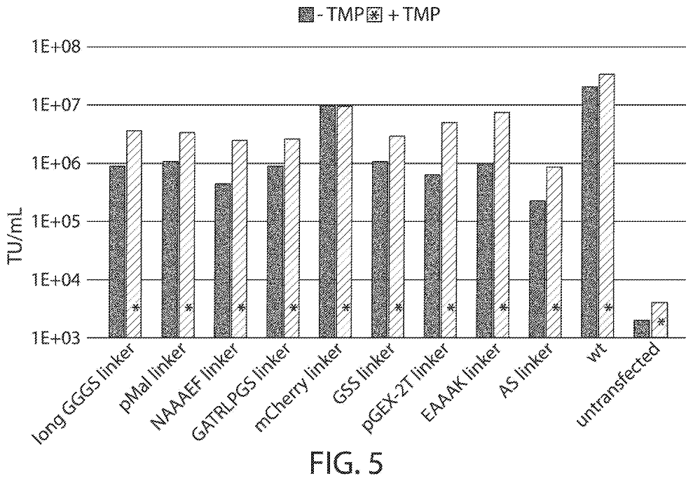

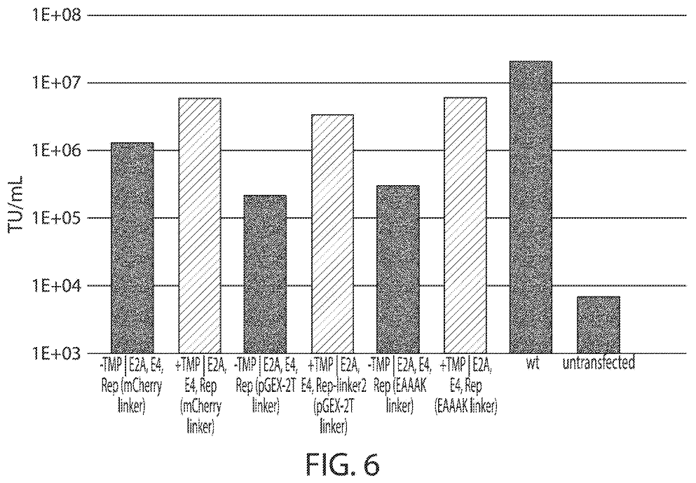

In the tests, direct fusions of degradation domains to the E2A and E4 helper genes with a short linker (e.g., a GSG linker) are well tolerated upon small molecule treatment (FIGS. 3-4, and 6). For Rep, specific linker sequences appeared to have effects on AAV titers so several variants were designed and tested (FIG. 5). The overall genetic design is based on the current standard plasmid system, consisting of a pRepCap plasmid containing the AAV Rep and Cap sequences but lacking ITRs, a pHelper system containing the adenovirus E2A, E4, and VA genes, and an AAV transfer plasmid containing expression of a gene of interest (EGFP in the tests) flanked by AAV ITRs. These standard plasmids were modified to append degradation domains to the relevant coding sequences. Initial tests to generate preliminary data were performed with plasmids lacking selection cassettes and Sleeping Beauty IR/DRs, which are relevant for generating stable cells but not necessary for testing induction of AAV.

There are several other aspects of the inducible AAV platform that may be beneficial for large scale production of therapeutic gene therapy vectors. Viral genes are split across multiple plasmids so that they are integrated in separate parts of the genome, resulting in increased safety due to decreased ability to form competent virus.

### Preliminary Data and Experiment Description:

Adherent HEK293FT cells were co-transfected with EGFP-expressing transfer plasmid, modified pRepCap, and modified pHelper (FIG. 1). Control samples containing ‘wild type’ AAV2 pRepCap and pHelper plasmids or completely untransfected were also prepared. Small molecules Shield1 or trimethoprim (TMP) were added to cells at the time of transfection at 1 uM or 10 uM concentration respectively. 48-72 hours after transfection, AAV was harvested by three freeze thaw cycles in a dry ice isopropanol bath. Virus stock was serially diluted 1-, 10- and 100-fold and 10 uL of resulting viral stocks was transduced by addition to 5e4 HEK293FT cells plated in a 96-well plate. 48-72 hours after transduction, transduced cells were harvested and percentage of EGFP positive cells was determined by flow cytometry and used to calculate transducing units per mL (TU/mL).

### Example 2: AAV Gene Product Expression Control with Cas13

### Description of Approach and Genetic Schematic:

Cas13-mediated degradation of the Rep, E2A, and E4orf6 mRNA is another method to reduce cytotoxic and cytostatic effects when generating a stable producer cell line. Cas13 is a RNA-targeting CRISPR enzyme that complexes with a guide RNA (crRNA) complementary to the target RNA to then bind and cleave the target RNA. It was hypothesized that expression of Cas13 and crRNAs targeting the mRNAs of toxic genes required for AAV production would lead to reduced titers and the ability to generate stable AAV producer cells. Cas13 expression can be regulated by an inducible promoter (e.g. TRE3G) and the half-life of Cas13 protein regulated by a degradation domain (e.g. the c-terminal region of ornithine decarboxylase or mutants thereof) such that when production of AAV is desired the addition of a molecule (e.g., a small molecule or protein) will halt expression of Cas13 leading to up regulation of Rep, E2A, and E4orf6.

Production of AAV by transient transfection of the standard plasmid system along with plasmids for constitutive expression of RfxCas13d and a single crRNA targeting Rep, E2A, or E4orf6 yielded reduced titers. The reduction in titer does exhibit some dependence on the sequence of the crRNA.

### Preliminary Data and Experiment Description:

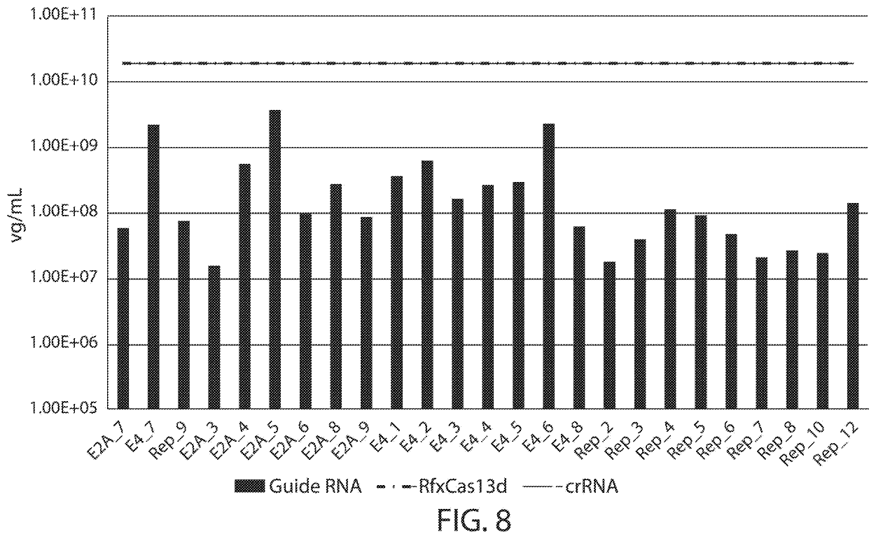

Adherent HEK293FT cells were co-transfected with EGFP-expressing transfer plasmid, pRepCap, pHelper, RfxCas13d, and crRNA plasmids (FIG. 7). crRNA plasmids expressed a single guide RNA targeting Rep, E2A, or E4orf6 transcripts. Samples lacking either RfxCas13d (−RfxCas13d) or crRNA (−rRNA) were co-transfected with an inert plasmid to maintain constant DNA amount in all samples. 72 hours after transfection, AAV was harvested by four freeze thaw cycles in a dry ice isopropanol bath. Virus stock was treated with DNase and Proteinase K, then serially diluted 1000- and 10,000-fold and titered by droplet digital PCR (ddPCR) with primer and probe sequences targeting eGFP. The ddPCR concentration values were used to calculate viral genomes per mL (vg/mL) (FIG. 8).

Inducible, half life-regulated RfxCas13d, a pool of crRNA, and AAV production genes are stably integrated into a suspension HEK293 cell line to yield a stable AAV Producer cell line.

### Example 3: AAV Gene Product Expression Control with shRNA

### Description of Approach and Genetic Schematic:

Small hairpin RNAs (shRNAs) are employed to downregulate AAV-related genes in order to fine-tune expression (FIG. 9). This may be used in combination with other inducible AAV systems to further downregulate select genes that may have leaky expression without shRNAs. The shRNA backbone used in these preliminary studies is based on miR-E and placed in the 3′ UTR immediately downstream of a PolII-driven gene (Neo-TagBFP) and flanked by splicing donor and acceptor sequences along with a polypyrimidine tract. Alternative backbones besides miR-E may be used. RNA hairpins are transcribed, spliced out, and processed to generate shRNAs which are then used to downregulated target genes through RNAi. shRNAs can also target the gene of interest (GOI) to reduce toxic effects which would enhance titers and stability of the producer cell line. Clusters of shRNAs may also be generated from a single PolII promoter, rather than each generated from a separate promoter, to reduce genetic size and redundancy and increase reproducibility.

### Preliminary Data and Experiment Description:

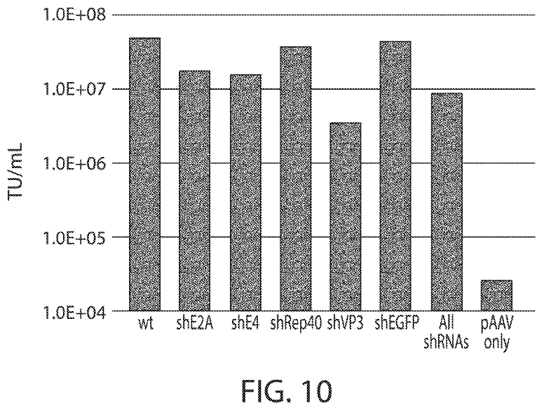

AAV pHelper, AAV pRepCap, and transfer plasmids were co-transfected along with the shRNA expression plasmid into HEK293FT cells. For each target, 2-3 different shRNA plasmids were pooled together prior to testing. An additional sample used all shRNAs pooled together. Control samples containing ‘wild type’ AAV2 pRepCap and pHelper plasmids but without shRNA, or completely untransfected were also prepared. 72 hours after transfection, AAV was harvested by three freeze thaw cycles in a dry ice isopropanol bath. Virus stock was serially diluted 1-, 10- and 100-fold and 10 uL of resulting viral stocks was transduced by addition to 5e4 HEK293FT cells plated in a 96-well plate. 48 hours after transduction, transduced cells were harvested and percentage of EGFP positive cells was determined by flow cytometry and used to calculate transducing units per mL (TU/mL) (FIG. 10).

TABLE 1
Nucleic Acid and Polypeptide Sequences

| SEQ ID | | |
|---|---|---|
| NO: | Description. | Sequence |
| 1 | pTREtight | ctcgagtttactccctatcagtgatagagaacgtatgtcgagtttactccctatcagtgatagagaac |
| | | gatgtcgagtttactccctatcagtgatagagaacgtatgtcgagtttactccctatcagtgatagag |
| | | aacgtatgtcgagtttactccctatcagtgatagagaacgtatgtcgagtttatccctatcagtgata |
| | | gagaacgtatgtcgagtttactccctatcagtgatagagaacgtatgtcgaggtaggcgtgtacgg |
| | | tgggaggcctatataagcagagctcgtttagtgaaccgtcagatcgcctggagaattcgagctcg |
| | | gtacccgggga |
| 2 | pTRE3G | gtttactccctatcagtgatagagaacgtatgaagagtttactccctatcagtgatagagaacgtat |
| | | gcagactttactccctatcagtgatagagaacgtataaggagtttactccctatcagtgatagagaa |
| | | cgtatgaccagtttactccctatcagtgatagagaacgtatctacagtttactccctatcagtgatag |
| | | agaacgtatatccagtttactccctatcagtgatagagaacgtataagctttaggcgtgtacggtgg |
| | | gcgcctataaaagcagagctcgtttagtgaaccgtcagatcgcctggagcaattccacaacacttt |
| | | tgtcttataccaactttccgtaccacttcctaccctcgtaaagtcgacaccggggcccagatctatc |
| | | gatcggccggataacgccacc |
| 3 | bi-TRE3G | gaattctccaggcgatctgacggttcactaaacgagctctgcttatataggcctcccaccgtacac |
| | | gccacctcgacatactcgagtttactccctatcagtgatagagaacgtatgaagagtttactcccta |
| | | tcagtgatagagaacgtatgcagactttactccctatcagtgatagagaacgtataaggagtttact |
| | | ccctatcagtgatagagaacgtatgaccagtttactccctatcagtgatagagaacgtatctacagt |
| | | ttactccctatcagtgatagagaacgtatatccagtttactccctatcagtgatagagaacgtataag |
| | | ctttaggcgtgtacggtgggcgcctataaaagcagagctcgtttagtgaaccgtcagatcgcctg |
| | | gagcaattccacaacacttttgtcttataccaactttccgtaccacttcctaccctcgtaaagtcgac |
| | | accggggcccagatctccgcggggatcc |
| 4 | IRES | cccctctccctcccccccccctaacgttactggccgaagccgcttggaataaggccggtgtgcgttt |
| | | gtctatatgttattttccaccatattgccgtcttttggcaatgtgagggcccggaaacctggccctg |
| | | tcttcttgacgagcattcctaggggtctttcccctctcgccaaaggaatgcaaggtctgttgaatgtc |
| | | gtgaaggaagcagttcctctggaagcttcttgaagacaaacaacgtctgtagcgaccctttgcag |
| | | gcagcggaaccccccacctggcgacaggtgcctctgcggccaaaagccacgtgtataagatac |
| | | acctgcaaaggcggcacaaccccagtgccacgttgtgagttggatagttgtggaaagagtcaaa |
| | | tggctctcctcaagcgtattcaacaaggggctgaaggatgcccagaaggtaccccattgtatggg |
| | | atctgatctggggcctcggtgcacatgctttacatgtgtttagtcgaggttaaaaaaacgtctaggc |
| | | cccccgaaccacggggacgtggttttcctttgaaaaacacgatgataatatg |
| 5 | attenuated | cccccccccctaacgttactggccgaagccgcttggaataaggccggtgtgcgtttgtctatatgt |
| | IRES | tattttccaccatattgccgtcttttggcaatgtgagggcccggaaacctggccctgtcttcttgacg |
| | | agcattcctaggggtctttcccctctcgccaaaggaatgcaaggtctgttgaatgtcgtgaaggaa |
| | | gcagttcctctggaagcttcttgaagacaaacaacgtctgtagcgaccctttgcaggcagcggaa |
| | | ccccccacctggcgacaggtgcctctgcggccaaaagccacgtgtataagatacacctgcaaa |
| | | ggcggcacaaccccagtgccacgttgtgagttggatagttgtggaaagagtcaaatggctctcct |
| | | caagcgtattcaacaaggggctgaaggatgcccagaaggtaccccattgtatgggatctgatctg |
| | | gggcctcggtgcacatgctttacatgtgtttagtcgaggttaaaaaaacgtctaggccccccgaac |
| | | cacggggacgtggttttcctttgaaaaacacgatgataatagttatc |
| 6 | Rep52 (wt) | MELVGWLVDKGITSEKQWIQEDQASYISFNAASNSRSQIKAA |
| | | LDNAGKIMSLTKTAPDYLVGQQPVEDISSNRIYKILELNGYDP |
| | | QYAASVFLGWATKKFGKRNTIWLFGPATTGKTNIAEAIAHT |
| | | VPFYGCVNWTNENFPFNDCVDKMVIWWEEGKMTAKVVES |
| | | AKAILGGSKVRVDQKCKSSAQIDPTPVIVTSNTNMCAVIDGN |
| | | STTFEHQQPLQDRMFKFELTRRLDHDFGKVTKQEVKDFFRW |
| | | AKDHVVEVEHEFYVKKGGAKKRPAPSDADISEPKRVRESVA |
| | | QPSTSDAEASINYADRYQNKCSRHVGMNLMLFPCRQCERMN |
| | | QNSNICFTHGQKDCLECFPVSESQPVSVVKKAYQKLCYIHHI |
| | | MGKVPDACTACDLVNVDLDDCIFEQ |
| 7 | Rep40 (wt) | MELVGWLVDKGITSEKQWIQEDQASYISFNAASNSRSQIKAA |
| | | LDNAGKIMSLTKTAPDYLVGQQPVEDISSNRIYKILELNGYDP |
| | | QYAASVFLGWATKKFGKRNTIWLFGPATTGKTNIAEAIAHT |
| | | VPFYGCVNWTNENFPFNDCVDKMVIWWEEGKMTAKVVES |
| | | AKAILGGSKVRVDQKCKSSAQIDPTPVIVTSNTNMCAVIDGN |
| | | STTFEHQQPLQDRMFKFELTRRLDHDFGKVTKQEVKDFFRW |
| | | AKDHVVEVEHEFYVKKGGAKKRPAPSDADISEPKRVRESVA |
| | | QPSTSDAEASINYADRLARGHSL |
| 8 | Rep78 (wt) | MPGFYEIVIKVPSDLDEHLPGISDSFVNWVAEKEWELPPDSD |
| | | MDLNLIEQAPLTVAEKLQRDFLTEWRRVSKAPEALFFVQFEK |
| | | GESYFHMHVLVETTGVKSMVLGRFLSQIREKLIQRIYRGIEPT |
| | | LPNWFAVTKTRNGAGGGNKVVDECYIPNYLLPKTQPELQW |
| | | AWTNMEQYLSACLNLTERKRLVAQHLTHVSQTQEQNKENQ |
| | | NPNSDAPVIRSKTSARYMELVGWLVDKGITSEKQWIQEDQA |
| | | SYISFNAASNSRSQIKAALDNAGKIMSLTKTAPDYLVGQQPV |
| | | EDISSNRIYKILELNGYDPQYAASVFLGWATKKFGKRNTIWL |
| | | FGPATTGKTNIAEAIAHTVPFYGCVNWTNENFPFNDCVDKM |
| | | VIWWEEGKMTAKVVESAKAILGGSKVRVDQKCKSSAQIDPT |
| | | PVIVTSNTNMCAVIDGNSTTFEHQQPLQDRMFKFELTRRLDH |
| | | DFGKVTKQEVKDFFRWAKDHVVEVEHEFYVKKGGAKKRPA |
| | | PSDADISEPKRVRESVAQPSTSDAEASINYADRYQNKCSRHV |
| | | GMNLMLFPCRQCERMNQNSNICFTHGQKDCLECFPVSESQP |
| | | VSVVKKAYQKLCYIHHIMGKVPDACTACDLVNVDLDDCIFEQ |
| 9 | Rep68 (wt) | MPGFYEIVIKVPSDLDEHLPGISDSFVNWVAEKEWELPPDSD |
| | | MDLNLIEQAPLTVAEKLQRDFLTEWRRVSKAPEALFFVQFEK |
| | | GESYFHMHVLVETTGVKSMVLGRFLSQIREKLIQRIYRGIEPT |
| | | LPNWFAVTKTRNGAGGGNKVVDECYIPNYLLPKTQPELQW |
| | | AWTNMEQYLSACLNLTERKRLVAQHLTHVSQTQEQNKENQ |
| | | NPNSDAPVIRSKTSARYMELVGWLVDKGITSEKQWIQEDQA |
| | | SYISFNAASNSRSQIKAALDNAGKIMSLTKTAPDYLVGQQPV |
| | | EDISSNRIYKILELNGYDPQYAASVFLGWATKKFGKRNTIWL |
| | | FGPATTGKTNIAEAIAHTVPFYGCVNWTNENFPFNDCVDKM |
| | | VIWWEEGKMTAKVVESAKAILGGSKVRVDQKCKSSAQIDPT |
| | | PVIVTSNTNMCAVIDGNSTTFEHQQPLQDRMFKFELTRRLDH |
| | | DFGKVTKQEVKDFFRWAKDHVVEVEHEFYVKKGGAKKRPA |
| | | PSDADISEPKRVRESVAQPSTSDAEASINYADRLARGHSL |
| 10 | E2A (wt) | MASREEEQRETTPERGRGAARRPPTMEDVSSPSPSPPPPRAPP |
| | | KKRLRRRLESEDEEDSSQDALVPRTPSPRPSTSTADLAIASKK |
| | | KKKRPSPKPERPPSPEVIVDSEEEREDVALQMVGFSNPPVLIK |
| | | HGKGGKRTVRRLNEDDPVARGMRTQEEKEESSEAESESTVI |
| | | NPLSLPIVSAWEKGMEAARALMDKYHVDNDLKANFKLLPD |
| | | QVEALAAVCKTWLNEEHRGLQLTFTSNKTFVTMMGRFLQA |
| | | YLQSFAEVTYKHHEPTGCALWLHRCAEIEGELKCLHGSIMIN |
| | | KEHVIEMDVTSENGQRALKEQSSKAKIVKNRWGRNVVQISN |
| | | TDARCCVHDAACPANQFSGKSCGMFFSEGAKAQVAFKQIKA |
| | | FMQALYPNAQTGHGHLLMPLRCECNSKPGHAPFLGRQLPKL |
| | | TPFALSNAEDLDADLISDKSVLASVHHPALIVFQCCNPVYRN |
| | | SRAQGGGPNCDFKISAPDLLNALVMVRSLWSENFTELPRMV |
| | | VPEFKWSTKHQYRNVSLPVAHSDARQNPFDF |
| 11 | E4 ORF6 (wt) | MTTSGVPFGMTLRPTRSRLSRRTPYSRDRLPPFETETRATILE |
| | | DHPLLPECNTLTMHNVSYVRGLPCSVGFTLIQEWVVPWDMV |
| | | LTREELVILRKCMHVCLCCANIDIMTSMMIHGYESWALHCH |
| | | CSSPGSLQCIAGGQVLASWFRMVVDGAMFNQRFIWYREVV |
| | | NYNMPKEVMFMSSVFMRGRHLIYLRLWYDGHVGSVVPAMS |
| | | FGYSALHCGILNNIVVLCCSYCADLSEIRVRCCARRTRRLML |
| | | RAVRIIAEETTAMLYSCRTERRRQQFIRALLQHHRPILMHDY |
| | | DSTPM |
| 12 | E4 ORF6 | atgactacgtccggcgttccatttggcatgacactacgaccaacacgatctcggttgtctcggcgc |
| | (splice site | actccgtacagtagggatcgcctacctccttttgagacagagacccgcgctaccatactggagga |
| | removed) | tcatccgctgctgcccgaatgtaacactttgacaatgcacaaTgtTTCCtacgtgcgaggtctt |
| | | ccctgcagtgtgggatttacgctgattcaggaatgggttgttccctgggatatggttctgacgcgg |
| | | gaggagcttgtaatcctgaggaagtgtatgcacgtgtgcctgtgttgtgccaacattgatatcatga |
| | | cgagcatgatgatccatggttacgagtcctgggctctccactgtcattgttccagtcccggttccct |
| | | gcagtgcatagccggcgggcaggttttggccagctggtttaggatggtggtggatggcgccatg |
| | | tttaatcagaggtttatatggtaccgggaggtggtgaattacaacatgccaaaagaggtaatgttta |
| | | tgtccagcgtgtttatgaggggtcgccacttaatctacctgcgcttgtggtatgatggccacgtggg |
| | | ttctgtggtccccgccatgagctttggatacagcgccttgcactgtgggattttgaacaatattgtgg |
| | | tgctgtgctgcagttactgtgctgatttaagtgagatcagggtgcgctgctgtgcccggaggacaa |
| | | ggcgtctcatgctgcgggcggtgcgaatcatcgctgaggagaccactgccatgttgtattcctgc |
| | | aggacggagcggcggcggcagcagtttattegcgcgctgctgcagcaccaccgccctatoctg |
| | | atgcacgattatgactctacccccatgTAGtaa |
| 13 | VARNA | CGACGTAATCCGTAGATGTACCTGGACATCCAGGTGATGC |
| | | CGGCGGCGGTGGTGGAGGCGCGCGGAAAGTCGCGGACGC |
| | | GGTTCCAGATGTTGCGCAGCGGCAAAAAGTGCTCCATGGT |
| | | CGGGACGCTCTGGCCGGTGAGGCGTGCGCAGTCGTTGACG |
| | | CTCTAGACCGTGCAAAAGGAGAGCCTGTAAGCGGGCACTC |
| | | TTCCGTGGTCTGGTGGATAAATTCGCAAGGGTATCATGGC |
| | | GGACGACCGGGGTTCGAACCCCGGATCCGGCCGTCCGCCG |
| | | TGATCCATGCGGTTACCGCCCGCGTGTCGAACCCAGGTGT |
| | | GCGACGTCAGACAACGGGGGAGCGCTCCTTTTGGCTTCCT |
| | | TCCAGGCGCGGCGGCTGCTGCGCTAGCTTTTTTGGCCACT |
| | | GGCCGCGCGCGGCGTAAGCGGTTAGGCTGGAAAGCGAAA |
| | | GCATTAAGTGGCTCGCTCCCTGTAGCCGGAGGGTTATTTTC |
| | | CAAGGGTTGAGTCGCAGGACCCCCGGTTCGAGTCTCGGGC |
| | | CGGCCGGACTGCGGCGAACGGGGGTTTGCCTCCCCGTCAT |
| | | GCAAGACCCCGCTTGCAAATTCCTCCGGAAACAGGGACGA |
| | | GCCCCTTTTTTGCTTTTCCCAGATGCATCCGGTGCTGCGGC |
| | | AGATGCGCCCCCCTCCTCAGCAGCGGCAAGAGCAAGAGC |
| | | AGCGGCAGACATGCAGGGCACCCTCCCCTTCTCCTACCGC |
| | | GTCAGGAGGGGCAACATCC |
| 14 | VP1 (wt) | MAADGYLPDWLEDTLSEGIRQWWKLKPGPPPPKPAERHKD |
| | | DSRGLVLPGYKYLGPFNGLDKGEPVNEADAAALEHDKAYD |
| | | RQLDSGDNPYLKYNHADAEFQERLKEDTSFGGNLGRAVFQA |
| | | KKRVLEPLGLVEEPVKTAPGKKRPVEHSPVEPDSSSGTGKAG |
| | | QQPARKRLNFGQTGDADSVPDPQPLGQPPAAPSGLGTNTMA |
| | | TGSGAPMADNNEGADGVGNSSGNWHCDSTWMGDRVITTST |
| | | RTWALPTYNNHLYKQISSQSGASNDNHYFGYSTPWGYFDFN |
| | | RFHCHFSPRDWQRLINNNWGFRPKRLNFKLFNIQVKEVTQN |
| | | DGTTTIANNLTSTVQVFTDSEYQLPYVLGSAHQGCLPPFPAD |
| | | VFMVPQYGYLTLNNGSQAVGRSSFYCLEYFPSQMLRTGNNF |
| | | TFSYTFEDVPFHSSYAHSQSLDRLMNPLIDQYLYYLSRTNTPS |
| | | GTTTQSRLQFSQAGASDIRDQSRNWLPGPCYRQQRVSKTSA |
| | | DNNNSEYSWTGATKYHLNGRDSLVNPGPAMASHKDDEEKF |
| | | FPQSGVLIFGKQGSEKTNVDIEKVMITDEEEIRTTNPVATEQY |
| | | GSVSTNLQRGNRQAATADVNTQGVLPGMVWQDRDVYLQG |
| | | PIWAKIPHTDGHFHPSPLMGGFGLKHPPPQILIKNTPVPANPST |
| | | TFSAAKFASFITQYSTGQVSVEIEWELQKENSKRWNPEIQYTS |
| | | NYNKSVNVDFTVDTNGVYSEPRPIGTRYLTRNL |
| 15 | VP2 (wt) | TAPGKKRPVEHSPVEPDSSSGTGKAGQQPARKRLNFGQTGD |
| | | ADSVPDPQPLGQPPAAPSGLGTNTMATGSGAPMADNNEGAD |
| | | GVGNSSGNWHCDSTWMGDRVITTSTRTWALPTYNNHLYKQ |
| | | ISSQSGASNDNHYFGYSTPWGYFDFNRFHCHFSPRDWQRLIN |
| | | NNWGFRPKRLNFKLFNIQVKEVTQNDGTTTIANNLTSTVQVF |
| | | TDSEYQLPYVLGSAHQGCLPPFPADVFMVPQYGYLTLNNGS |
| | | QAVGRSSFYCLEYFPSQMLRTGNNFTFSYTFEDVPFHSSYAH |
| | | SQSLDRLMNPLIDQYLYYLSRTNTPSGTTTQSRLQFSQAGAS |
| | | DIRDQSRNWLPGPCYRQQRVSKTSADNNNSEYSWTGATKY |
| | | HLNGRDSLVNPGPAMASHKDDEEKFFPQSGVLIFGKQGSEKT |
| | | NVDIEKVMITDEEEIRTTNPVATEQYGSVSTNLQRGNRQAAT |
| | | ADVNTQGVLPGMVWQDRDVYLQGPIWAKIPHTDGHFHPSP |
| | | LMGGFGLKHPPPQILIKNTPVPANPSTTFSAAKFASFITQYSTG |
| | | QVSVEIEWELQKENSKRWNPEIQYTSNYNKSVNVDFTVDTN |
| | | GVYSEPRPIGTRYLTRNL |
| 16 | VP3 (wt) | MATGSGAPMADNNEGADGVGNSSGNWHCDSTWMGDRVIT |
| | | TSTRTWALPTYNNHLYKQISSQSGASNDNHYFGYSTPWGYF |
| | | DFNRFHCHFSPRDWQRLINNNWGFRPKRLNFKLFNIQVKEVT |
| | | QNDGTTTIANNLTSTVQVFTDSEYQLPYVLGSAHQGCLPPFP |
| | | ADVFMVPQYGYLTLNNGSQAVGRSSFYCLEYFPSQMLRTGN |
| | | NFTFSYTFEDVPFHSSYAHSQSLDRLMNPLIDQYLYYLSRTNT |
| | | PSGTTTQSRLQFSQAGASDIRDQSRNWLPGPCYRQQRVSKTS |
| | | ADNNNSEYSWTGATKYHLNGRDSLVNPGPAMASHKDDEEK |
| | | FFPQSGVLIFGKQGSEKTNVDIEKVMITDEEEIRTTNPVATEQ |
| | | YGSVSTNLQRGNRQAATADVNTQGVLPGMVWQDRDVYLQ |
| | | GPIWAKIPHTDGHFHPSPLMGGFGLKHPPPQILIKNTPVPANP |
| | | STTFSAAKFASFITQYSTGQVSVEIEWELQKENSKRWNPEIQY |
| | | TSNYNKSVNVDFTVDTNGVYSEPRPIGTRYLTRNL |
| 17 | AAP (wt) | LETQTQYLTPSLSDSHQQPPLVWELIRWLQAVAHQWQTITR |
| | | APTEWVIPREIGIAIPHGWATESSPPAPEPGPCPPTTTTSTNKFP |
| | | ANQEPRTTITTLATAPLGGILTSTDSTATFHHVTGKDSSTTTG |
| | | DSDPRDSTSSSLTFKSKRSRRMTVRRRLPITLPARFRCLLTRST |
| | | SSRTSSARRIKDASRRSQQTSSWCHSMDTSP |
| 18 | P2A (without | ATNFSLLKQAGDVEENPGP |
| | GSG) | |
| 19 | T2A (without | EGRGSLLTCGDVEENPGP |
| | GSG) | |
| 20 | WT Rep (nucleic | atgccggggttttacgagattgtgattaaggtccccagcgaccttgacgagcatctgcccggcatt |
| | acid sequence) | tctgacagctttgtgaactgggtggccgagaaggaatgggagttgccgccagattctgacatgga |
| | | tctgaatctgattgagcaggcacccctgaccgtggccgagaagctgcagcgcgactttctgacg |
| | | gaatggcgccgtgtgagtaaggccccggaggcccttttctttgtgcaatttgagaagggagaga |
| | | gctacttccacatgcacgtgctcgtggaaaccaccggggtgaaatccatggttttgggacgtttcc |
| | | tgagtcagattcgcgaaaaactgattcagagaatttaccgcgggatcgagccgactttgccaaac |
| | | tggttcgcggtcacaaagaccagaaatggcgccggaggcgggaacaaggtggtggatgagtg |
| | | ctacatccccaattacttgctccccaaaacccagcctgagctccagtgggcgtggactaatatgg |
| | | aacagtatttaagcgcctgtttgaatctcacggagcgtaaacggttggtggcgcagcatctgacg |
| | | cacgtgtcgcagacgcaggagcagaacaaagagaatcagaatcccaattctgatgcgccggtg |
| | | atcagatcaaaaacttcagccaggtacatggagctggtcgggtggctcgtggacaaggggatta |
| | | cctcggagaagcagtggatccaggaggaccaggcctcatacatctccttcaatgcggcctccaa |
| | | ctcgcggtcccaaatcaaggctgccttggacaatgcgggaaagattatgagcctgactaaaacc |
| | | gcccccgactacctggtgggccagcagcccgtggaggacatttccagcaatoggatttataaaat |
| | | tttggaactaaacgggtacgatccccaatatgcggcttccgtctttctgggatgggccacgaaaaa |
| | | gttcggcaagaggaacaccatctggctgtttgggcctgcaactaccgggaagaccaacatcgcg |
| | | gaggccatagcccacactgtgcccttctacgggtgcgtaaactggaccaatgagaactttcccttc |
| | | aacgactgtgtcgacaagatggtgatctggtgggaggaggggaagatgaccgccaaggtcgtg |
| | | gagtcggccaaagccattctcggaggaagcaaggtgcgcgtggaccagaaatgcaagtcctcg |
| | | gcccagatagacccgactcccgtgatcgtcacctccaacaccaacatgtgcgccgtgattgacg |
| | | ggaactcaacgaccttcgaacaccagcagccgttgcaagaccggatgttcaaatttgaactcacc |
| | | cgccgtctggatcatgactttgggaaggtcaccaagcaggaagtcaaagactttttccggtgggc |
| | | aaaggatcacgtggttgaggtggagcatgaattctacgtcaaaaagggtggagccaagaaaag |
| | | acccgcccccagtgacgcagatataagtgagcccaaacgggtgcgcgagtcagttgcgcagc |
| | | catcgacgtcagacgcggaagcttcgatcaactacgcagacaggtaccaaaacaaatgttctcgt |
| | | cacgtgggcatgaatctgatgctgtttccctgcagacaatgcgagagaatgaatcagaattcaaat |
| | | atctgcttcactcacggacagaaagactgtttagagtgctttcccgtgtcagaatctcaacccgtttc |
| | | tgtcgtcaaaaaggcgtatcagaaactgtgctacattcatcatatcatgggaaaggtgccagacg |
| | | cttgcactgcctgcgatctggtcaatgtggatttggatgactgcatctttgaacaataaatgatttaa |
| | | atcaggtatggctgccgatggttatcttccagattggctcgaggacactctctctga |
| 21 | WT Rep (amino | MPGFYEIVIKVPSDLDEHLPGISDSFVNWVAEKEWELPPDSD |
| | acid Sequence) | MDLNLIEQAPLTVAEKLQRDFLTEWRRVSKAPEALFFVQFEK |
| | | GESYFHMHVLVETTGVKSMVLGRFLSQIREKLIQRIYRGIEPT |
| | | LPNWFAVTKTRNGAGGGNKVVDECYIPNYLLPKTQPELQW |
| | | AWTNMEQYLSACLNLTERKRLVAQHLTHVSQTQEQNKENQ |
| | | NPNSDAPVIRSKTSARYMELVGWLVDKGITSEKQWIQEDQA |
| | | SYISFNAASNSRSQIKAALDNAGKIMSLTKTAPDYLVGQQPV |
| | | EDISSNRIYKILELNGYDPQYAASVFLGWATKKFGKRNTIWL |
| | | FGPATTGKTNIAEAIAHTVPFYGCVNWTNENFPFNDCVDKM |
| | | VIWWEEGKMTAKVVESAKAILGGSKVRVDQKCKSSAQIDPT |
| | | PVIVTSNTNMCAVIDGNSTTFEHQQPLQDRMFKFELTRRLDH |
| | | DFGKVTKQEVKDFFRWAKDHVVEVEHEFYVKKGGAKKRPA |
| | | PSDADISEPKRVRESVAQPSTSDAEASINYADRYQNKCSRHV |
| | | GMNLMLFPCRQCERMNQNSNICFTHGQKDCLECFPVSESQP |
| | | VSVVKKAYQKLCYIHHIMGKVPDACTACDLVNVDLDDCIFE |
| | | Q-MI-IRYGCRWLSSRLARGHSL- |
| 22 | DD-Rep78 | MISLIAALAVDYVIGMENAMPWNLPADLAWFKRNTLNKPVI |
| | [pGEX-2T] | MGRHTWESIGRPLPGRKNIILSSQPSTDDRVTWVKSVDEAIA |
| | | ACGDVPEIMVIGGGRVIEQFLPKAQKLYLTHIDAEVEGDTHF |
| | | PDYEPDDWESVFSEFHDADAQNSHSYCFEILERRSDLVPRGS |
| | | PGFYEIVIKVPSDLDEHLPGISDSFVNWVAEKEWELPPDSDM |
| | | DLNLIEQAPLTVAEKLQRDFLTEWRRVSKAPEALFFVQFEKG |
| | | ESYFHMHVLVETTGVKSMVLGRFLSQIREKLIQRIYRGIEPTL |
| | | PNWFAVTKTRNGAGGGNKVVDECYIPNYLLPKTQPELQWA |
| | | WTNMEQYLSACLNLTERKRLVAQHLTHVSQTQEQNKENQN |
| | | PNSDAPVIRSKTSARYMELVGWLVDKGITSEKQWIQEDQAS |
| | | YISFNAASNSRSQIKAALDNAGKIMSLTKTAPDYLVGQQPVE |
| | | DISSNRIYKILELNGYDPQYAASVFLGWATKKFGKRNTIWLF |
| | | GPATTGKTNIAEAIAHTVPFYGCVNWTNENFPFNDCVDKMV |
| | | IWWEEGKMTAKVVESAKAILGGSKVRVDQKCKSSAQIDPTP |
| | | VIVTSNTNMCAVIDGNSTTFEHQQPLQDRMFKFELTRRLDHD |
| | | FGKVTKQEVKDFFRWAKDHVVEVEHEFYVKKGGAKKRPAP |
| | | SDADISEPKRVRESVAQPSTSDAEASINYADRYQNKCSRHVG |
| | | MNLMLFPCRQCERMNQNSNICFTHGQKDCLECFPVSESQPVS |
| | | VVKKAYQKLCYIHHIMGKVPDACTACDLVNVDLDDCIFEQ |
| 23 | DD-Rep78 | MISLIAALAVDYVIGMENAMPWNLPADLAWFKRNTLNKPVI |
| | [linker | MGRHTWESIGRPLPGRKNIILSSQPSTDDRVTWVKSVDEAIA |
| | EAAAK | ACGDVPEIMVIGGGRVIEQFLPKAQKLYLTHIDAEVEGDTHF |
| | (SEQ ID | PDYEPDDWESVFSEFHDADAQNSHSYCFEILERRAEAAAKEA |
| | NO: 89)] | AAKEAAAKAPGFYEIVIKVPSDLDEHLPGISDSFVNWVAEKE |
| | | WELPPDSDMDLNLIEQAPLTVAEKLQRDFLTEWRRVSKAPE |
| | | ALFFVQFEKGESYFHMHVLVETTGVKSMVLGRFLSQIREKLI |
| | | QRIYRGIEPTLPNWFAVTKTRNGAGGGNKVVDECYIPNYLLP |
| | | KTQPELQWAWTNMEQYLSACLNLTERKRLVAQHLTHVSQT |
| | | QEQNKENQNPNSDAPVIRSKTSARYMELVGWLVDKGITSEK |
| | | QWIQEDQASYISFNAASNSRSQIKAALDNAGKIMSLTKTAPD |
| | | YLVGQQPVEDISSNRIYKILELNGYDPQYAASVFLGWATKKF |
| | | GKRNTIWLFGPATTGKTNIAEAIAHTVPFYGCVNWTNENFPF |
| | | NDCVDKMVIWWEEGKMTAKVVESAKAILGGSKVRVDQKC |
| | | KSSAQIDPTPVIVTSNTNMCAVIDGNSTTFEHQQPLQDRMFK |
| | | FELTRRLDHDFGKVTKQEVKDFFRWAKDHVVEVEHEFYVK |
| | | KGGAKKRPAPSDADISEPKRVRESVAQPSTSDAEASINYADR |
| | | YQNKCSRHVGMNLMLFPCRQCERMNQNSNICFTHGQKDCL |
| | | ECFPVSESQPVSVVKKAYQKLCYIHHIMGKVPDACTACDLV |
| | | NVDLDDCIFEQ |
| 24 | DD-Rep68 | MISLIAALAVDYVIGMENAMPWNLPADLAWFKRNTLNKPVI |
| | [pGEX-2T] | MGRHTWESIGRPLPGRKNIILSSQPSTDDRVTWVKSVDEAIA |
| | | ACGDVPEIMVIGGGRVIEQFLPKAQKLYLTHIDAEVEGDTHF |
| | | PDYEPDDWESVFSEFHDADAQNSHSYCFEILERRSDLVPRGS |
| | | PGFYEIVIKVPSDLDEHLPGISDSFVNWVAEKEWELPPDSDM |
| | | DLNLIEQAPLTVAEKLQRDFLTEWRRVSKAPEALFFVQFEKG |
| | | ESYFHMHVLVETTGVKSMVLGRFLSQIREKLIQRIYRGIEPTL |
| | | PNWFAVTKTRNGAGGGNKVVDECYIPNYLLPKTQPELQWA |
| | | WTNMEQYLSACLNLTERKRLVAQHLTHVSQTQEQNKENQN |
| | | PNSDAPVIRSKTSARYMELVGWLVDKGITSEKQWIQEDQAS |
| | | YISFNAASNSRSQIKAALDNAGKIMSLTKTAPDYLVGQQPVE |
| | | DISSNRIYKILELNGYDPQYAASVFLGWATKKFGKRNTIWLF |
| | | GPATTGKTNIAEAIAHTVPFYGCVNWTNENFPFNDCVDKMV |
| | | IWWEEGKMTAKVVESAKAILGGSKVRVDQKCKSSAQIDPTP |
| | | VIVTSNTNMCAVIDGNSTTFEHQQPLQDRMFKFELTRRLDHD |
| | | FGKVTKQEVKDFFRWAKDHVVEVEHEFYVKKGGAKKRPAP |
| | | SDADISEPKRVRESVAQPSTSDAEASINY ADRLARGHSL |
| 25 | DD-Rep68 | MISLIAALAVDYVIGMENAMPWNLPADLAWFKRNTLNKPVI |
| | [linker | MGRHTWESIGRPLPGRKNIILSSQPSTDDRVTWVKSVDEAIA |
| | EAAAK | ACGDVPEIMVIGGGRVIEQFLPKAQKLYLTHIDAEVEGDTHF |
| | (SEQ ID | PDYEPDDWESVFSEFHDADAQNSHSYCFEILERRAEAAAKEA |
| | NO: 89)] | AAKEAAAKAPGFYEIVIKVPSDLDEHLPGISDSFVNWVAEKE |
| | | WELPPDSDMDLNLIEQAPLTVAEKLQRDFLTEWRRVSKAPE |
| | | ALFFVQFEKGESYFHMHVLVETTGVKSMVLGRFLSQIREKLI |
| | | QRIYRGIEPTLPNWFAVTKTRNGAGGGNKVVDECYIPNYLLP |
| | | KTQPELQWAWTNMEQYLSACLNLTERKRLVAQHLTHVSQT |
| | | QEQNKENQNPNSDAPVIRSKTSARYMELVGWLVDKGITSEK |
| | | QWIQEDQASYISFNAASNSRSQIKAALDNAGKIMSLTKTAPD |
| | | YLVGQQPVEDISSNRIYKILELNGYDPQYAASVFLGWATKKF |
| | | GKRNTIWLFGPATTGKTNIAEAIAHTVPFYGCVNWTNENFPF |
| | | NDCVDKMVIWWEEGKMTAKVVESAKAILGGSKVRVDQKC |
| | | KSSAQIDPTPVIVTSNTNMCAVIDGNSTTFEHQQPLQDRMFK |
| | | FELTRRLDHDFGKVTKQEVKDFFRWAKDHVVEVEHEFYVK |
| | | KGGAKKRPAPSDADISEPKRVRESVAQPSTSDAEASINYADR |
| | | LARGHSL |
| 26 | DD-E2A | MISLIAALAVDYVIGMENAMPWNLPADLAWFKRNTLNKPVI |
| | | MGRHTWESIGRPLPGRKNIILSSQPSTDDRVTWVKSVDEAIA |
| | | ACGDVPEIMVIGGGRVIEQFLPKAQKLYLTHIDAEVEGDTHF |
| | | PDYEPDDWESVFSEFHDADAQNSHSYCFEILERRGSGASREE |
| | | EQRETTPERGRGAARRPPTMEDVSSPSPSPPPPRAPPKKRLRR |
| | | RLESEDEEDSSQDALVPRTPSPRPSTSTADLAIASKKKKKRPS |
| | | PKPERPPSPEVIVDSEEEREDVALQMVGFSNPPVLIKHGKGGK |
| | | RTVRRLNEDDPVARGMRTQEEKEESSEAESESTVINPLSLPIV |
| | | SAWEKGMEAARALMDKYHVDNDLKANFKLLPDQVEALAA |
| | | VCKTWLNEEHRGLQLTFTSNKTFVTMMGRFLQAYLQSFAEV |
| | | TYKHHEPTGCALWLHRCAEIEGELKCLHGSIMINKEHVIEMD |
| | | VTSENGQRALKEQSSKAKIVKNRWGRNVVQISNTDARCCVH |
| | | DAACPANQFSGKSCGMFFSEGAKAQVAFKQIKAFMQALYPN |
| | | AQTGHGHLLMPLRCECNSKPGHAPFLGRQLPKLTPFALSNAE |
| | | DLDADLISDKSVLASVHHPALIVFQCCNPVYRNSRAQGGGPN |
| | | CDFKISAPDLLNALVMVRSLWSENFTELPRMVVPEFKWSTK |
| | | HQYRNVSLPVAHSDARQNPFDF |
| 27 | DD-E4orf6 | MISLIAALAVDYVIGMENAMPWNLPADLAWFKRNTLNKPVI |
| | | MGRHTWESIGRPLPGRKNIILSSQPSTDDRVTWVKSVDEAIA |
| | | ACGDVPEIMVIGGGRVIEQFLPKAQKLYLTHIDAEVEGDTHF |
| | | PDYEPDDWESVFSEFHDADAQNSHSYCFEILERRGSGTTSGV |
| | | PFGMTLRPTRSRLSRRTPYSRDRLPPFETETRATILEDHPLLPE |
| | | CNTLTMHNVSYVRGLPCSVGFTLIQEWVVPWDMVLTREELV |
| | | ILRKCMHVCLCCANIDIMTSMMIHGYESWALHCHCSSPGSLQ |
| | | CIAGGQVLASWFRMVVDGAMFNQRFIWYREVVNYNMPKE |
| | | VMFMSSVFMRGRHLIYLRLWYDGHVGSVVPAMSFGYSALH |
| | | CGILNNIVVLCCSYCADLSEIRVRCCARRTRRLMLRAVRIIAE |
| | | ETTAMLYSCRTERRRQQFIRALLQHHRPILMHDYDSTPM |
| 28 | RfxCas13d | MSPKKKRKVEASIEKKKSFAKGMGVKSTLVSGSKVYMTTFA |
| | | EGSDARLEKIVEGDSIRSVNEGEAFSAEMADKNAGYKIGNAK |
| | | FSHPKGYAVVANNPLYTGPVQQDMLGLKETLEKRYFGESAD |
| | | GNDNICIQVIHNILDIEKILAEYITNAAYAVNNISGLDKDIIGFG |
| | | KFSTVYTYDEFKDPEHHRAAFNNNDKLINAIKAQYDEFDNFL |
| | | DNPRLGYFGQAFFSKEGRNYIINYGNECYDILALLSGLRHWV |
| | | VHNNEEESRISRTWLYNLDKNLDNEYISTLNYLYDRITNELT |
| | | NSFSKNSAANVNYIAETLGINPAEFAEQYFRFSIMKEQKNLGF |
| | | NITKLREVMLDRKDMSEIRKNHKVFDSIRTKVYTMMDFVIY |
| | | RYYIEEDAKVAAANKSLPDNEKSLSEKDIFVINLRGSFNDDQ |
| | | KDALYYDEANRIWRKLENIMHNIKEFRGNKTREYKKKDAPR |
| | | LPRILPAGRDVSAFSKLMYALTMFLDGKEINDLLTTLINKFDN |
| | | IQSFLKVMPLIGVNAKFVEEYAFFKDSAKIADELRLIKSFARM |
| | | GEPIADARRAMYIDAIRILGTNLSYDELKALADTFSLDENGN |
| | | KLKKGKHGMRNFIINNVISNKRFHYLIRYGDPAHLHEIAKNE |
| | | AVVKFVLGRIADIQKKQGQNGKNQIDRYYETCIGKDKGKSV |
| | | SEKVDALTKIITGMNYDQFDKKRSVIEDTGRENAEREKFKKII |
| | | SLYLTVIYHILKNIVNINARYVIGFHCVERDAQLYKEKGYDIN |
| | | LKKLEEKGFSSVTKLCAGIDETAPDKRKDVEKEMAERAKESI |
| | | DSLESANPKLYANYIKYSDEKKAEEFTRQINREKAKTALNAY |
| | | LRNTKWNVIIREDLLRIDNKTCTLFRNKAVHLEVARYVHAYI |
| | | NDIAEVNSYFQLYHYIMQRIIMNERYEKSSGKVSEYFDAVND |
| | | EKKYNDRLLKLLCVPFGYCIPRFKNLSIEALFDRNEAAKFDK |
| | | EKKKVSGNSGSGPKKKRKVAAAYPYDVPDYA* |
| 29 | RNA Guide | CCCAGGCTTTGAGTTGCACTCGC |
| | E2A_7 | |
| 30 | RNA Guide | TTTGACGTAGAATTCATGCTCCA |
| | Rep_9 | |
| 31 | RNA Guide | CATCCATTTCAATCACGTGCTCC |
| | E2A_3 | |
| 32 | RNA Guide E4_8 | AAAGTGTTACATTCGGGCAGCAG |
| 33 | RNA Guide | GAAGATAACCATCGGCAGCCATA |
| | Rep_2 | |
| 34 | RNA Guide | GTTCCATATTAGTCCACGCCCAC |
| | Rep_3 | |
| 35 | RNA Guide | AATTGCACAAAGAAAAGGGCCTC |
| | Rep_6 | |
| 36 | RNA Guide | ACCCAGTTCACAAAGCTGTCAGA |
| | Rep_7 | |
| 37 | RNA Guide | GAGTTCAAATTTGAACATCCGGT |
| | Rep_8 | |
| 38 | RNA Guide | AGATCACCATCTTGTCGACACAG |
| | Rep_10 | |
| 39 | miRE.shRNA. | CGACTTCTTAACCCAACAGAAGGCTCGAGAAGGTATATTG |
| | E2A.490 | CTGTTGACAGTGAGCGAaaagtgaaagcacggtgataaTAGTGAAGC |
| | | CACAGATGTAttatcaccgtgctttcactttcTGCCTACTGCCTCGGACT |
| | | TCAAGGGGC |
| 40 | miRE.shRNA. | CGACTTCTTAACCCAACAGAAGGCTCGAGAAGGTATATTG |
| | E2A.1119 | CTGTTGACAGTGAGCGAgctttcatgcaggcgctgtatTAGTGAAGCC |
| | | ACAGATGTAatacagcgcctgcatgaaagccTGCCTACTGCCTCGGAC |
| | | TTCAAGGGGC |
| 41 | miRE.shRNA. | CGACTTCTTAACCCAACAGAAGGCTCGAGAAGGTATATTG |
| | E2A.1391 | CTGTTGACAGTGAGCGAccccaactgegacttcaagatTAGTGAAGCC |
| | | ACAGATGTAatcttgaagtcgcagttggggcTGCCTACTGCCTCGGACT |
| | | TCAAGGGGC |
| 42 | miRE.shRNA.E4. | CGACTTCTTAACCCAACAGAAGGCTCGAGAAGGTATATTG |
| | 25 | CTGTTGACAGTGAGCGAgcatgacactacgaccaacacTAGTGAAGC |
| | | CACAGATGTAgtgttggtcgtagtgtcatgccTGCCTACTGCCTCGGAC |
| | | TTCAAGGGGC |
| 43 | miRE.shRNA.E4. | CGACTTCTTAACCCAACAGAAGGCTCGAGAAGGTATATTG |
| | 113 | CTGTTGACAGTGAGCGAcgctaccatactggaggatcaTAGTGAAGCC |
| | | ACAGATGTAtgatcctccagtatggtagcgcTGCCTACTGCCTCGGACT |
| | | TCAAGGGGC |
| 44 | miRE.shRNA.E4. | CGACTTCTTAACCCAACAGAAGGCTCGAGAAGGTATATTG |
| | 452 | CTGTTGACAGTGAGCGAcgccatgtttaatcagaggttTAGTGAAGCC |
| | | ACAGATGTAaacctctgattaaacatggcgcTGCCTACTGCCTCGGACT |
| | | TCAAGGGGC |
| 45 | miRE.shRNA. | CGACTTCTTAACCCAACAGAAGGCTCGAGAAGGTATATTG |
| | rep40.64 | CTGTTGACAGTGAGCGAaccaggcctcatacatctcctTAGTGAAGCC |
| | | ACAGATGTAaggagatgtatgaggcctggtcTGCCTACTGCCTCGGAC |
| | | TTCAAGGGGC |
| 46 | miRE.shRNA. | CGACTTCTTAACCCAACAGAAGGCTCGAGAAGGTATATTG |
| | rep40.140 | CTGTTGACAGTGAGCGAaaagattatgagcctgactaaTAGTGAAGCC |
| | | ACAGATGTAttagtcaggctcataatctttc TGCCTACTGCCTCGGACTT |
| | | CAAGGGGC |
| 47 | miRE.shRNA. | CGACTTCTTAACCCAACAGAAGGCTCGAGAAGGTATATTG |
| | rep40.648 | CTGTTGACAGTGAGCGAttgcaagaccggatgttcaaaTAGTGAAGCC |
| | | ACAGATGTAtttgaacatccggtcttgcaacTGCCTACTGCCTCGGACT |
| | | TCAAGGGGC |
| 48 | miRE.shRNA. | CGACTTCTTAACCCAACAGAAGGCTCGAGAAGGTATATTG |
| | VP3.16 | CTGTTGACAGTGAGCGAgcgcaccaatggcagacaataTAGTGAAGC |
| | | CACAGATGTAtattgtctgccattggtgcgccTGCCTACTGCCTCGGAC |
| | | TTCAAGGGGC |
| 49 | miRE.shRNA. | CGACTTCTTAACCCAACAGAAGGCTCGAGAAGGTATATTG |
| | VP3.549 | CTGTTGACAGTGAGCGAgcagtaggacgctcttcatttTAGTGAAGCC |
| | | ACAGATGTAaaatgaagagcgtcctactgccTGCCTACTGCCTCGGAC |
| | | TTCAAGGGGC |
| 50 | miRE.shRNA. | CGACTTCTTAACCCAACAGAAGGCTCGAGAAGGTATATTG |
| | VP3.1524 | CTGTTGACAGTGAGCGAgactttactgtggacactaatTAGTGAAGCC |
| | | ACAGATGTAattagtgtccacagtaaagtccTGCCTACTGCCTCGGACT |
| | | TCAAGGGGC |
| 51 | VanR operator | ATTGGATCCAAT |
| 52 | TtgR operator | TATTTACAAACAACCATGAATGTAAGTA |
| 53 | Gal4 UAS (for | CGGAGTACTGTCCTCCGA |
| | CID systems) | |
| 54 | PhIF operator | ATGATACGAAACGTACCGTATCGTTAAGGT |
| 55 | CymR operator | agaaacaaaccaacctgtctgtatta |
| | v1 | |
| 56 | CymR operator | aacaaacagacaatctggtctgtttgta |
| | v2 | |
| 57 | TetOff- | MSRLDKSKVINSALELLNEVGIEGLTTRKLAQKLGVEQPTLY |
| | Advanced | WHVKNKRALLDALAIEMLDRHHTHFCPLEGESWQDFLRNN |
| | | AKSFRCALLSHRDGAKVHLGTRPTEKQYETLENQLAFLCQQ |
| | | GFSLENALYALSAVGHFTLGCVLEDQEHQVAKEERETPTTDS |
| | | MPPLLRQAIELFDHQGAEPAFLFGLELIICGLEKQLKCESGGP |
| | | ADALDDFDLDMLPADALDDFDLDMLPADALDDFDLDMLPG |
| 58 | VanR-VP16 | MDMPRIKPGQRVMMALRKMIASGEIKSGERIAEIPTAAALGV |
| | | SRMPVRIALRSLEQEGLVVRLGARGYAARGVSSDQIRDAIEV |
| | | RGVLEGFAARRLAERGMTAETHARFVVLIAEGEALFAAGRL |
| | | NGEDLDRYAAYNQAFHDTLVSAAGNGAVESALARNGFEPF |
| | | AAAGALALDLMDLSAEYEHLLAAHRQHQAVLDAVSCGDAE |
| | | GAERIMRDHALAAIRNAKVFEAAASAGAPLGAAWSIRADSG |
| | | GGGPTDALDDFDLDMLPADALDDFDLDMLPADALDDFDLD |
| | | MLPGPPKKKRKV |
| 59 | TtgR-VP16 | MVRRTKEEAQETRAQIIEAAERAFYKRGVARTTLADIAELAG |
| | | VTRGAIYWHENNKAELVQALLDSLHETHDHLARASESEDEV |
| | | DPLGCMRKLLLQVFNELVLDARTRRINEILHHKCEFTDDMCE |
| | | IRQQHQSAVLDCHKGITLTLANVVRRGQLPGELDAERAAVA |
| | | MFAYVDGLIRRWLLLPDSVDLLGDVEKWVDTGLDMLRLSP |
| | | ALRKSGGGGPTDALDDFDLDMLPADALDDFDLDMLPADAL |
| | | DDFDLDMLPGPPKKKRKV |
| 60 | PhIF-VP16 | MARTPSRSSIGSLRSPHTHKAILTSTIEILKECGYSGLSIESVAR |
| | | RAGAGKPTIYRWWTNKAALIAEVYENEIEQVRKFPDLGSFK |
| | | ADLDFLLHNLWKVWRETICGEAFRCVIAEAQLDPVTLTQLK |
| | | DQFMERRREIPKKLVEDAISNGELPKDINRELLLDMIFGFCW |
| | | YRLLTEQLTVEQDIEEFTFLLINGVCPGTQCSGGGGPTDALDD |
| | | FDLDMLPADALDDFDLDMLPADALDDFDLDMLPGPPKKKR |
| | | KV |
| 61 | cTA | MSPKRRTQAERAMETQGKLIAAALGVLREKGYAGFRIADVP |
| | | GAAGVSRGAQSHHFPTKLELLLATFEWLYEQITERSRARLAK |
| | | LKPEDDVIQQMLDDAAEFFLDDDFSIGLDLIVAADRDPALRE |
| | | GIQRTVERNRFVVEDMWLGVLVSRGLSRDDAEDILWLIFNS |
| | | VRGLVVRSLWQKDKERFERVRNSTLEIARERYAKFKRSGGG |
| | | GPTDALDDFDLDMLPADALDDFDLDMLPADALDDFDLDML |
| | | PGPPKKKRKV |
| 62 | ODC | NPDFPPEVEEQDASTLPVSCAWESGMKRHRAACASASINV |
| 63 | ODC D12A | NPDFPPEVEEQAASTLPVSCAWESGMKRHRAACASASINV |
| 64 | ODC T15A | NPDFPPEVEEQDASALPVSCAWESGMKRHRAACASASINV |
| 65 | ODC C20A | NPDFPPEVEEQDASTLPVSAAWESGMKRHRAACASASINV |
| 66 | ODC S24A | NPDFPPEVEEQDASTLPVSCAWEAGMKRHRAACASASINV |
| 67 | shRNA.E2A.490 | aaagtgaaagcacggtgataaNNNNNttatcaccgtgctttcactttc |
| 68 | shRNA.E2A.1119 | gctttcatgcaggcgctgtatNNNNNatacagcgcctgcatgaaagcc |
| 69 | shRNA.E2A.1391 | ccccaactgcgacttcaagatNNNNNatcttgaagtcgcagttggggc |
| 70 | shRNA.E4.25 | gcatgacactacgaccaacacNNNNNgtgttggtcgtagtgtcatgcc |
| 71 | shRNA.E4.113 | cgctaccatactggaggatcaNNNNNtgatcctccagtatggtagcgc |
| 72 | shRNA.E4.452 | cgccatgtttaatcagaggttNNNNNaacctctgattaaacatggcgc |
| 73 | shRNA.rep40.64 | accaggcctcatacatctcctNNNNNaggagatgtatgaggcctggtc |
| 74 | shRNA.rep40.140 | aaagattatgagcctgactaaNNNNNttagtcaggctcataatctttc |
| 75 | shRNA.rep40.648 | ttgcaagaccggatgttcaaaNNNNNtttgaacatccggtcttgcaac |
| 76 | shRNA.VP3.16 | gcgcaccaatggcagacaataNNNNNtattgtctgccattggtgcgcc |
| 77 | shRNA.VP3.549 | gcagtaggacgctcttcatttNNNNNaaatgaagagcgtcctactgcc |
| 78 | shRNA.VP3.1524 | gactttactgtggacactaatNNNNNattagtgtccacagtaaagtcc |
| 79 | rcTA | MVIMSPKRRTQAERAMETQGKLIAAALGVLREKGYAGFRIA |
| | | DVPGAAGVSRGAQSHHFPTKLELLLATFEWLYEQITERSRAR |
| | | LAKLKPEDDVIQQMLDDAAEFFLDDDFSIGLDLIVAADRDPV |
| | | LREGIQRTVERNRFVVGDIWLGVLVSRGLSRDDAEDILWLIF |
| | | NSVRGLVVRSLWQKDKERFERVRNSTLEIARERYAKFKRSG |
| | | GGGPTDALDDFDLDMLPADALDDFDLDMLPADALDDFDLD |
| | | MLPGPPKKKRKV |
| 80 | MAAP (wt) | LAHHHQSPQSGIRTTAGVLCFLGTSTSDPSTDSTRESRSTRQT |
| | | PRPSSTTKPTTGSSTAETTRTSSTTTPTRSFRSALKKIRLLGATS |
| | | DEQSSRRKRGFLNLWAWLRNLLRRLREKRGR |
| 81 | ecDHFR | MISLIAALAVDYVIGMENAMPWNLPADLAWFKRNTLNKPVI |
| | | MGRHTWESIGRPLPGRKNIILSSQPSTDDRVTWVKSVDEAIA |
| | | ACGDVPEIMVIGGGRVIEQFLPKAQKLYLTHIDAEVEGDTHF |
| | | PDYEPDDWESVFSEFHDADAQNSHSYCFEILERR |
| 82 | DD-Rep52 | MISLIAALAVDYVIGMENAMPWNLPADLAWFKRNTLNKPVI |
| | | MGRHTWESIGRPLPGRKNIILSSQPSTDDRVTWVKSVDEAIA |
| | | ACGDVPEIMVIGGGRVIEQFLPKAQKLYLTHIDAEVEGDTHF |
| | | PDYEPDDWESVFSEFHDADAQNSHSYCFEILERRGSGMELVG |
| | | WLVDKGITSEKQWIQEDQASYISFNAASNSRSQIKAALDNAG |
| | | KIMSLTKTAPDYLVGQQPVEDISSNRIYKILELNGYDPQYAAS |
| | | VFLGWATKKFGKRNTIWLFGPATTGKTNIAEAIAHTVPFYGC |
| | | VNWTNENFPFNDCVDKMVIWWEEGKMTAKVVESAKAILGG |
| | | SKVRVDQKCKSSAQIDPTPVIVTSNTNMCA VIDGNSTTFEHQ |
| | | QPLQDRMFKFELTRRLDHDFGKVTKQEVKDFFRWAKDHVV |
| | | EVEHEFYVKKGGAKKRPAPSDADISEPKRVRESVAQPSTSDA |
| | | EASINYADRYQNKCSRHVGMNLMLFPCRQCERMNQNSNICF |
| | | THGQKDCLECFPVSESQPVSVVKKAYQKLCYIHHIMGKVPD |
| | | ACTACDLVNVDLDDCIFEQ |
| 83 | DD-Rep40 | MISLIAALAVDYVIGMENAMPWNLPADLAWFKRNTLNKPVI |
| | | MGRHTWESIGRPLPGRKNIILSSQPSTDDRVTWVKSVDEAIA |
| | | ACGDVPEIMVIGGGRVIEQFLPKAQKLYLTHIDAEVEGDTHF |
| | | PDYEPDDWESVFSEFHDADAQNSHSYCFEILERRGSGMELVG |
| | | WLVDKGITSEKQWIQEDQASYISFNAASNSRSQIKAALDNAG |
| | | KIMSLTKTAPDYLVGQQPVEDISSNRIYKILELNGYDPQYAAS |
| | | VFLGWATKKFGKRNTIWLFGPATTGKTNIAEAIAHTVPFYGC |
| | | VNWTNENFPFNDCVDKMVIWWEEGKMTAKVVESAKAILGG |
| | | SKVRVDQKCKSSAQIDPTPVIVTSNTNMCAVIDGNSTTFEHQ |
| | | QPLQDRMFKFELTRRLDHDFGKVTKQEVKDFFRWAKDHVV |
| | | EVEHEFYVKKGGAKKRPAPSDADISEPKRVRESVAQPSTSDA |
| | | EASINYADRLARGHSL |
| 84 | DD-AAP | MISLIAALAVDYVIGMENAMPWNLPADLAWFKRNTLNKPVI |
| | | MGRHTWESIGRPLPGRKNIILSSQPSTDDRVTWVKSVDEAIA |
| | | ACGDVPEIMVIGGGRVIEQFLPKAQKLYLTHIDAEVEGDTHF |
| | | PDYEPDDWESVFSEFHDADAQNSHSYCFEILERRGSGMETQT |
| | | QYLTPSLSDSHQQPPLVWELIRWLQAVAHQWQTITRAPTEW |
| | | VIPREIGIAIPHGWATESSPPAPEPGPCPPTTTTSTNKFPANQEP |
| | | RTTITTLATAPLGGILTSTDSTATFHHVTGKDSSTTTGDSDPR |
| | | DSTSSSLTFKSKRSRRMTVRRRLPITLPARFRCLLTRSTSSRTS |
| | | SARRIKDASRRSQQTSSWCHSMDTSP |
| 85 | DiCas7-11 | MTTTMKISIEFLEPFRMTKWQESTRRNKNNKEFVRGQAFAR |
| | | WHRNKKDNTKGRPYITGTLLRSAVIRSAENLLTLSDGKISEK |
| | | TCCPGKFDTEDKDRLLQLRQRSTLRWTDKNPCPDNAETYCP |
| | | FCELLGRSGNDGKKAEKKDWRFRIHFGNLSLPGKPDFDGPK |
| | | AIGSQRVLNRVDFKSGKAHDFFKAYEVDHTRFPRFEGEITID |
| | | NKVSAEARKLLCDSLKFTDRLCGALCVIRFDEYTPAADSGKQ |
| | | TENVQAEPNANLAEKTAEQIISILDDNKKTEYTRLLADAIRSL |
| | | RRSSKLVAGLPKDHDGKDDHYLWDIGKKKKDENSVTIRQIL |
| | | TTSADTKELKNAGKWREFCEKLGEALYLKSKDMSGGLKITR |
| | | RILGDAEFHGKPDRLEKSRSVSIGSVLKETVVCGELVAKTPFF |
| | | FGAIDEDAKQTDLQVLLTPDNKYRLPRSAVRGILRRDLQTYF |
| | | DSPCNAELGGRPCMCKTCRIMRGITVMDARSEYNAPPEIRHR |
| | | TRINPFTGTVAEGALFNMEVAPEGIVFPFQLRYRGSEDGLPD |
| | | ALKTVLKWWAEGQAFMSGAASTGKGRFRMENAKYETLDLS |
| | | DENQRNDYLKNWGWRDEKGLEELKKRLNSGLPEPGNYRDP |
| | | KWHEINVSIEMASPFINGDPIRAAVDKRGTDVVTFVKYKAEG |
| | | EEAKPVCAYKAESFRGVIRSAVARIHMEDGVPLTELTHSDCE |
| | | CLLCQIFGSEYEAGKIRFEDLVFESDPEPVTFDHVAIDRFTGG |
| | | AADKKKFDDSPLPGSPARPLMLKGSFWIRRDVLEDEEYCKA |
| | | LGKALADVNNGLYPLGGKSAIGYGQVKSLGIKGDDKRISRL |
| | | MNPAFDETDVAVPEKPKTDAEVRIEAEKVYYPHYFVEPHKK |
| | | VEREEKPCGHQKFHEGRLTGKIRCKLITKTPLIVPDTSNDDFF |
| | | RPADKEARKEKDEYHKSYAFFRLHKQIMIPGSELRGMVSSV |
| | | YETVTNSCFRIFDETKRLSWRMDADHQNVLQDFLPGRVTAD |
| | | GKHIQKFSETARVPFYDKTQKHFDILDEQEIAGEKPVRMWV |
| | | KRFIKRLSLVDPAKHPQKKQDNKWKRRKEGIATFIEQKNGSY |
| | | YFNVVTNNGCTSFHLWHKPDNFDQEKLEGIQNGEKLDCWV |
| | | RDSRYQKAFQEIPENDPDGWECKEGYLHVVGPSKVEFSDKK |
| | | GDVINNFQGTLPSVPNDWKTIRTNDFKNRKRKNEPVFCCED |
| | | DKGNYYTMAKYCETFFFDLKENEEYEIPEKARIKYKELLRVY |
| | | NNNPQAVPESVFQSRVARENVEKLKSGDLVYFKHNEKYVED |
| | | IVPVRISRTVDDRMIGKRMSADLRPCHGDWVEDGDLSALNA |
| | | YPEKRLLLRHPKGLCPACRLFGTGSYKGRVRFGFASLENDPE |
| | | WLIPGKNPGDPFHGGPVMLSLLERPRPTWSIPGSDNKFKVPG |
| | | RKFYVHHHAWKTIKDGNHPTTGKAIEQSPNNRTVEALAGGN |
| | | SFSFEIAFENLKEWELGLLIHSLQLEKGLAHKLGMAKSMGFG |
| | | SVEIDVESVRLRKDWKQWRNGNSEIPNWLGKGFAKLKEWF |
| | | RDELDFIENLKKLLWFPEGDQAPRVCYPMLRKKDDPNGNSG |
| | | YEELKDGEFKKEDRQKKLTTPWTPWA |

What is claimed is:

1. An engineered cell for AAV production, comprising one or more stably integrated nucleic acid molecules collectively comprising nucleic acid sequences encoding for: Rep52 or Rep40; DD-Rep78 or DD-Rep68; DD-E2A; DD-E4Orf6; VARNA; VP1; VP2; VP3; AAP; each of which is operably linked to a promoter; wherein:
(i) DD-E2A comprises the amino acid sequence of E2A linked directly or indirectly to a molecule binding degradation domain;
(ii) DD-E4orf6 comprises the amino acids sequence of E4orf6 linked directly or indirectly to a molecule binding degradation domain;
(iii) DD-Rep78 comprises the amino acid sequence of Rep78 linked directly or indirectly to a molecule binding degradation domain; and
(iv) DD-Rep68 comprises the amino acid sequence of Rep68 linked directly or indirectly to a molecule binding degradation domain.

2. The engineered cell of claim 1, wherein the degradation domain is selected from the group consisting of FKBP or ecDHFR degradation domains.

3. The engineered cell of claim 2, wherein the ecDHFR degradation domain is stabilized by the small molecules Trimethoprim.

4. The engineered cell of claim 2 or claim 3, wherein the FKBP degradation domain is stabilized by the small molecules Shield, Shield2 or SLF*.

5. The engineered cell of any one of claims 1-4, wherein the one or more stably integrated nucleic acid molecules comprises a first stably integrated nucleic acid molecule comprising the nucleic acid sequence encoding for Rep52 or Rep40, the nucleic acid sequence encoding for DD-Rep78 or DD-Rep68, the nucleic acid sequence encoding for VP1, the nucleic acid sequence encoding for VP2, the nucleic acid sequence encoding for VP3, and the nucleic acid sequence encoding for AAP.

6. The engineered cell of claim 5, wherein the first stably integrated nucleic acid molecule further comprises a selection marker that is operably linked to a promoter.

7. The engineered cell of claim 5 or claim 6, wherein the one or more stably integrated nucleic acid molecules comprises a second stably integrated nucleic acid molecule comprising the nucleic acid sequence encoding for DD-E2A, the nucleic acid sequence encoding for DD-E4orf6, and the nucleic acid sequence encoding for VARNA.

8. The engineered cell of claim 7, wherein the second stably integrated nucleic acid molecule further comprises a selection marker that is operably linked to a promoter.

9. The engineered cell of any one of claims 1-8, wherein the engineered cell comprises a nucleic acid sequence encoding for Rep52, wherein the nucleic acid sequence encoding for Rep52 comprises the amino acid sequence of SEQ ID NO: 6.

10. The engineered cell of any one of claims 1-8, wherein the engineered cell comprises a nucleic acid sequence encoding for Rep40, wherein the nucleic acid sequence encoding for Rep40 comprises the amino acid sequence of SEQ ID NO: 7.

11. The engineered cell of any one of claims 1-10, wherein the engineered cell comprises a nucleic acid sequence encoding for DD-Rep78, wherein the nucleic acid sequence encoding for DD-Rep78 the amino acid sequence of SEQ ID NO: 22 or SEQ ID NO: 23.

12. The engineered cell of any one of claims 1-10, wherein the engineered cell comprises a nucleic acid sequence encoding for DD-Rep68, wherein the nucleic acid sequence encoding for DD-Rep68 comprises the amino acid sequence of SEQ ID NO: 24 or SEQ ID NO: 25.

13. The engineered cell of any one of claims 1-12, wherein the nucleic acid sequence encoding for VP1 comprises the amino acid sequence of SEQ ID NO: 14.

14. The engineered cell of any one of claim 1-13, wherein the nucleic acid sequence encoding for VP2 comprises the amino acid sequence of SEQ ID NO: 15.

15. The engineered cell of any one of claims 1-14, wherein the nucleic acid sequence encoding for VP3 comprises the amino acid sequence of SEQ ID NO: 16.

16. The engineered cell of any one of claims 1-15, wherein the nucleic acid sequence encoding for AAP comprises the amino acid sequence of SEQ ID NO: 17.

17. The engineered cell of any one of claims 1-16, wherein the nucleic acid sequence encoding for VARNA comprises the nucleic acid sequence of SEQ ID NO: 13.

18. The engineered cell of any one of claims 1-17, wherein the nucleic acid sequence encoding for DD-E2A comprises the amino acid sequence of SEQ ID NO: 26.

19. The engineered cell of any one of claims 1-18, wherein the nucleic acid sequence encoding for DD-E4orf6 comprises the amino acid sequence of SEQ ID NO: 27.

20. A kit comprising the engineered cell of any one of claims 1-19.

21. The kit of claim 20 further comprising a nucleic acid molecule comprising, from 5′ to 3′: (i) a nucleic acid sequence of a 5′ inverted tandem repeat; (ii) a multiple cloning site; and (iii) a nucleic acid sequence of a 3′ inverted tandem repeat.

22. The kit of claim 21, wherein the nucleic acid molecule is a plasmid or a vector.

23. A method for AAV production, comprising culturing the engineered cell of any one of claims 1-19 with a molecule capable of binding the molecule binding degradation domain.

24. The method of claim 23, wherein the molecule is Trimethoprim or Shield.

25. The method of claim 23 or claim 24, wherein culturing the engineered cell with the molecule increases the concentration of proteins comprising the degradation domain.

26. An engineered cell for AAV production, comprising one or more stably integrated nucleic acid molecules collectively comprising a nucleic acid sequence encoding for each of: Rep52 or Rep40; Rep78 or Rep68; E2A; E4Orf6; VARNA; VP1; VP2; VP3; AAP; one or more crRNAs; and Cas13 or Cas7-11, wherein the nucleic acid sequences of one or more of Rep52 or Rep40, Rep78 or Rep68, E2A, and E4Orf6 further comprises a nucleic acid sequence that complements at least one of the one or more crRNAs.

27. The engineered cell of claim 26, wherein the one or more stably integrated nucleic acid molecules comprises a first stably integrated nucleic acid molecule comprising the nucleic acid sequence encoding Cas13.

28. The engineered cell of claim 27, wherein the nucleic acid sequence encoding Cas13 encodes RfxCas13d.

29. The engineered cell of claim 28, wherein the nucleic acid sequence encoding RfxCas13d comprises SEQ ID NO: 28.

30. The engineered cell of any one of claims 27-29, wherein the nucleic acid sequence encoding Cas13 further comprises a degradation domain.

31. The engineered cell of claim 30, wherein the degradation domain is the c-terminal domain of ornithine decarboxylase (ODC) degradation domain, the M-ODC degradation domain containing an alanine substitution at amino acid position 12, 15, 20 or 24 (SEQ ID NOs: 62-66) or the auxin-inducible degron.

32. The engineered cell of claim 30 or claim 31, wherein the degradation domain is downstream of Cas13.

33. The engineered cell of any one of claims 27-32, wherein the nucleic acid sequence encoding Cas13 is operably linked to a first chemically inducible promoter.

34. The engineered cell of claim 33, wherein the first chemically inducible promoter is selected from the group consisting of pTRE3G, pTREtight, and a promoters containing at least one of VanR, TtgR, PhIF, or CymR, or the Gal4 UAS operator sequences

35. The engineered cell of claim 34, wherein the nucleic acid sequence encoding the first chemically inducible promoter is any one of SEQ ID NO: 1-3 or comprises any one of SEQ ID NOs: 51-56.

36. The engineered cell of claim 26, wherein the one or more stably integrated nucleic acid molecules comprises a first stably integrated nucleic acid molecule comprising the nucleic acid sequence encoding Cas7-11.

37. The engineered cell of claim 36, wherein the nucleic acid sequence encoding Cas7-11 encodes DiCas7-11.

38. The engineered cell of claim 37, wherein the nucleic acid sequence encoding DiCas7-11 comprises SEQ ID NO: 85.

39. The engineered cell of any one of claims 36-38, wherein the nucleic acid sequence encoding Cas7-11 further comprises a degradation domain.

40. The engineered cell of claim 39, wherein the degradation domain is the c-terminal domain of ornithine decarboxylase (ODC) degradation domain, the M-ODC degradation domain containing an alanine substitution at amino acid position 12, 15, 20 or 24 (SEQ ID NOs: 62-66), or the auxin-inducible degron.

41. The engineered cell of claim 39 or claim 40, wherein the degradation domain is downstream of Cas7-11.

42. The engineered cell of any one of claims 36-41, wherein the nucleic acid sequence encoding Cas7-11 is operably linked to a first chemically inducible promoter.

43. The engineered cell of claim 42, wherein the first chemically inducible promoter is selected from the group consisting of pTRE3G, pTREtight, and a promoters containing at least one of VanR, TtgR, PhIF, or CymR, or the Gal4 UAS operator sequences

44. The engineered cell of claim 43, wherein the nucleic acid sequence encoding the first chemically inducible promoter is any one of SEQ ID NO: 1-3 or comprises any one of SEQ ID NOs: 51-56.

45. The engineered cell of any one of claims 27-44, wherein the first stably integrated nucleic acid molecule further comprises a selection marker that is operably linked to a promoter.

46. The engineered cell of any one of claims 27-45, wherein the one or more stably integrated nucleic acid molecules comprises a second stably integrated nucleic acid molecule comprising the nucleic acid sequence encoding one or more crRNAs.

47. The engineered cell of claim 46, wherein the one or more crRNAs each comprise a nucleic acid sequence that is complementary to any one of the nucleic acid sequences encoding for Rep52 or Rep40, Rep78 or Rep68, E2A, and E4Orf6.

48. The engineered cell of claim 46 or claim 47, wherein the one or more crRNAs each comprise a nucleic acid sequence of any one of SEQ ID NOs: 29-38.

49. The engineered cell of any one of claims 46-48, wherein the nucleic acid sequence encoding for the one or more crRNAs is operably linked to a second chemically inducible promoter.

50. The engineered cell of claim 49, wherein the second chemically inducible promoter is selected from the group consisting of pTRE3G, pTREtight, or a promoter containing at least one of VanR, TtgR, PhIF, or CymR, or the Gal4 UAS operator sequences.

51. The engineered cell of claim 50, wherein the nucleic acid sequence encoding the second chemically inducible promoter is any one of SEQ ID NOs: 1-2 or comprises any one of SEQ ID NOs: 51-56.

52. The engineered cell of any one of claims 46-51, wherein the second stably integrated nucleic acid molecule further comprises a selection marker that is operably linked to a promoter.

53. The engineered cell of any one of claims 46-52, wherein the one or more stably integrated nucleic acid molecules comprises a third stably integrated nucleic acid molecule wherein the third stably integrated nucleic acid molecule comprises the nucleic acid sequence encoding for Rep52 or Rep40.

54. The engineered cell of claim 53, wherein the nucleic acid sequence encoding for Rep52 comprises an amino acid sequence of SEQ ID NO: 6.

55. The engineered cell of claim 53 or claim 54, wherein the nucleic acid sequence encoding for Rep40 comprises an amino acid sequence of SEQ ID NO: 7.

56. The engineered cell of any one of claims 53-55, wherein the third stably integrated nucleic acid molecule comprises a nucleic acid sequence encoding for Rep78 or Rep68.

57. The engineered cell of claim 56, wherein the nucleic acid sequence encoding for Rep78 comprises an amino acid sequence of SEQ ID NO: 8.

58. The engineered cell of claim 56 or claim 57, wherein the nucleic acid sequence encoding for Rep68 comprises an amino acid sequence of SEQ ID NO: 9.

59. The engineered cell of any one of claims 53-58, wherein the third stably integrated nucleic acid molecule comprises nucleic acid sequences encoding for VP1, VP2, and VP3.

60. The engineered cell of claim 59, wherein the nucleic acid sequence encoding for VP1 comprises the amino acid sequence of SEQ ID NO: 14.

61. The engineered cell of claim 59 or claim 60, wherein the nucleic acid sequence encoding for VP2 comprises the amino acid sequence of SEQ ID NO: 15.

62. The engineered cell of any one of claims 59-61, wherein the nucleic acid sequence encoding for VP3 comprises the amino acid sequence of SEQ ID NO: 16.

63. The engineered cell of any one of claims 53-62, wherein the third stably integrated nucleic acid molecule comprises a nucleic acid sequence encoding for AAP.

64. The engineered cell of claim 63, wherein the nucleic acid sequence encoding for AAP comprises the amino acid sequence of SEQ ID NO: 17.

65. The engineered cell of any one of claims 53-64, wherein the one or more stably integrated nucleic acid molecules comprises a fourth stably integrated nucleic acid molecule comprising the nucleic acid sequences encoding for E2A, E4Orf6, and VARNA.

66. The engineered cell of claim 65, wherein the nucleic acid sequence encoding for E2A comprises the amino acid sequence of SEQ ID NO: 10.

67. The engineered cell of claim 65 or claim 66, wherein the nucleic acid sequence encoding for E4Orf6 comprises the amino acid sequence of SEQ ID NO: 11.

68. The engineered cell of any one of claims 65-67, wherein the nucleic acid sequence encoding for VARNA comprises the nucleic acid sequence of SEQ ID NO: 13.

69. The engineered cell of any one of claims 65-68, wherein the fourth stably integrated nucleic acid molecule further comprises a selection marker that is operably linked to a promoter.

70. The engineered cell of any one of claims 65-69, wherein the nucleic acid sequence encoding for Cas13 or Cas7-11 and/or the nucleic acid sequence encoding for one or more crRNAs is operably linked to a chemically inducible promoter, and wherein the engineered cell further comprises a fifth stably integrated nucleic acid molecule, wherein the fifth stably integrated nucleic acid molecule comprises a nucleic acid sequence encoding for a transcriptional activator that, when expressed in the presence of a small molecule inducer, binds to the chemically inducible promoter of the engineered cell.

71. The engineered cell of claim 70, wherein the transcriptional activator is selected from the group consisting of TetOff, TetOff-Advanced, VanR-VP16, TtgR-VP16, PhIF-VP16, and the cumate-responsive transactivators cTA and rcTA.

72. The engineered cell of claim 70 or claim 71, wherein the small molecule inducer is selected from the group consisting of lactose, arabinose, and doxycycline.

73. The engineered cell of any one of claims 70-72, wherein the transcriptional activator is TetOff and the small molecule inducer is doxycycline.

74. The engineered cell of any one of claims 26-73, wherein the engineered cell is HEK293 cell or HeLa cell.

75. A kit comprising the engineered cell of any one of claims 26-74.

76. The kit of claim 75, further comprising a nucleic acid molecule comprising, from 5′ to 3′: (i) a nucleic acid sequence of a 5′ inverted tandem repeat; (ii) a multiple cloning site; and (iii) a nucleic acid sequence of a 3′ inverted tandem repeat.

77. The kit of claim 76, wherein the nucleic acid molecule is a plasmid or a vector.

78. A method for AAV production, comprising culturing the engineered cell of any one of claim 30-32 or 39-41 in the presence of a molecule that binds to the degradation domain and increases degradation of the degradation domain, thereby inducing AAV production, optionally wherein the molecule is Auxin or antizyme.

79. A method for AAV production, comprising culturing the engineered cell of claim 30 or 39 in the presence of a molecule that binds to the degradation domain and decreases degradation of the degradation domain and then culturing the engineered cell in the absence of the molecule, thereby inducing AAV production, wherein the degradation domain is selected from the group consisting of FKBP or ecDHFR, and optionally wherein the molecule is trimethoprim, Shield, Shield2 or SLF*.

80. A method for AAV production, comprising culturing the engineered cell of any one of claims 70-73 in the presence of the small molecule inducer corresponding to the transcriptional activator that binds to the chemically inducible promoter, thereby inducing AAV production.

81. The method of claim 80, wherein the small molecule inducer is selected from the group consisting of lactose, arabinose, and doxycycline.

82. An engineered cell for AAV production, comprising one or more nucleic acid molecules collectively comprising a nucleic acid sequence encoding for each of: Rep78 or Rep68; Rep52 or Rep40; VP1; VP2; VP3; E2A; E4orf6; VARNA; and shRNAs each of which is operably linked to a promoter, wherein one or more shRNAs comprise a sequence that is complementary to the mRNA of one or more of Rep78 or Rep68; Rep52 or Rep40; VP1; VP2; VP3; AAP; E2A; E4orf6; and VARNA.

83. The engineered cell of claim 82, wherein the one or more nucleic acid molecules comprises a first nucleic acid molecule comprising the nucleic acid sequence encoding for one or more shRNAs.

84. The engineered cell of claim 83, wherein the one or more shRNAs each comprise a nucleic acid sequence that is complementary to any one of the nucleic acid sequences encoding for Rep78 or Rep68; Rep52 or Rep40; VP1; VP2; VP3; AAP; E2A; E4orf6; and VARNA.

85. The engineered cell of claim 83 or claim 84, wherein the one or more shRNAs comprise a nucleic acid sequence of any one of SEQ ID NO: 39-50.

86. The engineered cell of any one of claims 83-85, wherein the nucleic acid sequence encoding for the one or more shRNAs is operably linked to a chemically inducible promoter.

87. The engineered cell of claim 86, wherein the chemically inducible promoter is selected from the group consisting of pTRE3G, pTREtight, or a promoter containing at least one of VanR, TtgR, PhIF, or CymR, or the Gal4 UAS operator sequences.

88. The engineered cell of claim 87, wherein the nucleic acid sequence encoding the chemically inducible promoter is any one of SEQ ID NOs: 1-2 or comprises any one of SEQ ID NOs: 51-56.

89. The engineered cell of any one of claims 83-88, wherein the first nucleic acid molecule further comprises a nucleic acid sequence encoding for a selection marker that is operably linked to a promoter.

90. The engineered cell of any one of claims 83-89, wherein the first nucleic acid molecule further comprises a nucleic acid sequence encoding for a Neo-TagBFP.

91. The engineered cell of any one of claims 83-89, wherein the first nucleic acid molecule is a plasmid or a vector.

92. The engineered cell of any one of claims 83-89, wherein the first nucleic acid molecule is stably integrated.

93. The engineered cell of any one of claims 83-92, wherein the one or more stably integrated nucleic acid molecules comprises a second stably integrated nucleic acid molecule comprising the nucleic acid sequences encoding for Rep78 or Rep68; Rep52 or Rep40; VP1; VP2; VP3; and AAP.

94. The engineered cell of claim 93, wherein the second stably integrated nucleic acid molecule comprises the nucleic acid sequence encoding for Rep52, wherein the nucleic acid sequence encoding for Rep52 comprises an amino acid sequence of SEQ ID NO: 6.

95. The engineered cell of claim 93, wherein the second stably integrated nucleic acid molecule comprises the nucleic acid sequence encoding for Rep40, wherein the nucleic acid sequence encoding for Rep40 comprises an amino acid sequence of SEQ ID NO: 7.

96. The engineered cell of any one of claims 93-95, wherein the second stably integrated nucleic acid molecule comprises an amino acid sequence encoding for Rep78, wherein the nucleic acid sequence encoding for Rep78 comprises an amino acid sequence of SEQ ID NO: 8.

97. The engineered cell of any one of claims 93-95, wherein the second stably integrated nucleic acid molecule comprises an amino acid sequence encoding for Rep68, wherein the nucleic acid sequence encoding for Rep68 comprises an amino acid sequence of SEQ ID NO: 9.

98. The engineered cell of any one of claims 93-97, wherein the second stably integrated nucleic acid molecule comprises an amino acid sequence encoding for VP1, VP2, or VP3.

99. The engineered cell of claim 98, wherein the nucleic acid sequence encoding for VP1 comprises an amino acid sequence of SEQ ID NO: 14.

100. The engineered cell of claim 98 or claim 99, wherein the nucleic acid sequence encoding for VP2 comprises an amino acid sequence of SEQ ID NO: 15.

101. The engineered cell of any one of claims 98-100, wherein the nucleic acid sequence encoding for VP3 comprises an amino acid sequence of SEQ ID NO: 16.

102. The engineered cell of any one of claims 93-101, wherein the second stably integrated nucleic acid molecule comprises a nucleic acid sequence encoding for AAP.

103. The engineered cell of claim 102, wherein the nucleic acid sequence encoding for AAP comprises the amino acid sequence of SEQ ID NO: 17.

104. The engineered cell of any one of claims 93-103, wherein the one or more stably integrated nucleic acid molecules comprises a third stably integrated nucleic acid molecule comprising the nucleic acid sequences encoding for E2A, E4Orf6, and VARNA.

105. The engineered cell of claim 104, wherein the nucleic acid sequence encoding for E2A comprises the amino acid sequence of SEQ ID NO: 10.

106. The engineered cell of claim 104 or claim 105, wherein the nucleic acid sequence encoding for E4Orf6 comprises the amino acid sequence of SEQ ID NO: 11.

107. The engineered cell of any one of claims 104-106, wherein the nucleic acid sequence encoding for VARNA comprises the amino acid sequence of SEQ ID NO: 13.

108. The engineered cell of any one of claims 104-107, wherein the third stably integrated nucleic acid molecule further comprises a selection marker that is operably linked to a promoter.

109. The engineered cell of any one of claims 104-108, wherein the nucleic acid sequence encoding for the one or more shRNAs is operably linked to a chemically inducible promoter, and wherein the engineered cell further comprises a fourth stably integrated nucleic acid molecule, wherein the fourth stably integrated nucleic acid molecule comprises a nucleic acid sequence encoding for a transcriptional activator that, when expressed in the presence of a small molecule inducer, binds to the chemically inducible promoter.

110. The engineered cell of claim 109, wherein the transcriptional activator is selected from the group consisting of Tet-On 3G, TetOff-Advanced, VanR-VP16, TtgR-VP16, PhIF-VP16, and the cumate-responsive transactivators cTA and rcTA.

111. The engineered cell of claim 109 or claim 110, wherein the small molecule inducer is selected from the group consisting of lactose, arabinose, and doxycycline.

112. The engineered cell of any one of claims 109-111, wherein the transcriptional activator is TET-On 3G and the small molecule inducer is doxycycline.

113. The engineered cell of any one of claims 82-112, wherein the engineered cell is HEK293 cell or HeLa cell.

114. A kit comprising the engineered cell of any one of claims 82-113.

115. The kit of claim 114, further comprising a nucleic acid molecule comprising, from 5′ to 3′: (i) a nucleic acid sequence of a 5′ inverted tandem repeat; (ii) a multiple cloning site; and (iii) a nucleic acid sequence of a 3′ inverted tandem repeat.

116. The kit of claim 115, wherein the nucleic acid molecule is a plasmid or a vector.

117. A method for AAV production, comprising culturing the engineered cell of any one of claims 109-112 in the presence of the small molecule inducer corresponding to the transcriptional activator that binds to the chemically inducible promoter, thereby inducing AAV production.

118. The method of claim 117, wherein the small molecule inducer is selected from the group consisting of lactose, arabinose, and doxycycline.
